(19) United States
(12) Patent Application Publication
(10) Pub. No.: US 2022/0023306 A1
(43) Pub. Date: Jan. 27, 2022

(54) USE OF A PPAR-DELTA AGONIST IN THE TREATMENT OF FATTY ACID OXIDATION DISORDERS (FAOD)

(71) Applicant: Reneo Pharmaceuticals, Inc., San Diego, CA (US)

(72) Inventors: Colin O'CARROLL, San Diego, CA (US); Niall O'DONNELL, San Diego, CA (US); Lynn PURKINS, San Diego, CA (US); Alex DORENBAUM, San Diego, CA (US)

(21) Appl. No.: 17/428,094

(22) PCT Filed: Feb. 3, 2020
(86) PCT No.: PCT/US2020/016430

§ 371 Date: Aug. 3, 2021

Related U.S. Application Data
(60) Provisional application No. 62/800,995, filed on Feb. 4, 2019.

(51) Int. Cl. A61K 31/5375; A61K 31/415; A61K 31/192; A61P 3/00; A61K 31/09; A61K 31/455; A61K 31/525; A61K 31/197; A61K 31/4188; A61K 31/51; A61K 31/4415; A61K 31/385; A61K 31/19; A61K 31/225; A61K 33/30; A61K 31/122; A61K 31/355; A61K 31/375; A61K 31/714; A61K 31/519; G01N 33/497

(52) U.S. Cl. CPC A61K 31/5375; G01N 33/497; A61K 31/192; A61P 3/00; A61K 31/09; A61K 31/455; A61K 31/525; A61K 31/197; A61K 31/4188; A61K 31/51; A61K 31/4415; A61K 31/385; A61K 31/19; A61K 31/225; A61K 33/30; A61K 31/122; A61K 31/355; A61K 31/375; A61K 31/714; A61K 31/519; A61K 31/415

(57) ABSTRACT

Described herein is the use of PPARδ agonists in the treatment of fatty acid oxidation disorders.

### CROSS-REFERENCE

This application claims the benefit of U.S. Provisional Patent Application No. 62/800,995 filed on Feb. 4, 2019, which is incorporated herein by reference in its entirety.

### FIELD OF THE INVENTION

Described herein are methods of using a peroxisome proliferator-activated receptor delta (PPAR-delta) agonist in the treatment or prevention of fatty acid oxidation disorders (FAOD).

### BACKGROUND OF THE INVENTION

Healthy mitochondria are vital to normal cellular activities. Mitochondrial dysfunction drives the pathogenesis of a wide variety of medical disorders, including acute conditions and chronic diseases. Distinct aspects of mitochondrial function, for example, bioenergetics, dynamics, and cellular signaling are well described and impairments in these activities likely contribute to disease pathogenesis. Impairments of mitochondrial function result in a family of disorders termed fatty acid oxidation disorders. PPAR-delta, a member of the nuclear regulatory superfamily of ligand-activating transcriptional regulators, is expressed throughout the body. PPAR-delta agonists induce genes related to fatty acid oxidation and mitochondrial biogenesis. PPAR-delta also has anti-inflammatory properties.

### SUMMARY OF THE INVENTION

In one aspect, described herein are methods for treating a fatty acid oxidation disorders (FAOD) in a mammal comprising administering to the mammal with a fatty acid oxidation disorder (FAOD) a peroxisome proliferator-activated receptor delta (PPARδ) agonist compound.

In another aspect, described herein is a method for improving whole-body fatty acid oxidation in a mammal with a fatty acid oxidation disorder (FAOD) comprising administering to the mammal with a fatty acid oxidation disorder (FAOD) a peroxisome proliferator-activated receptor delta (PPARδ) agonist compound.

In another aspect, described herein is a method of modulating peroxisome proliferator-activated receptor delta (PPARδ) activity in a mammal with a fatty acid oxidation disorder (FAOD) comprising administering to the mammal with the fatty acid oxidation disorder (FAOD) a proliferator-activated receptor delta (PPARδ) agonist compound.

In some embodiments, modulating peroxisome proliferator-activated receptor delta (PPARδ) activity comprises activating peroxisome proliferator-activated receptor delta (PPARδ).

In some embodiments, modulating peroxisome proliferator-activated receptor delta (PPARδ) activity comprises increasing peroxisome proliferator-activated receptor delta (PPARδ) activity.

In yet another aspect, described herein is a method for increasing fatty acid oxidation (FAO) in a mammal with a fatty acid oxidation disorder (FAOD) comprising administering to the mammal with the fatty acid oxidation disorder (FAOD) a proliferator-activated receptor delta (PPARδ) agonist compound.

In some embodiments, the peroxisome proliferator-activated receptor delta (PPARδ) agonist compound is administered to the mammal in an amount sufficient for normalizing FAO capacities in the mammal, up-regulating gene expression of any one of the enzymes or proteins involved in FAO, or a combination thereof.

In some embodiments, normalizing FAO capacities in the mammal comprises increasing FAO capacities to sufficient levels for ameliorating or reducing the severity of any one of symptoms of any one of the fatty acid oxidation disorders described herein.

In one aspect, described herein is a method for treating a fatty acid oxidation disorder (FAOD) in a mammal comprising administering to the mammal with a FAOD a peroxisome proliferator-activated receptor delta (PPARδ) agonist compound.

In some embodiments, treating FAOD comprises improving whole-body fatty acid oxidation (FAO) in the mammal, improving the mammal's exercise tolerance, decreasing pain, decreasing fatigue, or a combination thereof.

In some embodiments, improving the mammal's exercise tolerance comprises increasing the distance walked in about a 12-minute walk test. In some embodiments, the distance walked in such a 12 minute walk test increases by at least about 1 meter, at least about 5 meters, at least about 10 meters, at least about 20 meters, at least about 30 meters, at least about 40 meters, at least about 50 meter, at least about 60 meters, at least about 70 meters, at least about 80 meters, at least about 90 meters, at least about 100 meters, or more than about 100 meters.

As used herein the term “about” means within +10% of the value.

In some embodiments, improving the mammal's exercise tolerance comprises decreases in heart rate during the about 12-minute walk test. In some embodiments, heart rate decreases by 1 heart beat per minute, by 2 heart beats per minute, by 3 heart beats per minute, by 4 heart beats per minute, by 5 heart beats per minute, by at least about 10 heart beats per minute, or by at least about 20 heart beats per minute.

In some embodiments, improving the mammal's exercise tolerance comprises decreases in measured respiratory exchange ratios (RER).

In some embodiments, improving whole-body fatty acid oxidation in the mammal comprises increasing fatty acid oxidation (FAO) in the mammal. In some embodiments, increasing fatty acid oxidation (FAO) in the mammal comprises increases in the amount of exhaled ¹³CO₂ from the mammal after consuming a meal comprising ¹³C-enriched fatty acids. In some embodiments, the amount of exhaled ¹³CO₂ increases by at least about 5%, about 10%, about 15%, about 20%, about 25%, about 30%, about 40%, about 50%, about 60%, about 70%, about 80%, about 90%, or more than about 90% as compared to a mammal who is fed a meal comprising ¹³C-enriched fatty acids and not administered a PPARδ agonist compound. In some embodiments, the amount of exhaled ¹³CO₂ increases by at least about 5%, about 10%, about 15%, about 20%, about 25%, about 30%, about 40%, about 50%, about 60%, about 70%, about 80%, about 90%, or more than about 90% as compared to a mammal who is not fed a meal comprising ¹³C-enriched fatty acids. In some embodiments, the amount of exhaled ¹³CO₂ increases by at least about 5%, about 10%, about 15%, about 20%, about 25%, about 30%, about 40%, about 50%, about 60%, about 70%, about 80%, about 90%, or more than about 90% as compared to a mammal who is not fed a meal comprising ¹³C-enriched fatty acids and not administered a PPARδ agonist compound.

In some embodiments, administration of the PPARδ agonist compound to the mammal normalizes FAO capacities in the mammal, up-regulates gene expression of any one of the enzymes or proteins involved in FAO, increases the activity of an enzyme or protein involved in FAO, or a combination thereof.

In some embodiments, the peroxisome proliferator-activated receptor delta (PPARδ) agonist compound is administered to the mammal in an amount sufficient for increasing the activity of mutated enzymes or proteins involved in FAO. In some embodiments, the peroxisome proliferator-activated receptor delta (PPARδ) agonist compound is administered to the mammal in an amount sufficient for increasing the activity of mutated but catalytically active enzymes or proteins involved in FAO.

In some embodiments, the fatty acid oxidation disorder comprises defects in the enzymes or proteins involved in the entry of long-chain fatty acids into mitochondria, intramitochondrial β-oxidation defects of long-chain fatty acids affecting membrane bound enzymes, β-oxidation defects of short- and medium-chain fatty acids affecting enzymes of the mitochondrial matrix, defects in the enzymes or proteins involved with electron transfer to the respiratory chain from mitochondrial β-oxidation, or a combination thereof.

In some embodiments, the fatty acid oxidation disorder (FAOD) comprises carnitine transporter deficiency, carnitine/acylcarnitine translocase deficiency, carnitine palmitoyl transferase deficiency Type 1, carnitine palmitoyl transferase deficiency Type 2, glutaric acidemia Type 2, long-chain 3-hydroxyacyl CoA dehydrogenase deficiency, medium-chain acyl CoA dehydrogenase deficiency, short-chain acyl CoA dehydrogenase deficiency, short-chain 3-hydroxyacyl CoA dehydrogenase deficiency, trifunctional protein deficiency, or very long-chain acyl CoA dehydrogenase deficiency, or a combination thereof.

In some embodiments, the fatty acid oxidation disorder comprises carnitine palmitoyltransferase II (CPT2) deficiency, very long-chain Acyl-CoA dehydrogenase (VLCAD) deficiency, long-chain 3-hydroxyacyl-CoA dehydrogenase (LCHAD) deficiency, Trifunctional Protein (TFP) Deficiency; or a combination thereof.

In another aspect, described herein is a method of increasing activity of an enzyme or protein of the mitochondrial fatty acid beta-oxidation pathway in a mammal comprising administering a PPARS agonist compound to a mammal with a mutation or deficiency in an enzyme or protein of the mitochondrial fatty acid beta-oxidation pathway.

In yet another aspect, described herein is a method of increasing activity of an enzyme or protein of the mitochondrial fatty acid beta-oxidation pathway in a mammal comprising administering a PPARS agonist compound to a mammal with a deficiency in the activity of an enzyme or protein of the mitochondrial fatty acid beta-oxidation pathway.

In some embodiments, the deficiency in the activity of the enzyme or protein of the mitochondrial fatty acid beta-oxidation pathway results from a mutation in any one of the enzyme or protein of the mitochondrial fatty acid beta-oxidation pathway.

In some embodiments, the enzyme or protein of the mitochondrial fatty acid beta-oxidation pathway is short-chain acyl-CoA dehydrogenase (SCAD), medium-chain acyl-CoA dehydrogenase (MCAD), long-chain 3-hydroxyacyl-CoA dehydrogenase (LCHAD), very long-chain acyl-CoA dehydrogenase (VLCAD), mitochondrial trifunctional protein (TFP), carnitine transporter (CT), Carnitine palmitoyltransferase I (CPT I), carnitine-acylcarnitine translocase (CACT), carnitine palmitoyltransferase II (CPT II), isolated long-chain L3-hydroxyl-CoA dehydrogenase, medium-chain L3-hydroxyl-CoA dehydrogenase, short-chain L3-hydroxyl-CoA dehydrogenase, medium-chain 3-ketoacylCoA thiolase, or long-chain 3-ketoacylCoA thiolase (LCKAT).

In some embodiments, the mutation is K304E of MCAD; L540P, V174M, E609K, or combination thereof, of VLCAD; E510Q of TFP-alpha subunit (HADHA); R247C of TFP-beta subunit (HADHB); or a combination thereof.

In some embodiments, the mutation is a nucleotide mutation in the gene encoding VLCAD. In some embodiments, the mutation is 842C>A, 848T>C, 865G>A, 869G>A, 881G>A, 897G>T, 898A>G, 950T>C, 956C>A, 1054A>G, 1096C>T, 1097G>A, 1117A>T, 1001 T>G, 1066A>G, 1076C>T, 1153C>T, 1213G>C, 1146G>C, 13110T>C, 1322G>A, 1358G>A 1360G>A, 1372T>C, 1258A>C, 1388G>A, 1405C>T, 1406G>A, 1430G>A, 1349G>A, 1505T>C, 1396G>T, 1613G>C, 1600G>A, 1367G>A, 1375C>T, 1376G>A, 1532G>A, 1619T>C, 1804C>A, 1844G>A, 1825G>A, 1844G>A, 1837C>G, or a combination thereof.

In some embodiments, the mammal has one or more symptoms typically associated with a fatty acid oxidation disorder. In some embodiments, symptoms typically associated with a fatty acid oxidation disorder include, but are not limited to: elevated creatine kinase (CPK) levels, hepatic dysfunction, cardiomyopathy, hypoglycemia, rhabdomyolysis, acidosis, decreased muscle tone (hypotonia), muscle weakness, exercise intolerance, or combinations thereof.

In some embodiments, the PPARδ agonist binds to and activates the cellular PPARδ and does not substantially activate the cellular peroxisome proliferator activated receptors-alpha (PPARα) and -gamma (PPARγ). In some embodiments, the PPARδ agonist compound is a phenoxyalkylcarboxylic acid compound. In some embodiments, the PPARδ agonist compound is a phenoxyethanoic acid compound, phenoxypropanoic acid compound, phenoxybutanoic acid compound, phenoxypentanoic acid compound, phenoxyhexanoic acid compound, phenoxyoctanoic acid compound, phenoxynonanoic acid compound, or phenoxydecanoic acid compound.

In some embodiments, the PPARδ agonist compound is a phenoxyethanoic acid compound or a phenoxyhexanoic acid compound. In some embodiments, the PPARδ agonist compound is an allyloxyphenoxyethanoic acid acid compound.

In some embodiments, the PPARδ agonist is a compound selected from the group consisting of (Z)-[2-Methyl-4-[3-(4-methylphenyl)-3-[4-[3-(morpholin-4-yl)propynyl]phenyl]allyloxy]-phenoxy]acetic acid; (E)-[2-Methyl-4-[3-[4-[3-(pyrazol-1-yl)prop-1-ynyl]phenyl]-3-(4-trifluoromethylphenyl)-allyloxy]phenoxy]acetic acid; (E)-[4-[3-(4-Fluorophenyl)-3-[4-[3-(morpholin-4-yl)propynyl]phenyl]allyloxy]-2-methyl-phenoxy]acetic acid; (E)-[2-Methyl-4-[3-[4-[3-(morpholin-4-yl)propynyl]phenyl]-3-(4-trifluoromethylphenyl)allyloxy]-phenoxy]acetic acid; (E)-[4-[3-(4-Chlorophenyl)-3-[4-[3-(morpholin-4-yl)propynyl]phenyl]allyloxy]-2-methyl-phenoxy]acetic acid; (E)-[4-[3-(4-Chlorophenyl)-3-[4-[3-(morpholin-4-yl)propynyl]phenyl]allyloxy]-2-methylphenyl]-propionic acid; {4-[3,3-Bis-(4-bromo-phenyl)-allyloxy]-2-methyl-phenoxy}-acetic acid; or a pharmaceutically acceptable salt thereof.

In some embodiments, the PPARδ agonist is a compound selected from the group consisting of (Z)-[2-Methyl-4-[3-(4-methylphenyl)-3-[4-[3-(morpholin-4-yl)propynyl]phenyl]allyloxy]-phenoxy]acetic acid; (E)-[2-Methyl-4-[3-[4-[3-(pyrazol-1-yl)prop-1-ynyl]phenyl]-3-(4-trifluoromethylphenyl)-allyloxy]phenoxy]acetic acid; (E)-[4-[3-(4-Fluorophenyl)-3-[4-[3-(morpholin-4-yl)propynyl]phenyl]allyloxy]-2-methyl-phenoxy]acetic acid; (E)-[2-Methyl-4-[3-[4-[3-(morpholin-4-yl)propynyl]phenyl]-3-(4-trifluoromethylphenyl)allyloxy]-phenoxy]acetic acid; (E)-[4-[3-(4-Chlorophenyl)-3-[4-[3-(morpholin-4-yl)propynyl]phenyl]allyloxy]-2-methyl-phenoxy]acetic acid; (E)-[4-[3-(4-Chlorophenyl)-3-[4-[3-(morpholin-4-yl)propynyl]phenyl]allyloxy]-2-methylphenyl]-propionic acid; {4-[3-Isobutoxy-5-(3-morpholin-4-yl-prop-1-ynyl)-benzylsulfanyl]-2-methyl-phenoxy}-acetic acid; {4-[3-Isobutoxy-5-(3-morpholin-4-yl-prop-1-ynyl)-phenylsulfanyl]-2-methyl-phenoxy}-acetic acid; {4-[3,3-Bis-(4-bromo-phenyl)-allyloxy]-2-methyl-phenoxy}-acetic acid; 2-[2-methyl-4-[[3-methyl-4-[[4-(trifluoromethyl)phenyl]methoxy]phenyl]thio]phenoxy]-acetic acid; (S)-4-[cis-2,6-dimethyl-4-(4-trifluoromethoxy-phenyl)piperazine-1-sulfonyl]-indan-2-carboxylic acid or a tosylate salt thereof (KD-3010); 4-butoxy-a-ethyl-3-[[[2-fluoro-4-(trifluoromethyl)benzoyl]amino]methyl]-benzenepropanoic acid (TIPP-204); 2-[2-methyl-4-[[[4-methyl-2-[4-(trifluoromethyl)phenyl]-5-thiazolyl]methyl]thio]phenoxy]-acetic acid (GW-501516); 2-[2,6 dimethyl-4-[3-[4-(methylthio)phenyl]-3-oxo-1(E)-propenyl]phenoxyl]-2-methylpropanoic acid (GFT-505); {2-methyl-4-[5-methyl-2-(4-trifluoromethyl-phenyl)-2H-[1,2,3]triazol-4-ylmethylsylfanyl]-phenoxy}-acetic acid; (R)-3-methyl-6-(2-((5-methyl-2-(4-(trifluoromethyl)phenyl)-1H-imidazol-1-yl)methyl)phenoxy)hexanoic acid; (R)-3-methyl-6-(2-((5-methyl-2-(6-(trifluoromethyl)pyridin-3-yl)-1H-imidazol-1-yl)methyl)phenoxy)hexanoic acid; 2-(2-methyl-4-(((2-(4-(trifluoromethyl)phenyl)-2H-1,2,3-triazol-4-yl)methyl)thio)phenoxy)acetic acid; and (R)-2-(4-((2-ethoxy-3-(4-(trifluoromethyl)phenoxy)propyl)thio)phenoxy)acetic acid; or a pharmaceutically acceptable salt thereof.

In some embodiments, the PPARδ agonist is a compound selected from the group consisting of PPARδ agonist is a compound selected from the group consisting of: (Z)-[2-Methyl-4-[3-(4-methylphenyl)-3-[4-[3-(morpholin-4-yl)propynyl]phenyl]allyloxy]-phenoxy]acetic acid; (E)-[2-Methyl-4-[3-[4-[3-(pyrazol-1-yl)prop-1-ynyl]phenyl]-3-(4-trifluoromethylphenyl)-allyloxy]phenoxy]acetic acid; (E)-[4-[3-(4-Fluorophenyl)-3-[4-[3-(morpholin-4-yl)propynyl]phenyl]allyloxy]-2-methyl-phenoxy]acetic acid; (E)-[2-Methyl-4-[3-[4-[3-(morpholin-4-yl)propynyl]phenyl]-3-(4-trifluoromethylphenyl)allyloxy]-phenoxy]acetic acid; (E)-[4-[3-(4-Chlorophenyl)-3-[4-[3-(morpholin-4-yl)propynyl]phenyl]allyloxy]-2-methyl-phenoxy]acetic acid; (E)-[4-[3-(4-Chlorophenyl)-3-[4-[3-(morpholin-4-yl)propynyl]phenyl]allyloxy]-2-methylphenyl]-propionic acid; {4-[3-Isobutoxy-5-(3-morpholin-4-yl-prop-1-ynyl)-benzylsulfanyl]-2-methyl-phenoxy}-acetic acid; {4-[3-Isobutoxy-5-(3-morpholin-4-yl-prop-1-ynyl)-phenylsulfanyl]-2-methyl-phenoxy}-acetic acid; and {4-[3,3-Bis-(4-bromo-phenyl)-allyloxy]-2-methyl-phenoxy}-acetic acid; or a pharmaceutically acceptable salt thereof.

In some embodiments, the PPARδ agonist is (E)-[4-[3-(4-Fluorophenyl)-3-[4-[3-(morpholin-4-yl)propynyl]phenyl]allyloxy]-2-methyl-phenoxy]acetic acid (Compound I) or a pharmaceutically acceptable salt thereof.

In some embodiments, (E)-[4-[3-(4-Fluorophenyl)-3-[4-[3-(morpholin-4-yl)propynyl]phenyl]allyloxy]-2-methyl-phenoxy]acetic acid or a pharmaceutically acceptable salt thereof, is administered to the mammal at a dose of about 10 mg to about 500 mg, about 50 mg to about 200 mg, or about 75 mg to about 125 mg.

In some embodiments, the PPARδ agonist is (E)-[4-[3-(4-Fluorophenyl)-3-[4-[3-(morpholin-4-yl)propynyl]phenyl]allyloxy]-2-methyl-phenoxy]acetic acid or a pharmaceutically acceptable salt thereof, and is administered to the mammal at a dose of about 10 mg to about 500 mg. In some embodiments, the PPARδ agonist is (E)-[4-[3-(4-Fluorophenyl)-3-[4-[3-(morpholin-4-yl)propynyl]phenyl]allyloxy]-2-methyl-phenoxy]acetic acid or a pharmaceutically acceptable salt thereof, and is administered to the mammal at a dose of about 50 mg to about 200 mg. In some embodiments, the PPARδ agonist is (E)-[4-[3-(4-Fluorophenyl)-3-[4-[3-(morpholin-4-yl)propynyl]phenyl]allyloxy]-2-methyl-phenoxy]acetic acid (Compound I) or a pharmaceutically acceptable salt thereof, and is administered to the mammal at a dose of about 75 mg to about 125 mg.

In some embodiments, a PPARδ agonist (e.g. Compound 1, or a pharmaceutically acceptable salt thereof), is systemically administered to the mammal with a fatty acid oxidation disorder (FAOD). In some embodiments, the PPARδ agonist is administered to the mammal orally, by injection or intravenously. In some embodiments, the PPARδ agonist is administered to the mammal in the form of an oral solution, oral suspension, powder, pill, tablet or capsule.

In one aspect, described herein is a pharmaceutical composition comprising PPARδ agonist and at least one pharmaceutically acceptable excipient. In some embodiments, the pharmaceutical composition is formulated for administration to a mammal by intravenous administration, subcutaneous administration, oral administration, inhalation, nasal administration, dermal administration, or ophthalmic administration. In some embodiments, the pharmaceutical composition is formulated for administration to a mammal by intravenous administration, subcutaneous administration, or oral administration. In some embodiments, the pharmaceutical composition is formulated for administration to a mammal by oral administration. In some embodiments, the pharmaceutical composition is in the form of a tablet, a pill, a capsule, a liquid, a suspension, a gel, a dispersion, a solution, an emulsion, an ointment, or a lotion. In some embodiments, the pharmaceutical composition is in the form of a tablet, a pill, or a capsule.

In one aspect, described herein is a method of treatment or prevention of any one of the fatty acid oxidation disorders (FAOD) described herein comprising administering a therapeutically effective amount of a PPARδ agonist to a mammal in need thereof.

In any of the aforementioned aspects are further embodiments in which the effective amount of the PPARδ agonist (e.g. Compound 1, or a pharmaceutically acceptable salt thereof), is: (a) systemically administered to the mammal; and/or (b) administered orally to the mammal; and/or (c) intravenously administered to the mammal; and/or (d) administered by injection to the mammal; and/or (e) administered non-systemically or locally to the mammal.

In any of the aforementioned aspects are further embodiments comprising single administrations of the effective amount of the PPARδ agonist (e.g. Compound 1, or a pharmaceutically acceptable salt thereof), including further embodiments in which the PPARδ agonist (e.g. Compound 1, or a pharmaceutically acceptable salt thereof), is administered once daily to the mammal or is administered to the mammal multiple times over the span of one day. In some embodiments, the PPARδ agonist (e.g. Compound 1, or a pharmaceutically acceptable salt thereof), is administered on a continuous dosing schedule. In some embodiments, the PPARδ agonist is administered on a continuous daily dosing schedule.

In any of the aforementioned aspects involving the treatment of a disease or condition are further embodiments comprising administering at least one additional agent in addition to the administration of a PPARδ agonist (e.g. Compound 1, or a pharmaceutically acceptable salt thereof). In various embodiments, each agent is administered in any order, including simultaneously.

In some embodiments, the at least one additional therapeutic is ubiquinol, ubiquinone, niacin, riboflavin, creatine, L-carnitine, acetyl-L-carnitine, biotin, thiamine, pantothenic acid, pyridoxine, alpha-lipoic acid, n-heptanoic acid, CoQ10, vitamin E, vitamin C, methylcobalamin, folinic acid, N-acetyl-L-cysteine (NAC), zinc, folinic acid/leucovorin calcium, resveratrol, or a combination thereof. In some embodiments, the at least one additional therapeutic is an odd-chain fatty acid, odd-chain fatty ketone, L-carnitine, or combinations thereof. In some embodiments, the at least one additional therapeutic is triheptanoin, n-heptanoic acid, a triglyceride, or a salt or thereof, or combinations thereof.

In any of the embodiments disclosed herein, the mammal is a human.

In some embodiments, the PPARδ agonist (e.g. Compound 1, or a pharmaceutically acceptable salt thereof), is administered to a human. In some embodiments, the PPARδ agonist (e.g. Compound 1, or a pharmaceutically acceptable salt thereof), is orally administered.

Articles of manufacture, which include packaging material, a compound described herein, or a pharmaceutically acceptable salt thereof, within the packaging material, and a label that indicates that a PPARδ agonist (e.g. Compound 1, or a pharmaceutically acceptable salt thereof), is used for modulating the activity of PPARS, or for the treatment, prevention or amelioration of one or more symptoms of a fatty acid oxidation disorder (FAOD) that would benefit from modulation of PPARδ activity, are provided.

Other objects, features and advantages of the compounds, methods and compositions described herein will become apparent from the following detailed description. It should be understood, however, that the detailed description and the specific examples, while indicating specific embodiments, are given by way of illustration only, since various changes and modifications within the spirit and scope of the instant disclosure will become apparent to those skilled in the art from this detailed description.

### BRIEF DESCRIPTION OF THE FIGURES

FIG. 1 shows the effect of Compound 1 (50 mg once a day for 12 weeks) on the 12-minute walk test in patients with genetically diagnosed long chain FAOD with symptoms of myopathy.

### DETAILED DESCRIPTION

Mitochondria are the main site for the oxidation of fatty acids and triglycerides through a series of four enzyme reactions called β-oxidation. The β-oxidation pathway is a cyclic process in which two carboxy-terminal carbon atoms are released from fatty acids as acetyl-CoA units each time a cycle is fully completed. The acetyl-CoA can enter the citric acid cycle and the electron carriers deliver the electrons to the electron transport chain. Fatty acid oxidation (FAO) both produces acetyl-CoA to fuel the tricarboxylic acid (TCA) cycle and ketogenesis, and reduces flavin adenine dinucleotide (to FADH2) and nicotinamide adenine dinucleotide (to NADH); these reduced products directly feed into the respiratory chain. As the acyl-CoA gets shorter, its physicochemical properties change. To be able to fully degrade fatty acids, the 3-oxidation machinery harbors different chain length-specific enzymes. Inherited defects for most of the β-oxidation enzymes have been identified and characterized (see for example, S. M. Houten, et al., The Biochemistry and Physiology of Mitochondrial Fatty Acid β-Oxidation and Its Genetic Disorders. Annual Review of Physiology 2016 78:1, 23-44).

FAO is crucial for ATP production in muscle, particularly during exercise. The sources of fatty acids differ depending on the exercise intensity, with the contribution of free fatty acids increasing with exercise intensity. Mutations in any of the enzymes involved in FAO, in some cases, lead to a variety of clinical symptoms in particular during fasting and in organs with high energy needs. During infancy, patients, in some cases, present with cardiac symptoms such as dilated or hypertrophic cardiomyopathy and/or arrhythmias. Alternatively, FAO defects, in some cases, present as a milder, later (‘adult’) onset disease, characterized by exercise-induced myopathy and rhabdomyolysis. Human inherited defects have been described for almost all enzymes and transporters involved in FAO.

In most FAO defects, disease-causing mutations have been characterized that result in absent or non-functional protein, or variable levels of residual enzyme activity. The PPARs (PPAR-α, PPAR-δ, PPAR-γ) are known for their transcriptional regulation of FAO. Activation of PPARs, in some cases, trigger an up-regulation of gene expression of the enzymes involved in FAO resulting in an increase in residual enzyme activity and thereby correction of FAO flux in treated cells. This is the case for the defect in CPT2. CPT2 is an inner mitochondrial membrane enzyme involved in the transfer of long-chain fatty acids from cytosol to the mitochondrial matrix, in concert with its outer membrane counterpart, CPT1. Using the PPAR agonist bezafibrate, pharmacological enhancement of a deficient enzyme could be achieved in cultured patient fibroblasts carrying mild mutations of the CPT2 gene (Djouadi, F., et al. Pediatr. Res. 54, 446-451, 2003). Bezafibrate is a pan-PPAR agonist with limited selectivity for any of the three receptor subtypes. In a follow up study (Djouadi, F., et al. J. Clin. Endocrinol. Metab. 90, 1791-1797, 2005) using cultured patient muscle cells, specific agonists of PPARδ (GW 072) and, to a lower extent, PPARα (GW 7647) stimulated FAO in control myoblasts. However, when tested in CPT2-deficient myoblasts, both bezafibrate and the PPARδ agonist were able to restore FAO, whereas the PPARα agonist had no effect. The PPARδ selective agonist increased residual CPT2 activity and normalized long-chain acylcarnitine production by deficient cells. In some embodiments, selective PPARδ agonists are therapeutic options for correction of FAO defects.

Pharmacological rescue of residual enzyme activity, in some cases, is potentially extended to other FAO gene defects, such as VLCAD, because the PPAR signaling pathway controls several enzymes of the β-oxidation pathway. For example, using the PPARδ agonist compound MA-0211, improvements in fatty acid oxidation were observed in fibroblasts derived from patients with very long-chain Acyl-CoA dehydrogenase (VLCAD), long-chain 3-hydroxylacyl-CoA dehydrogenase (LCHAD) and mitochondrial trifunctional protein (TFP) deficiencies were observed (see Goddeeris, M., et al., A Novel Small-Molecule PPARδ Modulator for the Treatment of Fatty Acid Oxidation Disorders. Poster Session presented at INFORM: International Network for Fatty Acid Oxidation Research and Management; Rio de Janeiro, Brazil, Sep. 4, 2017).

Using the VLCAD deficient cell line FB833, the following PPARδ agonist compounds were shown to increase VLCAD enzyme activity: 2-[2-Methyl-4-[[[4-methyl-2-[4-(trifluoromethyl)phenyl]-5-thiazolyl]methyl]thio]phenoxy]acetic acid (GW501516), [4-[[[2-[3-Fluoro-4-(trifluoromethyl)phenyl]-4-methyl-5-thiazolyl]methyl]thio]-2-methylphenoxy]acetic acid (GW0742 also known as GW610742), and [4-[3-(4-Acetyl-3-hydroxy-2-propylphenoxy)propoxy]phenoxy]acetic acid (L-165,0411) (See FIGS. 20 and 21 of International publication no. WO18093839).

In vitro studies with Compound 1 have demonstrated its ability to elicit a dose-dependent increase in fatty acid oxidation in human and rat muscle cell lines. In addition, Compound 1 treatment altered the expression patterns of several well-known PPARδ regulated genes in pathways important for fatty acid metabolism (CPT1b) and mitochondrial biogenesis (PGC1α) in vivo.

In vitro studies with cultured fibroblasts obtained from symptomatic patients with FAOD due to very long-chain acyl-CoA dehydrogenase (VLCAD) deficiency, Compound 1 increased VLCAD enzymatic activity. In some embodiments, Compound 1 increases the activity of mutated but catalytically active enzymes and transporters in the FAO pathway in subjects with a FAOD. In some embodiments, Compound 1 increases the activity of mutated but catalytically active enzymes and transporters in the FAO pathway in symptomatic patients with FAOD due to very long-chain acyl-CoA dehydrogenase (VLCAD) deficiency. In some embodiments, Compound 1 improves whole-body fatty acid oxidation, and thus decreases disease severity in VLCAD patients.

Described herein, in some embodiments, are methods of pharmacological rescue of residual enzyme activity of enzymes involved in the fatty acid β-oxidation pathway. In some embodiments, certain cells bearing mutations are expected to have some residual enzymatic activity. For example, in some embodiments, low residual enzymatic activity of VLCAD is observed in fibroblasts obtained from patients bearing missense mutations (Goetzman E S. Advances in the Understanding and Treatment of Mitochondrial Fatty Acid Oxidation Disorders. Curr Genet Med Rep. 2017; 5(3):132-142). In some embodiment, described herein are methods of increasing residual enzyme activity of one or more enzymes involved in the fatty acid β-oxidation pathway in a mammal with a FAOD comprising administering a PPARδ agonist (e.g. Compound 1, or a pharmaceutically acceptable salt thereof) to a mammal with a FAOD. In some embodiment, described herein are methods of increasing residual enzyme activity of one or more enzymes involved in the fatty acid β-oxidation pathway in a mammal with a FAOD by about 5%, about 10%, about 15%, about 20%, about 25%, about 30%, about 35%, about 40%, about 45%, about 50%, about 55%, about 60%, about 75%, about 80%, about 95%, about 100%, or more than 100% of the enzyme activity levels observed for a mammal without a FAOD comprising administering a PPARδ agonist (e.g. Compound 1, or a pharmaceutically acceptable salt thereof) to a mammal with a FAOD.

In some embodiments, deficiencies in FAO capacities are measured by comparing FAO capacities of a mammal identified as having a FAOD to the FAO capacities of a mammal without a FAOD (i.e. a control). In some embodiments, described herein are methods of increasing FAO capacities in a mammal with a FAOD comprising administering a PPARδ agonist (e.g. Compound 1, or a pharmaceutically acceptable salt thereof) to a mammal with a FAOD. In some embodiments, described herein are methods of increasing FAO capacities in a mammal with a FAOD by about 5%, about 10%, about 15%, about 20%, about 25%, about 30%, about 35%, about 40%, about 45%, about 50%, about 55%, about 60%, about 75%, about 80%, about 95%, about 100%, or more than 100% of the levels observed for a mammal without a FAOD. In some embodiments, described herein are methods of increasing FAO capacities in a mammal with a FAOD to a level substantially similar to that observed for a mammal without a FAOD comprising administering a PPARδ agonist (e.g. Compound 1, or a pharmaceutically acceptable salt thereof) to a mammal with a FAOD. In some embodiments, described herein are methods of restoring (i.e. normalizing) FAO capacities in a mammal with a FAOD to a level substantially similar to that observed for a mammal without a FAOD comprising administering a PPARδ agonist (e.g. Compound 1, or a pharmaceutically acceptable salt thereof) to a mammal with a FAOD.

In some embodiments, administration of a PPARδ agonist (e.g. Compound 1, or a pharmaceutically acceptable salt thereof), to a mammal with a FAOD restores (i.e. normalizes) a deficiency in the activity of one or more enzymes of proteins involved in the fatty acid β-oxidation pathway. In some embodiments, restoring activity comprises increasing the activity to substantially similar levels observed in a mammal without a FAOD.

Described herein, in some embodiments, are methods and compositions for treating a fatty acid oxidation (FAO) disorder. In some embodiments, the FAO disorder is caused by a mutation in a gene involved in FAO. In some embodiments, the mutation causes the gene to encode a non-functional protein or a protein with reduced activity. In some embodiments, methods comprise administering a peroxisome proliferator-activated receptor delta (PPARS). In some embodiments, administration of the PPARδ increases the expression of the gene involved in FAO. In some embodiments, administration of the PPARδ increases the activity of the protein involved in FAO.

Methods described herein, in some embodiments, comprise treating a FAO disorder caused by a mutation in a gene of interest. In some embodiments, the mutation is a gene mutation. In some embodiments, the mutation is a missense mutation, a nonsense mutation, an insertion, a deletion, a duplication, a frameshift mutation, a repeat expansion, a splicing mutation, or a whole gene deletion. In some embodiments, the FAO disorder is caused by one or more mutations in the gene of interest.

In some embodiments, the gene of interest is a gene involved in fatty acid oxidation. In some embodiments, the gene of interest encodes for a protein involved in fatty acid oxidation. In some embodiments, the gene of interest encodes for a protein that functions as a carnitine shuttle. In some embodiments, the gene of interest encodes for a protein that functions in the fatty acid oxidation cycle. In some embodiments, the gene of interest encodes for a protein that functions as an auxiliary enzyme. In some embodiments, the mutation in a gene of interest encodes for a protein with increased activity. In some embodiments, the mutation in a gene of interest encodes for a protein with reduced activity.

Methods described herein, in some embodiments, comprise treating a FAO disorder caused by a mutation in a gene of interest, wherein the gene of interest encodes for a protein that functions as a carnitine shuttle. Exemplary genes that encode for a protein that functions as a carnitine shuttle include, but not limited to, CPT1A, CPT1B, SLC25A20, CPT2, and SLC22A5. In some embodiments, the mutation is in CPT1A. In some embodiments, the mutation is in CPT1B.

In some embodiments, the mutation is in SLC25A20. In some embodiments, the mutation is in CPT2. In some embodiments, the mutation is in SLC22A5. In some embodiments, the mutation is in one or more genes selected from the group consisting of CPT1A, CPT1B, SLC25A20, CPT2, and SLC22A5.

CPT1A, also known as carnitine palmitoyltransferase 1A, encodes the CPT1A protein. CPT1B, also known as carnitine palmitoyltransferase 1, encodes the CPT1B protein. CTP1 is an outer-mitochondrial-membrane protein and catalyzes the transesterification of the acyl-CoA to acylcarnitine. In some embodiments, a mutation is in CPT1A. In some embodiments, a mutation in CPT1A results in a decrease or loss of activity in CPT1A. In some embodiments, a mutation is in CPT1A comprising a sequence as set forth in NCBI Reference Number NM_001031847.2. In some embodiments, a mutation in CPT1A is a mutation in a peptide sequence. In some embodiments, the mutation results in a missense substitution, a nonsense substitution (*), a coding silent substitution, deletion (del), an insertion (ins), or a frameshift (fs). In some embodiments, a mutation in CPT1A translates to amino acid positions in CPT1A selected from: R123, C304, T314, R316, F343, R357, E360, A414, D454, G465, P479, L484, Y498, G709, and G710, wherein the amino acids correspond to positions 123, 304, 314, 316, 343, 357, 360, 414, 454, 465, 479, 484, 498, 709 and 710 of SEQ ID NO: 6. In some embodiments, a mutation in CPT1A translates to one or more different amino acid positions of SEQ ID NO: 6. In some embodiments, the mutation in CPT1A, which translates to amino acid positions in CPT1A includes, but are not limited to, R123C, C304W, T314I, R316G, F343V, R357W, E360G, 395del, A414V, D454G, G465W, P479L, L484P, Y498C, G709E, and G710E.

In some embodiments, a mutation is in CPT1B. In some embodiments, a mutation is in CPT1B comprising a sequence as set forth in NCBI Reference Number NM_004377.3. In some embodiments, a mutation in CPT1B is a mutation in a peptide sequence. In some embodiments, the mutation results in a missense substitution, a nonsense substitution (*), a coding silent substitution, deletion (del), an insertion (ins), or a frameshift (fs). In some embodiments, a mutation in CPT1B translates to amino acid positions in CPT1B selected from: 166, G320, S427, E531, and S664, wherein the amino acids correspond to positions 66, 320, 427, 531, and 664 of SEQ ID NO: 7. In some embodiments, a mutation in CPT1B translates to one or more different amino acid positions of SEQ ID NO: 7. In some embodiments, the mutation in CPT1B, which translates to amino acid positions in CPT1B includes, but are not limited to, I66V, G320D, S427C, E531K, and S664Y.

SLC25A20, also known as solute Carrier Family 25 Member 20 or carnitine acylcarnitine translocase (CACT), encodes the CACT protein. CACT carries out the transport of acylcarnitines across the inner mitochondrial membrane in exchange for a free carnitine molecule. In some embodiments, a mutation is in SLC25A20 comprising a sequence as set forth in NCBI Reference Number NM_000387.6. In some embodiments, a mutation in SLC25A20 is a mutation in a peptide sequence. In some embodiments, the mutation results in a missense substitution, a nonsense substitution (*), a coding silent substitution, deletion (del), an insertion (ins), or a frameshift (fs). In some embodiments, a mutation in SLC25A20 translates to amino acid positions in CACT selected from: R133, D231, and Q238, wherein the amino acids correspond to positions 133, 231, and 238 of SEQ ID NO: 8. In some embodiments, a mutation in SLC25A20 translates to one or more different amino acid positions of SEQ ID NO: 8. In some embodiments, the mutation in SLC25A20, which translates to amino acid positions in CACT includes, but are not limited to, R133W, D231H, and Q238R.

CPT2, also known as carnitine O-palmitoyltransferase 2, encodes the CPT2 protein. CPT2 is a peripheral inner-mitochondrial-membrane protein and completes the fatty acid oxidation cycle by reconverting the acylcarnitine into an acyl-Co. In some embodiments, a mutation is in CPT2. In some embodiments, a mutation is in CPT2 comprising a sequence as set forth in NCBI Reference Number NM_000098.3. In some embodiments, a mutation in CPT2 is a mutation in a peptide sequence. In some embodiments, the mutation results in a missense substitution, a nonsense substitution (*), a coding silent substitution, deletion (del), an insertion (ins), or a frameshift (fs). In some embodiments, a mutation in CPT2 translates to amino acid positions in CPT2 selected from: P50, S113, R151, Y210, D213, M214, P227, R296, F383, F448, Y479, R503, G549, Q550, D553, G600, P604, Y628, and R631, wherein the amino acids correspond to positions 50, 113, 151, 210, 213, 214, 227, 296, 383, 448, 479, 503, 549, 550, 553, 600, 604, 628, and 631 of SEQ ID NO: 9. In some embodiments, a mutation in CPT2 translates to one or more different amino acid positions of SEQ ID NO: 9. In some embodiments, the mutation in CPT2, which translates to amino acid positions in CPT2 includes, but are not limited to, P50H, S113L, R151Q, Y210D, D213G, M214T, P227L, R296Q, F383Y, F448L, Y479F, R503C, G549D, Q550R, D553N, G600R, P604S, Y628S, and R631C.

SLC22A5, also known as solute carrier family 22 member 5, encodes OCTN2 protein. OCTN2 functions to transport carnitine across the plasma membrane. In some embodiments, a mutation is in SLC22A5. In some embodiments, a mutation is in SLC22A5 comprising a sequence as set forth in NCBI Reference Number NM_001308122.1. In some embodiments, a mutation in SLC22A5 is a mutation in a peptide sequence. In some embodiments, the mutation results in a missense substitution, a nonsense substitution (*), a coding silent substitution, deletion (del), an insertion (ins), or a frameshift (fs). In some embodiments, a mutation in SLC22A5 translates to amino acid positions in OCTN2 selected from: G12, G15, P16, F17, R19, L20, S26, S28, N32, A44, P46, C50, T66, R75, R83, S93, L95, G96, D115, D122, V123, E131, A142, P143, V151, R169, V175, M177, M179, L186, M205, N210, Y211, A214, T219, S225, R227, F230, S231, T232, G234, A240, G242, P247, R254, R257, T264, L269, S280, R282, W283, A301, I312, E317, I348, W351, S355, Y358, S362, L363, P398, R399, S412, V439, T440, A442, F443, V446, Y447, V448, Y449, E452, P455, G462, S467, T468, S470, R471, L476, P478, R488, and L507S, wherein the amino acid corresponds to 12, 15, 16, 17, 19, 20, 26, 28, 32, 44, 46, 50, 66, 75, 83, 93, 95, 96, 115, 122, 123, 131, 142, 143, 151, 169, 175, 177, 179, 186, 205, 210, 211, 214, 219, 225, 227, 230, 231, 232, 234, 240, 242, 247, 254, 257, 264, 269, 280, 282, 283, 301, 312, 317, 348, 351, 355, 358, 362, 363, 398, 399, 412, 439, 440, 442, 443, 446, 447, 448, 449, 452, 455, 462, 467, 468, 470, 471, 476, 478, 488, and 5070f SEQ ID NO: 10. In some embodiments, a mutation in SLC22A5 translates to one or more different amino acid positions of SEQ ID NO: 10. In some embodiments, the mutation in SLC22A5, which translates to amino acid positions in OCTN2 includes, but are not limited to 4-557del, G12S, G15W, P16L, F17L, R19P, L20H, 22del, S26N, S28I, N32S, A44V, P46L, P46S, C50Y, T66P, R75P, R83L, S93W, L95V, G96A, D115G, 117-557del, D122Y, V123G, E131D, 132-557del, 140-557del, A142S, P143L, V151M, R169P, R169Q, R169W, V175M, M177V, M179L, L186P, M205R, N210S, Y211C, A214V, T219K, S225L, R227H, F230L, S231F, T232M, G234R, A240T, G242V, P247R, 254-557del, R254Q, 256-557del, R257W, T264M, T264R, L269P, 275-557del, S280F, 282-557del, R282Q, W283C, W283R, 289-557del, 295-557del, A301D, 1312V, E317K, 319-557del, 1348T, W351R, S355L, Y358N, S362L, L363P, 387-557del, 394del, P398L, R399Q, R399W, S412G, V439G, T440M, A4421, F443V, V446F, Y447C, V448L, Y449D, E452K, P455R, G462V, S467C, T468R, S470F, R471C, R471H, R471P, L476R, P478L, R488C, R488H, and L507S.

Methods described herein, in some embodiments, comprise treating a FAO disorder caused by a mutation in a gene of interest, wherein the gene of interest encodes for a protein that functions in the fatty acid oxidation cycle. Exemplary genes that encode for a protein that functions in the fatty acid oxidation cycle include, but not limited to, ACADVL, ACADM, ACADS, HADHA, HADHB, ECHS1, HADH, ACAA2, ACAT1, ACADL, and ACAD9. In some embodiments, the mutation is in ACADVL. In some embodiments, the mutation is in ACADM. In some embodiments, the mutation is in ACADS. In some embodiments, the mutation is in HADHA. In some embodiments, the mutation is in HADHB. In some embodiments, the mutation is in ECHS1. In some embodiments, the mutation is in HADH. In some embodiments, the mutation is in ACAA2. In some embodiments, the mutation is in ACAT1. In some embodiments, the mutation is in ACADL. In some embodiments, the mutation is in ACAD9. In some embodiments, the mutation is in one or more genes selected from the group consisting of ACADVL, ACADM, ACADS, HADHA, HADHB, ECHS1, HADH, ACAA2, ACAT1, ACADL, and ACAD9.

ACADVL, also known as very long chain acyl-CoA dehydrogenase, encodes the VLCAD protein. VLCAD is a member of the aceyl-CoA dehydrogenase family and metabolizes aceyl-CoA's from long chain acyl CoA. In some embodiments, a mutation is in ACADVL. In some embodiments, a mutation is in ACADVL comprising a sequence as set forth in SEQ ID NO: 11. Exemplary mutations in the nucleotide sequence include, but are not limited to, 128G>A, 194C>T, 215C>T, 439C>T, 473C>A, 476A>G, 455G>A, 481G>A, 482C>T, 520G>A, 553G>A, 622G>A, 637G>C, 520G>A, 652G>A, 535G>T, 728T>G, A739G, 740A>C, c.637G>A, 753-2A>C, 7790>T, 664G>C, 689C>T, 739A>C transversion, 842C>A, 848T>C, 865G>A, 869G>A, 881G>A, 897G>T, 898A>G, 950T>C, 956C>A, 1054A>G, 1096C>T, 1097G>A, 1117A>T, 1001T>G, 1066A>G, 1076C>T, 1153C>T, 1213G>C, 1146G>C, 1310T>C, 1322G>A, 1358G>A, 1360G>A, 1372T>C, 1258A>C, 1388G>A, 1405C>T, 1406G>A, 1430G>A, 1349G>A, 1505T>C, 1396G>T, 1613G>C, 1600G>A, 1367G>A, 1375C>T, 1376G>A, 1532G>A, 1619T>C, 1804C>A, 1844G>A, 1825G>A, 1844G>A, and 1837C>G. In some embodiments, the mutation in the nucleotide sequence is 842C>A, 848T>C, 865G>A, 869G>A, 881G>A, 897G>T, 898A>G, 950T>C, 956C>A, 1054A>G, 1096C>T, 1097G>A, 1117A>T, 1001 T>G, 1066A>G, 1076C>T, 1153C>T, 1213G>C, 1146G>C, 1310T>C, 1322G>A, 1358G>A, 1360G>A, 1372T>C, 1258A>C, 1388G>A, 1405C>T, 1406G>A, 1430G>A, 1349G>A, 1505T>C, 1396G>T, 1613G>C, 1600G>A, 1367G>A, 1375C>T, 1376G>A, 1532G>A, 1619T>C, 1804C>A, 1844G>A, 1825G>A, 1844G>A, 1837C>G, or a combination thereof. In some embodiments, a mutation in ACADVL is a mutation in a peptide sequence. In some embodiments, the mutation results in a missense substitution, a nonsense substitution (*), a coding silent substitution, deletion (del), an insertion (ins), or a frameshift (fs). In some embodiments, a mutation in ACADVL translates to amino acid positions in VLCAD selected from: P65, S72, P147, T118, Q119, A161, V134, G145, G208, A213, E218, L243, K247, T260, G222, T230, V283, G289, M300, R366, 1373, M334, 1356, A359, R385, K382, M437, G439, G441, 1420, R450, D466, R459, R511, L540, E609, R615, and R613, wherein the amino acids correspond to positions 65, 72, 147, 118, 119, 161, 134, 145, 208, 213, 218, 243, 247, 260, 222, 230, 283, 289, 300, 366, 373, 334, 356, 359, 385, 382, 437, 439, 441, 420, 450, 466, 459, 511, 540, 609, 615, and 613 of SEQ ID NO: 22. In some embodiments, a mutation in ACADVL translates to one or more different amino acid positions of SEQ ID NO: 22.

In some embodiments, the mutation in ACADVL, which translates to amino acid positions in VLCAD includes, but are not limited to, G3D, P65L, S72F, P147S, T118N, Q119R, G152D, A121T, A161V, V134M, G145S, G168R, A173P, V174M, E178K, G179W, L203R, K207E, K207T, A213T, T220M, G222R, T2301, K247Q, A281D, G289R, G250D, G254E, K259N, M300V, V277A, M312V, R326C, R326H, I333F, M334R, I356V, A359V, R345W, D365H, K382N, M437T, G401D, R413Q, D414N, F418L, G423E, R429W, R429Q, C437Y, R450H, L462P, D466Y, R538P, E454K, R456H, R459W, R459Q, R511Q, L5621, R575Q, R615Q, and R613G. In some embodiments, the mutation in ACADVL, which translates to amino acid positions in VLCAD, is L540P, V174M, E609K, or combination thereof.

ACADM, also known as medium-chain specific acyl-CoA dehydrogenase, encodes the MCAD protein. MCAD is a member of the aceyl-CoA dehydrogenase family and metabolizes aceyl-CoA's from medium chain acyl CoA. In some embodiments, a mutation is in ACADM. In some embodiments, a mutation is in ACADM comprising a sequence as set forth in SEQ ID NO: 12. In some embodiments, a mutation in ACADM is a mutation in a peptide sequence. In some embodiments, the mutation results in a missense substitution, a nonsense substitution (*), a coding silent substitution, deletion (del), an insertion (ins), or a frameshift (fs). In some embodiments, a mutation in ACADM translates to amino acid positions in MCAD selected from: R53, Y67, I78, C116, T121, M149, T193, G195, R206, C244, S245, G267, R281, G310, M326, K329, S336, Y352, and I375, wherein the amino acids correspond to positions 53, 67, 78, 116, 121, 149, 193, 195, 206, 244, 245, 267, 281, 310, 326, 329, 336, 352, and 375 of SEQ ID NO: 23. In some embodiments, a mutation in ACADM translates to one or more different amino acid positions of SEQ ID NO: 23. In some embodiments, the mutation in ACADM, which translates to amino acid positions in MCAD, includes, but are not limited to, R53C, Y67H, I78T, 115-116del, C116Y, T121I, M149I, T193A, G195R, R206L, C244R, S245L, G267R, R281T, G310R, M326T, K329E, S336R, Y352C, and I375T. In some embodiments, the mutation in ACADM, which translates to amino acid positions in MCAD is K304E.

ACADS, also known as short-chain specific acyl-CoA dehydrogenase, encodes for the SCAD protein. SCAD is a member of the aceyl-CoA dehydrogenase family and metabolizes aceyl-CoA's from short chain acyl CoA. In some embodiments, a mutation is in ACADS. In some embodiments, a mutation is in ACADS comprising a sequence as set forth in SEQ ID NO: 13. In some embodiments, a mutation in ACADS is a mutation in a peptide sequence. In some embodiments, the mutation results in a missense substitution, a nonsense substitution (*), a coding silent substitution, deletion (del), an insertion (ins), or a frameshift (fs). In some embodiments, a mutation in ACADS translates to amino acid positions in SCAD selected from: R46, G90, G92, R107, W177, A192, R325, S353, R380, and R383, wherein the amino acids correspond to positions 46, 90, 92, 107, 177, 192, 325, 353, 380, and 383 of SEQ ID NO: 24. In some embodiments, a mutation in ACADS translates to one or more different amino acid positions of SEQ ID NO: 24. In some embodiments, the mutation in ACADS, which translates to amino acid positions in SCAD, includes, but are not limited to, R46W, G90S, G92C, 104del, R107C, W177R, A192V, R325W, S353L, R380W, and R383C.

HADHA, also known as hydroxyacyl-CoA dehydrogenase trifunctional multienzyme complex subunit alpha, encodes the protein MTPα. MTPα is a subunit of MTP, which is located at mitochondrial inner membrane and metabolizes long chain intermediates. In some embodiments, a mutation is in MTPα. In some embodiments, a mutation is in HADHA comprising a sequence as set forth in SEQ ID NO: 14. In some embodiments, a mutation in MTPα is a mutation in a peptide sequence. In some embodiments, the mutation results in a missense substitution, a nonsense substitution (*), a coding silent substitution, deletion (del), an insertion (ins), or a frameshift (fs). In some embodiments, a mutation in HADHA translates to amino acid positions in MTPα selected from: V282, I305, L341, and E510, wherein the amino acids correspond to positions 282, 305, 341, and 510 of SEQ ID NO: 25. In some embodiments, a mutation in HADHA translates to one or more different amino acid positions of SEQ ID NO: 25. In some embodiments, the mutation in HADHA, which translates to amino acid positions in MTPα, includes, but are not limited to, V282D, I305N, L341P, and E510Q. In some embodiments, the mutation in HADHA, which translates to amino acid positions in MTPα is E510Q.

HADHB, also known as hydroxyacyl-CoA dehydrogenase trifunctional multienzyme complex subunit beta, encodes the protein MTPβ. MTPβ is a subunit of MTP. In some embodiments, a mutation is in MTPβ. In some embodiments, a mutation is in HADHB comprising a sequence as set forth in SEQ ID NO: 15. In some embodiments, a mutation in MTPβ is a mutation in a peptide sequence. In some embodiments, the mutation results in a missense substitution, a nonsense substitution (*), a coding silent substitution, deletion (del), an insertion (ins), or a frameshift (fs). In some embodiments, a mutation in HADHB translates to amino acid positions in MTPβ selected from: G59, R61, R117, L121, T133, D242, R247, D263, G280, P294, G301, and R444, wherein the amino acids correspond to positions 59, 61, 117, 121, 133, 242, 247, 263, 280, 294, 301, and 444 of SEQ ID NO: 26. In some embodiments, a mutation in HADHB translates to one or more different amino acid positions of SEQ ID NO: 26. In some embodiments, the mutation in HADHB, which translates to amino acid positions in MTPβ, includes, but are not limited to, G59D, R61C, R61H, R117G, L121P, T133P, D242G, R247H, 259-270del, D263G, G280D, P294L, P294R, G301S, and R444K. In some embodiments, the mutation in HADHB, which translates to amino acid positions in MTPβ is R247C.

ECHS1, also known as enoyl-CoA hydratase, short chain, encodes the Crotonase protein, short chain protein. Crotonase functions to metabolize fatty acids during fatty acid oxidation to generate acetyl CoA. In some embodiments, a mutation is in crotonase. In some embodiments, a mutation is in ECHS1 comprising a sequence as set forth in SEQ ID NO: 16. In some embodiments, a mutation in crotonase is a mutation in a peptide sequence. In some embodiments, the mutation results in a missense substitution, a nonsense substitution (*), a coding silent substitution, deletion (del), an insertion (ins), or a frameshift (fs). In some embodiments, a mutation in ECHS1 translates to amino acid positions in crotonase selected from: A2, F33, R54, N59, I66, E77, G90, A132, A138, D150, A158, Q159, G195, C225, K273, and E281, wherein the amino acids correspond to positions 2, 33, 54, 59, 66, 77, 90, 132, 138, 150, 158, 159, 195, 225, 273, and 281 of SEQ ID NO: 27. In some embodiments, a mutation in ECHS1 translates to one or more different amino acid positions of SEQ ID NO: 27. In some embodiments, the mutation in ECHS1, which translates to amino acid positions in crotonase, includes, but are not limited to, A2V, F33S, R54H, N59S, I66T, E77Q, G90R, A132T, A138V, D150G, A158D, Q159R, G195S, C225R, K273E, and E281G.

HADH, also known as short-chain (S)-3-hydroxyacyl-CoA dehydrogenase, encodes the SCHAD protein, short chain protein. SCHAD functions in the beta oxidation of short chain fatty acids. In some embodiments, a mutation is in SCHAD. In some embodiments, a mutation is in HADH comprising a sequence as set forth in SEQ ID NO: 17. In some embodiments, a mutation in SCHAD is a mutation in a peptide sequence. In some embodiments, the mutation results in a missense substitution, a nonsense substitution (*), a coding silent substitution, deletion (del), an insertion (ins), or a frameshift (fs). In some embodiments, a mutation in HADH translates to amino acid positions in SCHAD selected from: A40, D57, and P258, wherein the amino acids correspond to positions 40, 57, and 258 of SEQ ID NO: 28. In some embodiments, a mutation in HADH translates to one or more different amino acid positions of SEQ ID NO: 28. In some embodiments, the mutation in HADH, which translates to amino acid positions in SCHAD, includes, but are not limited to, A40T, D57E, and P258L.

ACAA2, also known as medium-chain 3-ketoacyl-CoA thiolase, encodes the MCKAT protein, short chain protein. MCKAT catalyzes ketoacyl-CoA. In some embodiments, a mutation is in SCHAD. In some embodiments, a mutation is in ACAA2 comprising a sequence as set forth in SEQ ID NO: 18. In some embodiments, a mutation in MCKAT is a mutation in a peptide sequence. In some embodiments, the mutation results in a missense substitution, a nonsense substitution (*), a coding silent substitution, deletion (del), an insertion (ins), or a frameshift (fs). In some embodiments, a mutation in ACAA2 translates to one or more different amino acid positions of SEQ ID NO: 29.

ACAT1, also known as acetoacetyl-CoA thiolase or acetyl-CoA acetyltransferase 1, encodes the acetyl-CoA acetyltransferase protein. Acetyl-CoA acetyltransferase functions in ketone body metabolism. In some embodiments, a mutation is in acetoacetyl-CoA thiolase. In some embodiments, a mutation is in ACAT1 comprising a sequence as set forth in SEQ ID NO: 19. In some embodiments, a mutation in acetoacetyl-CoA thiolase is a mutation in a peptide sequence. In some embodiments, the mutation results in a missense substitution, a nonsense substitution (*), a coding silent substitution, deletion (del), an insertion (ins), or a frameshift (fs). In some embodiments, mutation in ACAT1 translates to amino acid positions in acetoacetyl-CoA thiolase selected from: N93, G152, N158, G183, T297, A301, I312, A333, G379, and A380, wherein the amino acids correspond to positions 93, 152, 158, 183, 297, 301, 312, 333, 379, and 380 of SEQ ID NO: 30. In some embodiments, a mutation in ACAT1 translates to one or more different amino acid positions of SEQ ID NO: 30. In some embodiments, the mutation in ACA TI, which translates to amino acid positions in acetoacetyl-CoA thiolase, includes, but are not limited to, 85del, N93S, G152A, N158D, G183R, T297M, A301P, I312T, A333P, G379V, and A380T.

ACADL, also known as acyl-CoA dehydrogenase long chain, encodes the LCAD protein. LCAD catalyzes the beta oxidation of straight chain fatty acids. In some embodiments, a mutation is in LCAD. In some embodiments, a mutation is in ACADL comprising a sequence as set forth in SEQ ID NO: 20. In some embodiments, a mutation in LCAD is a mutation in a peptide sequence. In some embodiments, the mutation results in a missense substitution, a nonsense substitution (*), a coding silent substitution, deletion (del), an insertion (ins), or a frameshift (fs). In some embodiments, the mutation in ACADL translates to one or more different amino acid positions of SEQ ID NO: 31.

ACAD9, also known as acyl-CoA dehydrogenase family, member 9, encodes the ACAD9 protein. ACAD9 is a member of the ACAD family that act on fatty acids comprising 14-20 carbons. In some embodiments, a mutation is in ACAD9. In some embodiments, a mutation is in ACAD9 comprising a sequence as set forth in SEQ ID NO: 21. In some embodiments, a mutation in ACAD9 is a mutation in a peptide sequence. In some embodiments, the mutation results in a missense substitution, a nonsense substitution (*), a coding silent substitution, deletion (del), an insertion (ins), or a frameshift (fs). In some embodiments, mutation in ACAD9 translates to amino acid positions in ACAD9 selected from: F44, R127, R193, A220, S234, R266, C271, G303, A326, V384, E413, R414, R417, R469W, R518, R532, and L606, wherein the amino acids correspond to positions 44, 127, 193, 220, 234, 266, 271, 303, 326, 384, 413, 414, 417, 469, 518, 532, and 606. In some embodiments, a mutation in ACAD9 translates to one or more different amino acid positions of SEQ ID NO: 32. In some embodiments, the mutation in ACAD9, which translates to amino acid positions in ACAD9, includes, but are not limited to, F441, R127K, R193W, A220V, S234F, R266Q, C271G, G303S, A326T, V384M, E413K, R414C, R417C, R469, R518H, R532W, and L606H.

Methods described herein, in some embodiments, comprise treating a FAO disorder caused by a mutation in a gene of interest, wherein the gene of interest encodes for a protein that functions as an auxiliary enzyme. Exemplary genes that encode for a protein that functions as an auxiliary enzyme include, but not limited to, ECI1, ECI2, DECR1, and ECH1. In some embodiments, the mutation is in ECI1. In some embodiments, the mutation is in ECI2. In some embodiments, the mutation is in DECR1. In some embodiments, the mutation is in ECH1. In some embodiments, the mutation is in one or more genes selected from the group consisting of ECI1, ECI2, DECR1, and ECH1.

ECI1, also known as enoyl-CoA delta isomerase 1, encodes for the protein DCI. DCI is a mitochondrial enzyme involved in beta oxidation of unsaturated fatty acids. In some embodiments, a mutation is in DCI. In some embodiments, a mutation is in ECI1 comprising a sequence as set forth in SEQ ID NO: 33. In some embodiments, a mutation in DCI is a mutation in a peptide sequence. In some embodiments, the mutation results in a missense substitution, a nonsense substitution (*), a coding silent substitution, deletion (del), an insertion (ins), or a frameshift (fs). In some embodiments, a mutation in ECI1 translates to one or more different amino acid positions of SEQ ID NO: 37.

ECI2, also known as enoyl-CoA delta isomerase 2, encodes for the protein PECI. PECI is a mitochondrial enzyme involved in beta oxidation of unsaturated fatty acids. In some embodiments, a mutation is in PECI. In some embodiments, a mutation is in ECI2 comprising a sequence as set forth in SEQ ID NO: 34. In some embodiments, a mutation in PECI is a mutation in a peptide sequence. In some embodiments, the mutation results in a missense substitution, a nonsense substitution (*), a coding silent substitution, deletion (del), an insertion (ins), or a frameshift (fs). In some embodiments, a mutation in ECI2 translates to one or more different amino acid positions of SEQ ID NO: 38.

DECR1, also known as 2,4-dienoyl-CoA reductase, encodes for the protein DECR. DECR participates in the metabolism of unsaturated fatty enoyl-CoA esters having double bonds in both even- and odd-numbered positions. In some embodiments, a mutation is in DECR. In some embodiments, a mutation is in DECR1 comprising a sequence as set forth in SEQ ID NO: 35. In some embodiments, a mutation in DECR is a mutation in a peptide sequence. In some embodiments, the mutation results in a missense substitution, a nonsense substitution (*), a coding silent substitution, deletion (del), an insertion (ins), or a frameshift (fs). In some embodiments, mutation in DECR1 translates to amino acid positions in DECR selected from: N148, Y199, S210, and K214, wherein the amino acids correspond to positions 148, 199, 210, and 214 of SEQ ID NO: 35. In some embodiments, a mutation in DECR1 translates to one or more different amino acid positions of SEQ ID NO: 39. In some embodiments, the mutation in DECR1, which translates to amino acid positions in ACAD9, includes, but are not limited to, N148A, Y199A, S210A, and K214A.

ECH1, also known as enoyl-CoA hydratase 1, encodes for the protein ECH1. ECH1 functions in the auxiliary step of the fatty acid oxidation pathway. In some embodiments, a mutation is in ECH1. In some embodiments, a mutation is in ECH1 comprising a sequence as set forth in SEQ ID NO: 36. In some embodiments, a mutation in ECH1 is a mutation in a peptide sequence. In some embodiments, the mutation results in a missense substitution, a nonsense substitution (*), a coding silent substitution, deletion (del), an insertion (ins), or a frameshift (fs). In some embodiments, a mutation in ECH1 translates to one or more different amino acid positions of SEQ ID NO: 40.

Muscle tissue is soft tissue found in most animals comprising muscle cells. Muscle cells contain protein filaments that, in some cases, slide past one another and produce a contraction that changes both the length and shape of the muscle cell. Muscles function to produce force and motion. There are three types of muscles in the body: a) skeletal muscle (the muscle responsible for moving extremities and external areas of the bodies); b) cardiac muscle (the heart muscle); and c) smooth muscle (the muscle that is in the walls of arteries and bowel).

The term “muscle cell” as used herein refers to any cell that contributes to muscle tissue. Myoblasts, satellite cells, myotubes, and myofibril tissues are all included in the term “muscle cells” and, in some embodiments, are treated using the methods described herein. Muscle cell effects, in some cases, are induced within skeletal, cardiac, and smooth muscles.

Skeletal muscle, or voluntary muscle, is generally anchored by tendons to bone and is generally used to effect skeletal movement such as locomotion or in maintaining posture. Although some control of skeletal muscle is generally maintained as an unconscious reflex (e.g., postural muscles or the diaphragm), skeletal muscles react to conscious control. Smooth muscle, or involuntary muscle, is found within the walls of organs and structures such as the esophagus, stomach, intestines, uterus, urethra, and blood vessels. Unlike skeletal muscle, smooth muscle is not under conscious control. Cardiac muscle is also an involuntary muscle but more closely resembles skeletal muscle in structure and is found only in the heart. Cardiac and skeletal muscles are striated in that they contain sarcomeres that are packed into highly regular arrangements of bundles. By contrast, the myofibrils of smooth muscle cells are not arranged in sarcomeres and therefore are not striated.

Skeletal muscle is further divided into two broad types: Type I (or “slow twitch”) and Type II (or “fast twitch”). Type I muscle fibers are dense with capillaries and are rich in mitochondria and myoglobin, which gives Type I muscle tissue a characteristic red color. Type I muscle fibers, in some cases, carry more oxygen and sustain aerobic activity using fats or carbohydrates for fuel. Type I muscle fibers contract for long periods of time but with little force. Type II muscle fibers are subdivided into three major subtypes (IIa, IIx, and IIb) that vary in both contractile speed and force generated. Type II muscle fibers contract quickly and powerfully but fatigue very rapidly, and therefore produce only short, anaerobic bursts of activity before muscle contraction becomes painful.

Mitochondrial biogenesis is measured by mitochondrial mass and volume through histological section staining using a fluorescently labeled antibody specific to the oxidative-phosphorylation complexes, such as the Anti-OxPhox Complex Vd subunit antibody from Life Technologies or using mitochondrial specific dyes in live cell staining, such as the Mito-tracker probes from Life Technologies. Mitochondrial biogenesis, in some cases, is also measured by monitoring the gene expression of one or more mitochondrial biogenesis related transcription factors such as PGC1α, NRF1, or NRF2 using a technique such as QPCR.

In some aspects, PPARδ agonist is administered in a therapeutically effective amount to a subject (e.g., a human). As used herein, the term “effective amount” or “therapeutically effective amount” refers to an amount of an active ingredient that elicits the desired biological or medicinal response, for example, reduction or alleviation of the symptoms of the condition being treated. In some embodiments of the invention, the amount of PPARδ agonist administered varies depending on various factors, including, but not limited to, the weight of the subject, the nature and/or extent of the subject's condition, etc.

### Compounds

A peroxisome proliferator activated receptor-delta (PPARS) agonist compound is a fatty acid, lipid, protein, peptide, small molecule, or other chemical entity that binds to the cellular PPARδ and elicits a downstream response, namely gene transcription, either native gene transcription or a reporter construct gene transcription, comparable to endogenous ligands such as retinoic acid or comparable to a standard reference PPARδ agonist such as carbacyclin.

In an embodiment, a PPARδ agonist is a selective agonist. As used herein, a selective PPARδ agonist is viewed as a chemical entity that binds to and activates the cellular PPARδ and does not substantially activate the cellular peroxisome proliferator activated receptors alpha (PPARα) and gamma (PPARγ). As used herein, a selective PPARδ agonist is a chemical entity that has at least a 10-fold maximum activation (as compared to endogenous receptor ligand) with a greater than 100-fold potency for activation of PPARδ relative to either or both of PPARα and PPARγ. In a further embodiment, a selective PPARδ agonist is a chemical entity that binds to and activates the cellular human PPARδ and does not substantially activate either or both of human PPARα and PPARγ. In a further embodiment, a selective PPARδ agonist is a chemical entity that has at least about a 10-fold, or about a 20-fold, or about a 30-fold, or about a 40-fold, or about a 50-fold, or about a 100-fold potency for activation of PPARδ relative to either or both of PPARα and PPARγ.

In some embodiments, a selective PPARδ agonist compound contemplated herein is capable of simultaneously contacting the amino-acid residues at positions VAL312, and ILE328 of PPARδ (hPPARδ numbering). In some embodiments, a selective PPARδ agonist compound is capable of simultaneously contacting the amino-acid residues at positions VAL298, LEU303, VAL312, and ILE328 (hPPARδ numbering).

“Activation” herein is defined as the abovementioned downstream response, which in the case of PPAR's is gene transcription. Gene transcription, in some cases, is measured indirectly as downstream production of proteins reflective of the activation of the particular PPAR subtype under study. Alternatively, an artificial reporter construct, in some cases, is employed to study the activation of the individual PPAR's expressed in cells. The ligand binding domain of the particular receptor to be studied, in some embodiments, is fused to the DNA binding domain of a transcription factor, which produces convenient laboratory readouts, such as the yeast GAL4 transcription factor DNA binding domain. The fusion protein, in some cases, is transfected into a laboratory cell line along with a Gal4 enhancer, which effects the expression of the luciferase protein. When such a system is transfected into a laboratory cell line, binding of a receptor agonist to the fusion protein will result in light emission.

A selective PPARδ agonist, in some embodiments, exemplifies the above gene transcription profile in cells selectively expressing PPARS, and not in cells selectively expressing PPARγ or PPARα. In an embodiment, the cells express human PPARδ, PPARγ, and PPARα, respectively.

In a further embodiment, a PPARδ agonist has an EC₅₀ value of less than about 5 μm as determined by the PPAR transient transactivation assay described below. In an embodiment, the EC₅₀ value is less than about 1 μm. In another embodiment, the EC₅₀ value is less than about 500 nM. In another embodiment, the EC₅₀ value is less than about 100 nM. In another embodiment, the EC₅₀ value is less than about 50 nM.

The PPAR transient transactivation assay, in some cases, is based on transient transfection into human HEK293 cells of two plasmids encoding a chimeric test protein and a reporter protein respectively. The chimeric test protein, in some cases, is a fusion of the DNA binding domain (DBD) from the yeast GAL4 transcription factor to the ligand binding domain (LBD) of the human PPAR proteins. The PPAR-LBD moiety harbored in addition to the ligand binding pocket also has the native activation domain, allowing the fusion protein to function as a PPAR ligand dependent transcription factor. The GAL4 DBD will direct the chimeric protein to bind only to Gal4 enhancers (of which none existed in HEK293 cells). The reporter plasmid contained a Gal4 enhancer driving the expression of the firefly luciferase protein. After transfection, HEK293 cells expressed the GAL4-DBD-PPAR-LBD fusion protein. The fusion protein will in turn bind to the Gal4 enhancer controlling the luciferase expression, and do nothing in the absence of ligand. Upon addition to the cells of a PPAR ligand, luciferase protein will be produced in amounts corresponding to the activation of the PPAR protein. The amount of luciferase protein is measured by light emission after addition of the appropriate substrate.

Cell Culture and Transfection: HEK293 cells, in some cases, are grown in DMEM+10% FCS. Cells, in some cases, are seeded in 96-well plates the day before transfection to give a confluency of 50-80% at transfection. A total of 0.8 mg DNA containing 0.64 mg pM1a/gLBD, 0.1 mg pCMVbGal, 0.08 mg pGL2(Gal4)₅, and 0.02 mg pADVANTAGE, in some cases, are transfected per well using FuGene transfection reagent according to the manufacturer's instructions. Cells, in some instances, are allowed to express protein for 48 hours followed by addition of compound.

Plasmids: Human PPARδ, in some cases, is obtained by PCR amplification using cDNA synthesized by reverse transcription of mRNA from human liver, adipose tissue, and plancenta, respectively. In some embodiments, amplified cDNAs is cloned into pCR2.1 and sequenced. The ligand binding domain (LBD) of each PPAR isoform, in some cases, is generated by PCR (PPARδ: aa 128-C-terminus) and fused to the DNA binding domain (DBD) of the yeast transcription factor GAL4 by subcloning fragments in frame into the vector pM1 (Sadowski et al. (1992), Gene 118, 137), generating the plasmids pM1αLBD, pM1γLBD, and pM16. Ensuing fusions, in some cases, is verified by sequencing. The reporter, in some cases, is constructed by inserting an oligonucleotide encoding five repeats of the GAL4 recognition sequence (Webster et al. (1988), Nucleic Acids Res. 16, 8192) into the vector pGL2 promotor (Promega), generating the plasmid pGL2(GAL4)₅. pCMVbGal, in some cases, is purchased from Clontech and pADVANTAGE, in some cases, is purchased from Promega.

Compounds: Compounds, in some cases, are dissolved in DMSO and diluted 1:1000 upon addition to the cells. Compounds, in some cases, are tested in quadruple in concentrations ranging from 0.001 to 300 μM. Cells, in some cases, are treated with compound for 24 h followed by luciferase assay. Each compound, in some cases, is tested in at least two separate experiments.

Luciferase assay: Medium including test compound, in some cases, is aspirated and 100 μl PBS including 1 mM Mg⁺⁺ and Ca⁺⁺, in some cases, is added to each well. In some embodiments, the luciferase assay is performed using the LucLite kit according to the manufacturer's instructions (Packard Instruments). Light emission, in some cases, is quantified by counting on a Packard LumiCounter. To measure β-galactosidase activity, 25 ml supernatant from each transfection lysate, in some cases, is transferred to a new microplate. In some embodiments, β-Galactosidase assays are performed in the microwell plates using a kit from Promega and read in a Labsystems Ascent Multiscan reader. The β-galactosidase data, in some cases, is used to normalize (transfection efficiency, cell growth, etc.) the luciferase data.

Statistical methods: The activity of a compound, in some cases, is calculated as fold induction compared to an untreated sample. In some embodiments, for each compound, the efficacy (maximal activity) is given as a relative activity compared to Wy14,643 for PPARα, rosiglitazone for PPARγ, and carbacyclin for PPARδ. The EC₅₀ is the concentration giving 50% of maximal observed activity. EC₅₀ values, in some cases, is calculated via non-linear regression using GraphPad PRISM 3.02 (GraphPad Software, San Diego, Calif.).

In a further embodiment, a PPARδ agonist has a molecular weight of less than about 1000 g/mol, or a molecular weight of less than about 950 g/mol, or a molecular weight of less than about 900 g/mol, or a molecular weight of less than about 850 g/mol, or a molecular weight of less than about 800 g/mol, or a molecular weight of less than about 750 g/mol, or a molecular weight of less than about 700 g/mol, or a molecular weight of less than about 650 g/mol, or a molecular weight of less than about 600 g/mol, or a molecular weight of less than about 550 g/mol, or a molecular weight of less than about 500 g/mol, or a molecular weight of less than about 450 g/mol, or a molecular weight of less than about 400 g/mol, or a molecular weight of less than about 350 g/mol, or a molecular weight of less than about 300 g/mol, or a molecular weight of less than about 250 g/mol. In another embodiment, a PPARδ agonist has a molecular weight of greater than about 200 g/mol, or a molecular weight of greater than about about 250 g/mol, or a molecular weight of greater than about 250 g/mol, or a molecular weight of greater than about 300 g/mol, or a molecular weight of greater than about 350 g/mol, or a molecular weight of greater than about 400 g/mol, or a molecular weight of greater than about 450 g/mol, or a molecular weight of greater than about 500 g/mol, or a molecular weight of greater than about 550 g/mol, or a molecular weight of greater than about 600 g/mol, or a molecular weight of greater than about 650 g/mol, or a molecular weight of greater than about 700 g/mol, or a molecular weight of greater than about 750 g/mol, or a molecular weight of greater than about 800 g/mol, or a molecular weight of greater than about 850 g/mol, or a molecular weight of greater than about 900 g/mol, or a molecular weight of greater than about 950 g/mol, or a molecular weight of greater than about 1000 g/mol. Any of the upper and lower limits described above in this paragraph, in some embodiments, are combined.

In some embodiments, a PPARδ agonist is a PPARδ agonist compound disclosed in any of the following published patent applications: WO 97/027847, WO 97/027857, WO 97/028115, WO 97/028137, WO 97/028149, WO 98/027974, WO 99/004815, WO 2001/000603, WO 2001/025181, WO 2001/025226, WO 2001/034200, WO 2001/060807, WO 2001/079197, WO 2002/014291, WO 2002/028434, WO 2002/046154, WO 2002/050048, WO 2002/059098, WO 2002/062774, WO 2002/070011, WO 2002/076957, WO 2003/016291, WO 2003/024395, WO 2003/033493, WO 2003/035603, WO 2003/072100, WO 2003/074050, WO 2003/074051, WO 2003/074052, WO 2003/074495, WO 2003/084916, WO 2003/097607, WO 2004/000315, WO 2004/000762, WO 2004/005253, WO 2004/037776, WO 2004/060871, WO 2004/063165, WO 2004/063166, WO 2004/073606, WO 2004/080943, WO 2004/080947, WO 2004/092117, WO 2004/092130, WO 2004/093879, WO 2005/060958, WO 2005/097098, WO 2005/097762, WO 2005/097763, WO 2005/115383, WO 2006/055187, WO 2007/003581, and WO 2007/071766 (each of which is incorporated for such PPARδ agonist compounds).

In some embodiments, a PPARδ agonist is a PPARδ agonist compound disclosed in any of the following published patent applications: WO2014/165827; WO2016/057660; WO2016/057658; WO2017/180818; WO2017/062468; and WO/2018/067860 (each of which is incorporated for such PPARδ agonist compounds).

In some embodiments, a PPARδ agonist is a PPARδ agonist compound disclosed in any of the following published patent applications: United States Patent Application Publication Nos. 20160023991, 20170226154, 20170304255, and 20170305894 (each of which is incorporated for such PPARδ agonist compounds).

In some embodiments, a PPARδ agonist compound is a phenoxyalkylcarboxylic acid compound. In some embodiments, the phenoxyalkylcarboxylic acid compound is a 2-methylphenoxyalkylcarboxylic acid compound.

In some embodiments, a PPARδ agonist compound is a phenoxyalkylcarboxylic acid compound that is a phenoxyethanoic acid compound, phenoxypropanoic acid compound, phenoxypropenoic acid compound, phenoxybutanoic acid compound, phenoxybutenoic acid compound, phenoxypentanoic acid compound, phenoxypentenoic acid compound, phenoxyhexanoic acid compound, phenoxyhexenoic acid compound, phenoxyoctanoic acid compound, phenoxyoctenoic acid compound, phenoxynonanoic acid compound, phenoxynonenoic acid compound, phenoxydecanoic acid compound, or phenoxydecenoic acid compound. In some embodiments, a PPARδ agonist compound is a phenoxyethanoic acid compound or a phenoxyhexanoic acid compound. In some embodiments, a PPARδ agonist compound is a phenoxyethanoic acid compound. In some embodiments, the phenoxyethanoic acid compound is a 2-methylphenoxyethanoic acid compound. In some embodiments, a PPARδ agonist compound is a phenoxyhexanoic acid compound.

In some embodiments, a PPARδ agonist compound is a phenoxyethanoic acid compound, a ((benzamidomethyl)phenoxy)hexanoic acid compound, a ((heteroarylmethyl)phenoxy)hexanoic acid compound, a methylthiophenoxyethanoic acid compound, or an allyloxyphenoxyethanoic acid acid compound.

In some embodiments, a PPARδ agonist compound is a ((benzamidomethyl)phenoxy)hexanoic acid compound.

In some embodiments, a PPARδ agonist compound is a ((heteroarylmethyl)phenoxy)hexanoic acid compound. In some embodiments, a PPARδ agonist compound is a ((imidazolylmethyl)phenoxy)hexanoic acid compound. In some embodiments, a PPARδ agonist compound is an imidazol-1-ylmethylphenoxyhexanoic acid compound. In some embodiments, a PPARδ agonist compound is a 6-(2-((2-phenyl-1H-imidazol-1-yl)methyl)phenoxy)hexanoic acid.

In some embodiments, a PPARδ agonist compound is an allyloxyphenoxyethanoic acid compound. In some embodiments, the allyloxyphenoxyethanoic acid compound is a 4-allyloxy-2-methylphenoxy)ethanoic acid compound.

In some embodiments, a PPARδ agonist compound is a methylthiophenoxyethanoic acid compound. In some embodiments, a PPARδ agonist compound is a 4-(methylthio)phenoxy)ethanoic acid compound.

In some embodiments, a PPARδ agonist compound is a phenoxyalkylcarboxylic acid compound selected from the group consisting of: (Z)-[2-Methyl-4-[3-(4-methylphenyl)-3-[4-[3-(morpholin-4-yl)propynyl]phenyl]allyloxy]-phenoxy]acetic acid; (E)-[2-Methyl-4-[3-[4-[3-(pyrazol-1-yl)prop-1-ynyl]phenyl]-3-(4-trifluoromethylphenyl)-allyloxy]phenoxy]acetic acid; (E)-[4-[3-(4-Fluorophenyl)-3-[4-[3-(morpholin-4-yl)propynyl]phenyl]allyloxy]-2-methyl-phenoxy]acetic acid (Compound 1); (E)-[2-Methyl-4-[3-[4-[3-(morpholin-4-yl)propynyl]phenyl]-3-(4-trifluoromethylphenyl)allyloxy]-phenoxy]acetic acid; (E)-[4-[3-(4-Chlorophenyl)-3-[4-[3-(morpholin-4-yl)propynyl]phenyl]allyloxy]-2-methyl-phenoxy]acetic acid; (E)-[4-[3-(4-Chlorophenyl)-3-[4-[3-(morpholin-4-yl)propynyl]phenyl]allyloxy]-2-methylphenyl]-propionic acid; {4-[3-Isobutoxy-5-(3-morpholin-4-yl-prop-1-ynyl)-benzylsulfanyl]-2-methyl-phenoxy}-acetic acid; {4-[3-Isobutoxy-5-(3-morpholin-4-yl-prop-1-ynyl)-phenylsulfanyl]-2-methyl-phenoxy}-acetic acid; and {4-[3,3-Bis-(4-bromo-phenyl)-allyloxy]-2-methyl-phenoxy}-acetic acid; (R)-3-methyl-6-(2-((5-methyl-2-(4-(trifluoromethyl)phenyl)-1H-imidazol-1-yl)methyl)phenoxy)hexanoic acid; (R)-3-methyl-6-(2-((5-methyl-2-(6-(trifluoromethyl)pyridin-3-yl)-1H-imidazol-1-yl)methyl)phenoxy)hexanoic acid; (E)-[4-[3-(4-Fluorophenyl)-3-[4-[3-(morpholin-4-yl)propynyl]phenyl]allyloxy]-2-methyl-phenoxy]acetic acid (Compound 1); 2-{4-[({2-[2-Fluoro-4-(trifluoromethyl)phenyl]-4-methyl-1,3-thiazol-5-yl}methyl)sulfanyl]-2-methylphenoxy}-2-methylpropanoic acid (sodelglitazar; GW677954); 2-[2-methyl-4-[[3-methyl-4-[[4-(trifluoromethyl)phenyl]methoxy]phenyl]thio]phenoxy]-acetic acid; 2-[2-methyl-4-[[[4-methyl-2-[4-(trifluoromethyl)phenyl]-5-thiazolyl]methyl]thio]phenoxy]-acetic acid (GW-501516); [4-[[[2-[3-Fluoro-4-(trifluoromethyl)phenyl]-4-methyl-5-thiazolyl]methyl]thio]-2-methylphenoxy]acetic acid (GW0742 also known as GW610742); 2-[2,6 dimethyl-4-[3-[4-(methylthio)phenyl]-3-oxo-1(E)-propenyl]phenoxyl]-2-methylpropanoic acid (elafibranor; GFT-505); {2-methyl-4-[5-methyl-2-(4-trifluoromethyl-phenyl)-2H-[1,2,3]triazol-4-ylmethylsulfanyl]-phenoxy}-acetic acid; and [4-({(2R)-2-Ethoxy-3-[4-(trifluoromethyl)phenoxy]propyl}sulfanyl)-2-methylphenoxy]acetic acid (seladelpar; MBX-8025); (S)-4-[cis-2,6-dimethyl-4-(4-trifluoromethoxy-phenyl)piperazine-1-sulfonyl]-indan-2-carboxylic acid or a tosylate salt thereof (KD-3010); (2s)-2-{4-butoxy-3-[({[2-Fluoro-4-(Trifluoromethyl)phenyl]carbonyl}amino)methyl]benzyl}butanoic acid (TIPP-204); [4-[3-(4-Acetyl-3-hydroxy-2-propylphenoxy)propoxy]phenoxy]acetic acid (L-165,0411); 2-(4-{2-[(4-Chlorobenzoyl)amino]ethyl}phenoxy)-2-methylpropanoic acid (bezafibrate); or a pharmaceutically acceptable salt thereof.

In another embodiment, a PPARδ agonist is a 2-methylphenoxyalkylcarboxylic acid compound selected from the group consisting of (E)-[4-[3-(4-Fluorophenyl)-3-[4-[3-(morpholin-4-yl)propynyl]phenyl]allyloxy]-2-methyl-phenoxy]acetic acid (Compound 1); 2-{4-[({2-[2-Fluoro-4-(trifluoromethyl)phenyl]-4-methyl-1,3-thiazol-5-yl}methyl)sulfanyl]-2-methylphenoxy}-2-methylpropanoic acid (sodelglitazar; GW677954); 2-[2-methyl-4-[[3-methyl-4-[[4-(trifluoromethyl)phenyl]methoxy]phenyl]thio]phenoxy]-acetic acid; 2-[2-methyl-4-[[[4-methyl-2-[4-(trifluoromethyl)phenyl]-5-thiazolyl]methyl]thio]phenoxy]-acetic acid (GW-501516); [4-[[[2-[3-Fluoro-4-(trifluoromethyl)phenyl]-4-methyl-5-thiazolyl]methyl]thio]-2-methylphenoxy]acetic acid (GW0742 also known as GW610742); 2-[2,6 dimethyl-4-[3-[4-(methylthio)phenyl]-3-oxo-1(E)-propenyl]phenoxyl]-2-methylpropanoic acid (elafibranor; GFT-505); {2-methyl-4-[5-methyl-2-(4-trifluoromethyl-phenyl)-2H-[1,2,3]triazol-4-ylmethylsulfanyl]-phenoxy}-acetic acid; and [4-({(2R)-2-Ethoxy-3-[4-(trifluoromethyl)phenoxy]propyl}sulfanyl)-2-methylphenoxy]acetic acid (seladelpar; MBX-8025).

In another embodiment, a PPARδ agonist is a compound selected from the group consisting of (S)-4-[cis-2,6-dimethyl-4-(4-trifluoromethoxy-phenyl)piperazine-1-sulfonyl]-indan-2-carboxylic acid or a tosylate salt thereof (KD-3010); (2s)-2-{4-butoxy-3-[({[2-Fluoro-4-(Trifluoromethyl)phenyl]carbonyl}amino)methyl]benzyl}butanoic acid (TIPP-204); [4-[3-(4-Acetyl-3-hydroxy-2-propylphenoxy)propoxy]phenoxy]acetic acid (L-165,0411); and 2-(4-{2-[(4-Chlorobenzoyl)amino]ethyl}phenoxy)-2-methylpropanoic acid (bezafibrate).

In another embodiment, a PPARδ agonist is a compound selected from the group consisting of sodelglitazar; lobeglitazone; netoglitazone; and isaglitazone; 2-(4-{2-[(4-Chlorobenzoyl)amino]ethyl}phenoxy)-2-methylpropanoic acid (bezafibrate); 2-[2-methyl-4-[[3-methyl-4-[[4-(trifluoromethyl)phenyl]methoxy]phenyl]thio]phenoxy]-acetic acid (See WO 2003/024395); (S)-4-[cis-2,6-dimethyl-4-(4-trifluoromethoxy-phenyl)piperazine-1-sulfonyl]-indan-2-carboxylic acid or a tosylate salt thereof (KD-3010); 4-butoxy-a-ethyl-3-[[[2-fluoro-4-(trifluoromethyl)benzoyl]amino]methyl]-benzenepropanoic acid (TIPP-204); 2-[2-methyl-4-[[[4-methyl-2-[4-(trifluoromethyl)phenyl]-5-thiazolyl]methyl]thio]phenoxy]-acetic acid (GW-501516); 2-[2,6 dimethyl-4-[3-[4-(methylthio)phenyl]-3-oxo-1(E)-propenyl]phenoxyl]-2-methylpropanoic acid (GFT-505); {2-methyl-4-[5-methyl-2-(4-trifluoromethyl-phenyl)-2H-[1,2,3]triazol-4-ylmethylsylfanyl]-phenoxy}-acetic acid; and [4-({(2R)-2-Ethoxy-3-[4-(trifluoromethyl)phenoxy]propyl}sulfanyl)-2-methylphenoxy]acetic acid (seladelpar; MBX-8025).

In some embodiments, a PPARδ agonist is (E)-[4-[3-(4-Fluorophenyl)-3-[4-[3-(morpholin-4-yl)propynyl]phenyl]allyloxy]-2-methyl-phenoxy]acetic acid (Compound 1):

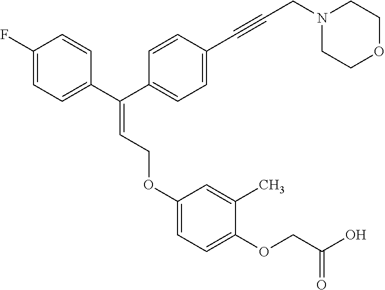 [SMILES: Cc1cc(OC/C=C(/c2ccc(F)cc2)c2ccc(C#CCN3CCOCC3)cc2)ccc1OCC(=O)O]

An example of the chemical synthesis of (E)-[4-[3-(4-Fluorophenyl)-3-[4-[3-(morpholin-4-yl)propynyl]phenyl]allyloxy]-2-methyl-phenoxy]acetic acid is found in Example 10 of PCT Application Pub. No. WO 2007/071766.

Compound 1 was tested on all three human PPAR subtypes (hPPAR): hPPARα, hPPARγ, and hPPARδ in vitro assays testing for such activity. Compound 1 exhibited a significantly greater selectivity for PPARδ over PPARα and PPARγ (by at least about 100-fold and at least about 400-fold, respectively). In some cases, Compound 1 acts as a full agonist of PPARδ and only a partial agonist for both PPARα and PPARγ. In some cases, Compound 1 demonstrates negligible activity on PPARα and/or PPARγ in tranasctivation assays testing for such activity.

In some embodiments, Compound 1 did not show any human retinoid X receptor (hRXR) activity, or activity on the nuclear receptors FXR, LXRα or LXRβ. as a full agonist of PPARδ and only a partial agonist for both PPARα and PPARγ.

In some embodiments, a PPARδ agonist is (Z)-[2-Methyl-4-[3-(4-methylphenyl)-3-[4-[3-(morpholin-4-yl)propynyl]phenyl]allyloxy]-phenoxy]acetic acid:

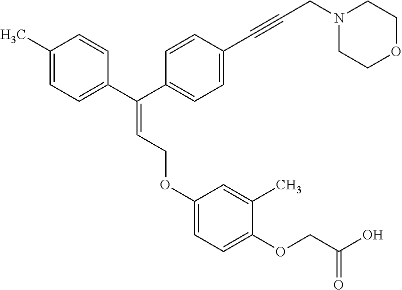 [SMILES: Cc1ccc(/C(=C/COc2ccc(OCC(=O)O)c(C)c2)c2ccc(C#CCN3CCOCC3)cc2)cc1]

An example of the chemical synthesis of (Z)-[2-Methyl-4-[3-(4-methylphenyl)-3-[4-[3-(morpholin-4-yl)propynyl]phenyl]allyloxy]-phenoxy]acetic acid is found in Example 3 of PCT Application Pub. No. WO 2007/071766.

In some embodiments, a PPARδ agonist is (E)-[2-Methyl-4-[3-[4-[3-(pyrazol-1-yl)prop-1-ynyl]phenyl]-3-(4-trifluoromethylphenyl)-allyloxy]phenoxy]acetic acid:

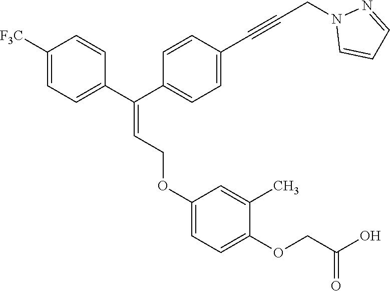 [SMILES: Cc1cc(OC/C=C(\c2ccc(C#CCn3cccn3)cc2)c2ccc(C(F)(F)F)cc2)ccc1OCC(=O)O]

An example of the chemical synthesis of (E)-[2-Methyl-4-[3-[4-[3-(pyrazol-1-yl)prop-1-ynyl]phenyl]-3-(4-trifluoromethylphenyl)-allyloxy]phenoxy]acetic acid is found in Example 4 of PCT Application Pub. No. WO 2007/071766.

In some embodiments, a PPARδ agonist is (E)-[2-Methyl-4-[3-[4-[3-(morpholin-4-yl)propynyl]phenyl]-3-(4-trifluoromethylphenyl)allyloxy]-phenoxy]acetic acid:

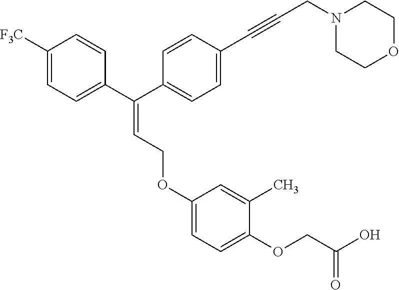 [SMILES: Cc1cc(OC/C=C(\c2ccc(C#CCN3CCOCC3)cc2)c2ccc(C(F)(F)F)cc2)ccc1OCC(=O)O]

An example of the chemical synthesis of (E)-[2-Methyl-4-[3-[4-[3-(morpholin-4-yl)propynyl]phenyl]-3-(4-trifluoromethylphenyl)allyloxy]-phenoxy]acetic acid is found in Example 20 of PCT Application Pub. No. WO 2007/071766.

In some embodiments, a PPARδ agonist is (E)-[4-[3-(4-Chlorophenyl)-3-[4-[3-(morpholin-4-yl)propynyl]phenyl]allyloxy]-2-methyl-phenoxy]acetic acid:

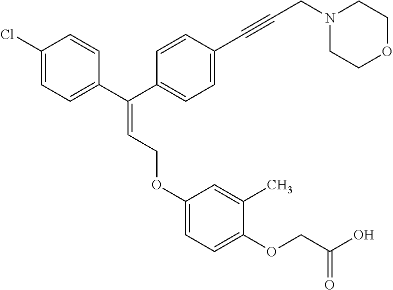 [SMILES: Cc1cc(OC/C=C(/c2ccc(Cl)cc2)c2ccc(C#CCN3CCOCC3)cc2)ccc1OCC(=O)O]

An example of the chemical synthesis of (E)-[4-[3-(4-Chlorophenyl)-3-[4-[3-(morpholin-4-yl)propynyl]phenyl]allyloxy]-2-methyl-phenoxy]acetic acid is found in Example 46 of PCT Application Pub. No. WO 2007/071766.

In some embodiments, a PPARδ agonist is (E)-[4-[3-(4-Chlorophenyl)-3-[4-[3-(morpholin-4-yl)propynyl]phenyl]allyloxy]-2-methylphenyl]-propionic acid:

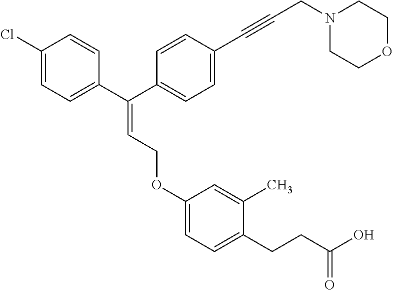 [SMILES: Cc1cc(OC/C=C(/c2ccc(Cl)cc2)c2ccc(C#CCN3CCOCC3)cc2)ccc1CCC(=O)O]

An example of the chemical synthesis of (E)-[4-[3-(4-Chlorophenyl)-3-[4-[3-(morpholin-4-yl)propynyl]phenyl]allyloxy]-2-methylphenyl]-propionic acid is found in Example 63 of PCT Application Pub. No. WO 2007/071766.

In some embodiments, a PPARδ agonist is {4-[3,3-Bis-(4-bromo-phenyl)-allyloxy]-2-methyl-phenoxy}-acetic acid:

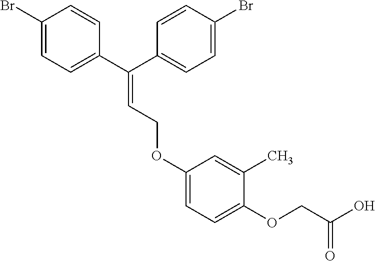 [SMILES: Cc1cc(OCC=C(c2ccc(Br)cc2)c2ccc(Br)cc2)ccc1OCC(=O)O]

An example of the chemical synthesis of {4-[3,3-Bis-(4-bromo-phenyl)-allyloxy]-2-methyl-phenoxy}-acetic acid is found in Example 10 of PCT Application Pub. No. WO 2004/037776.

In some embodiments, a PPARδ agonist is {4-[3-Isobutoxy-5-(3-morpholin-4-yl-prop-1-ynyl)-benzylsulfanyl]-2-methyl-phenoxy}-acetic acid:

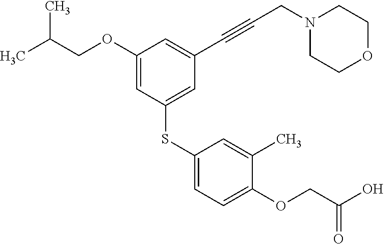 [SMILES: Cc1cc(Sc2cc(C#CCN3CCOCC3)cc(OCC(C)C)c2)ccc1OCC(=O)O]

An example of the chemical synthesis of {4-[3-Isobutoxy-5-(3-morpholin-4-yl-prop-1-ynyl)-benzylsulfanyl]-2-methyl-phenoxy}-acetic acid is found in Example 9 of PCT Application Pub. No. WO 2007/003581.

In some embodiments, a PPARδ agonist is {4-[3-Isobutoxy-5-(3-morpholin-4-yl-prop-1-ynyl)-phenylsulfanyl]-2-methyl-phenoxy}-acetic acid:

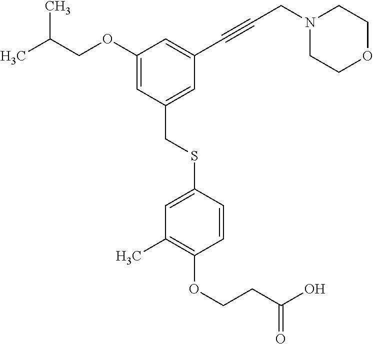 [SMILES: Cc1cc(SCc2cc(C#CCN3CCOCC3)cc(OCC(C)C)c2)ccc1OCCC(=O)O]

An example of the chemical synthesis of {4-[3-Isobutoxy-5-(3-morpholin-4-yl-prop-1-ynyl)-phenylsulfanyl]-2-methyl-phenoxy}-acetic acid is found in Example 35 of PCT Application Pub. No. WO 2007/003581.

Accordingly, in an embodiment, a PPARδ agonist is a compound selected from the group consisting of: (Z)-[2-Methyl-4-[3-(4-methylphenyl)-3-[4-[3-(morpholin-4-yl)propynyl]phenyl]allyloxy]-phenoxy]acetic acid; (E)-[2-Methyl-4-[3-[4-[3-(pyrazol-1-yl)prop-1-ynyl]phenyl]-3-(4-trifluoromethylphenyl)-allyloxy]phenoxy]acetic acid; (E)-[4-[3-(4-Fluorophenyl)-3-[4-[3-(morpholin-4-yl)propynyl]phenyl]allyloxy]-2-methyl-phenoxy]acetic acid; (E)-[2-Methyl-4-[3-[4-[3-(morpholin-4-yl)propynyl]phenyl]-3-(4-trifluoromethylphenyl)allyloxy]-phenoxy]acetic acid; (E)-[4-[3-(4-Chlorophenyl)-3-[4-[3-(morpholin-4-yl)propynyl]phenyl]allyloxy]-2-methyl-phenoxy]acetic acid; (E)-[4-[3-(4-Chlorophenyl)-3-[4-[3-(morpholin-4-yl)propynyl]phenyl]allyloxy]-2-methylphenyl]-propionic acid; {4-[3-Isobutoxy-5-(3-morpholin-4-yl-prop-1-ynyl)-benzylsulfanyl]-2-methyl-phenoxy}-acetic acid; {4-[3-Isobutoxy-5-(3-morpholin-4-yl-prop-1-ynyl)-phenylsulfanyl]-2-methyl-phenoxy}-acetic acid; and {4-[3,3-Bis-(4-bromo-phenyl)-allyloxy]-2-methyl-phenoxy}-acetic acid; or a pharmaceutically acceptable salt thereof.

In a further embodiment, a PPARδ agonist is (E)-[4-[3-(4-Fluorophenyl)-3-[4-[3-(morpholin-4-yl)propynyl]phenyl]allyloxy]-2-methyl-phenoxy]acetic acid or a pharmaceutically acceptable salt thereof. In some embodiments, the PPARδ agonist is (E)-[4-[3-(4-Fluorophenyl)-3-[4-[3-(morpholin-4-yl)propynyl]phenyl]allyloxy]-2-methyl-phenoxy]acetic acid sodium salt.

In a further embodiment, a PPARδ agonist is Compound 1, Compound 2, Compound 3, Compound 4, Compound 5, Compound 6, Compound 7, Compound 8, Compound 9, Compound 10, Compound 11, Compound 12, Compound 13, Compound 14, Compound 15, or Compound 16, disclosed in Wu et al. Proc Natl Acad Sci USA Mar. 28, 2017 114 (13) E2563-E2570.

In a further embodiment, a PPARδ agonist is (R)-3-methyl-6-(2-((5-methyl-2-(4-(trifluoromethyl)phenyl)-1H-imidazol-1-yl)methyl)phenoxy)hexanoic acid, or (R)-3-methyl-6-(2-((5-methyl-2-(6-(trifluoromethyl)pyridin-3-yl)-1H-imidazol-1-yl)methyl)phenoxy)hexanoic acid, or a pharmaceutically acceptable salt thereof.

In a further embodiment, a PPARδ agonist is (R)-3-methyl-6-(2-((5-methyl-2-(4-(trifluoromethyl)phenyl)-1H-imidazol-1-yl)methyl)phenoxy)hexanoic acid, or a pharmaceutically acceptable salt thereof. In some embodiments, the PPARδ agonist is the hemisulfate salt of (R)-3-methyl-6-(2-((5-methyl-2-(4-(trifluoromethyl)phenyl)-1H-imidazol-1-yl)methyl)phenoxy)hexanoic acid. In some embodiments, the PPARδ agonist is the meglumine salt of (R)-3-methyl-6-(2-((5-methyl-2-(4-(trifluoromethyl)phenyl)-1H-imidazol-1-yl)methyl)phenoxy)hexanoic acid.

In a further embodiment, a PPARδ agonist is (R)-3-methyl-6-(2-((5-methyl-2-(6-(trifluoromethyl)pyridin-3-yl)-1H-imidazol-1-yl)methyl)phenoxy)hexanoic acid, or a pharmaceutically acceptable salt thereof. In some embodiments, the PPARδ agonist is the hemisulfate salt of (R)-3-methyl-6-(2-((5-methyl-2-(6-(trifluoromethyl)pyridin-3-yl)-1H-imidazol-1-yl)methyl)phenoxy)hexanoic acid. In some embodiments, the PPARδ agonist is the meglumine salt of (R)-3-methyl-6-(2-((5-methyl-2-(6-(trifluoromethyl)pyridin-3-yl)-1H-imidazol-1-yl)methyl)phenoxy)hexanoic acid.

In a further embodiment, a PPARδ agonist is 2-(2-methyl-4-(((2-(4-(trifluoromethyl)phenyl)-2H-1,2,3-triazol-4-yl)methyl)thio)phenoxy)acetic acid, or a pharmaceutically acceptable salt thereof.

In a further embodiment, a PPARδ agonist is (R)-2-(4-((2-ethoxy-3-(4-(trifluoromethyl)phenoxy)propyl)thio)phenoxy)acetic acid, or a pharmaceutically acceptable salt thereof.

The term “pharmaceutically acceptable salt” in reference to a PPARδ agonist refers to a salt of the PPARδ agonist, which does not cause significant irritation to a mammal to which it is administered and does not substantially abrogate the biological activity and properties of the compound. Handbook of Pharmaceutical Salts: Properties, Selection and Use. International Union of Pure and Applied Chemistry, Wiley-VCH 2002. S. M. Berge, L. D. Bighley, D. C. Monkhouse, J. Pharm. Sci. 1977, 66, 1-19. P. H. Stahl and C. G. Wermuth, editors, Handbook of Pharmaceutical Salts: Properties, Selection and Use, Weinheim/Zürich: Wiley-VCH/VHCA, 2002. In some embodiments, pharmaceutical salts typically are more soluble and more rapidly soluble in stomach and intestinal juices than non-ionic species and so are useful in solid dosage forms. Furthermore, because their solubility often is a function of pH, selective dissolution in one or another part of the digestive tract is possible and this capability, in some cases, is manipulated as one aspect of delayed and sustained release behaviors. Also, because the salt-forming molecule, in some cases, is in equilibrium with a neutral form, passage through biological membranes, in some cases, is adjusted.

In some embodiments, pharmaceutically acceptable salts are generally prepared by reacting the free base with a suitable organic or inorganic acid or by reacting the acid with a suitable organic or inorganic base. The term, in some embodiments, is used in reference to any compound of the present invention. Representative salts include the following salts: acetate, benzenesulfonate, benzoate, bicarbonate, bisulfate, bitartrate, borate, bromide, calcium edetate, camsylate, carbonate, chloride, clavulanate, citrate, dihydrochloride, edetate, edisylate, estolate, esylate, fumarate, gluceptate, gluconate, glutamate, glycollylarsanilate, hexylresorcinate, hydrabamine, hydrobromide, hydrochloride, hydroxynaphthoate, iodide, isethionate, lactate, lactobionate, laurate, malate, maleate, mandelate, mesylate, methylbromide, methylnitrate, methylsulfate, monopotassium maleate, mucate, napsylate, nitrate, n-methylglucamine, oxalate, pamoate (embonate), palmitate, pantothenate, phosphate/diphosphate, polygalacturonate, potassium, salicylate, sodium, stearate, subacetate, succinate, tannate, tartrate, teoclate, tosylate, triethiodide, trimethylammonium, and valerate. When an acidic substituent is present, such as —CO₂H, in some cases, formation of ammonium, morpholinium, sodium, potassium, barium, calcium salt, and the like for use as the dosage form. When a basic group is present, such as amino, or a basic heteroaryl radical, such as pyridyl, in some cases, formation of an acidic salt, such as hydrochloride, hydrobromide, phosphate, sulfate, trifluoroacetate, trichloroacetate, acetate, oxalate, maleate, pyruvate, malonate, succinate, citrate, tartarate, fumarate, mandelate, benzoate, cinnamate, methanesulfonate, ethanesulfonate, picrate, and the like, and include acids related to the pharmaceutically acceptable salts listed in Berge, et al., Journal of Pharmaceutical Sciences, Vol. 66(1), pp. 1-19 (1977).

### Certain Terminology

Unless otherwise stated, the following terms used in this application have the definitions given below. The use of the term “including” as well as other forms, such as “include”, “includes,” and “included,” is not limiting. The section headings used herein are for organizational purposes only and are not to be construed as limiting the subject matter described.

The term “acceptable” with respect to a formulation, composition or ingredient, as used herein, means having no persistent detrimental effect on the general health of the subject being treated.

The term “modulate” as used herein, means to interact with a target either directly or indirectly so as to alter the activity of the target, including, by way of example only, to enhance the activity of the target, to inhibit the activity of the target, to limit the activity of the target, or to extend the activity of the target.

The term “modulator” as used herein, refers to a molecule that interacts with a target either directly or indirectly. The interactions include, but are not limited to, the interactions of an agonist, partial agonist, an inverse agonist, antagonist, degrader, or combinations thereof. In some embodiments, a modulator is an antagonist. In some embodiments, a modulator is a degrader.

The terms “administer,” “administering”, “administration,” and the like, as used herein, refer to the methods that in some cases enable delivery of compounds or compositions to the desired site of biological action. These methods include, but are not limited to oral routes, intraduodenal routes, parenteral injection (including intravenous, subcutaneous, intraperitoneal, intramuscular, intravascular or infusion), topical and rectal administration. Those of skill in the art are familiar with administration techniques that can be employed with the compounds and methods described herein. In some embodiments, the compounds and compositions described herein are administered orally.

The terms “co-administration” or the like, as used herein, are meant to encompass administration of the selected therapeutic agents to a single patient and are intended to include treatment regimens in which the agents are administered by the same or different route of administration or at the same or different time.

The terms “effective amount” or “therapeutically effective amount,” as used herein, refer to a sufficient amount of an agent or a compound being administered, which will relieve to some extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is optionally determined using techniques, such as a dose escalation study.

The terms “enhance” or “enhancing,” as used herein, means to increase or prolong either in potency or duration a desired effect. Thus, in regard to enhancing the effect of therapeutic agents, the term “enhancing” refers to the ability to increase or prolong, either in potency or duration, the effect of other therapeutic agents on a system. An “enhancing-effective amount,” as used herein, refers to an amount adequate to enhance the effect of another therapeutic agent in a desired system.

The term “pharmaceutical combination” as used herein, means a product that results from the mixing or combining of more than one active ingredient and includes both fixed and non-fixed combinations of the active ingredients. The term “fixed combination” means that the active ingredients, e.g. a compound described herein, or a pharmaceutically acceptable salt thereof, and a co-agent, are both administered to a patient simultaneously in the form of a single entity or dosage. The term “non-fixed combination” means that the active ingredients, e.g. a compound described herein, or a pharmaceutically acceptable salt thereof, and a co-agent, are administered to a patient as separate entities either simultaneously, concurrently or sequentially with no specific intervening time limits, wherein such administration provides effective levels of the two compounds in the body of the patient. The latter also applies to cocktail therapy, e.g. the administration of three or more active ingredients.

The terms “kit” and “article of manufacture” are used as synonyms.

The term “subject” or “patient” encompasses mammals. Examples of mammals include, but are not limited to, any member of the Mammalian class: humans, non-human primates such as chimpanzees, and other apes and monkey species; farm animals such as cattle, horses, sheep, goats, swine; domestic animals such as rabbits, dogs, and cats; laboratory animals including rodents, such as rats, mice and guinea pigs, and the like. In one aspect, the mammal is a human.

The terms “treat,” “treating” or “treatment,” as used herein, include alleviating, abating or ameliorating at least one symptom of a disease or condition, preventing additional symptoms, inhibiting the disease or condition, e.g., arresting the development of the disease or condition, relieving the disease or condition, causing regression of the disease or condition, relieving a condition caused by the disease or condition, or stopping the symptoms of the disease or condition either prophylactically and/or therapeutically.

### Pharmaceutical Compositions

In some embodiments, the compounds described herein are formulated into pharmaceutical compositions. Pharmaceutical compositions are formulated in a conventional manner using one or more pharmaceutically acceptable inactive ingredients that facilitate processing of the active compounds into preparations that are used pharmaceutically. Proper formulation is dependent upon the route of administration chosen. A summary of pharmaceutical compositions described herein is found, for example, in Remington: The Science and Practice of Pharmacy, Nineteenth Ed (Easton, Pa.: Mack Publishing Company, 1995); Hoover, John E., Remington's Pharmaceutical Sciences, Mack Publishing Co., Easton, Pa. 1975; Liberman, H. A. and Lachman, L., Eds., Pharmaceutical Dosage Forms, Marcel Decker, New York, N.Y., 1980; and Pharmaceutical Dosage Forms and Drug Delivery Systems, Seventh Ed. (Lippincott Williams & Wilkins 1999), herein incorporated by reference for such disclosure.

In some embodiments, the compounds described herein are administered either alone or in combination with pharmaceutically acceptable carriers, excipients or diluents, in a pharmaceutical composition. Administration of the compounds and compositions described herein, in some cases, are effected by any method that enables delivery of the compounds to the site of action. These methods include, though are not limited to delivery via enteral routes (including oral, gastric or duodenal feeding tube, rectal suppository and rectal enema), parenteral routes (injection or infusion, including intraarterial, intracardiac, intradermal, intraduodenal, intramedullary, intramuscular, intraosseous, intraperitoneal, intrathecal, intravascular, intravenous, intravitreal, epidural and subcutaneous), inhalational, transdermal, transmucosal, sublingual, buccal and topical (including epicutaneous, dermal, enema, eye drops, ear drops, intranasal, vaginal) administration, although the most suitable route, in some instances, depends upon for example the condition and disorder of the recipient. By way of example only, compounds described herein, in some cases, are administered locally to the area in need of treatment, by for example, local infusion during surgery, topical application such as creams or ointments, injection, catheter, or implant. The administration, in some cases, is by direct injection at the site of a diseased tissue or organ.

In some embodiments of the invention, a PPARδ agonist is included within a pharmaceutical composition. As used herein, the term “pharmaceutical composition” refers to a liquid or solid composition, preferably solid (e.g., a granulated powder), that contains a pharmaceutically active ingredient (e.g., a PPARδ agonist) and at least a carrier, where none of the ingredients is generally biologically undesirable at the administered quantities.

Pharmaceutical compositions incorporating a PPARδ agonist, in some cases, take any physical form that is pharmaceutically acceptable. Pharmaceutical compositions for oral administration are particularly preferred. In one embodiment of such pharmaceutical compositions, an effective amount of a PPARδ agonist is incorporated.

In some cases, known methods of formulating pharmaceutical compositions that are typically used in the pharmaceutical sciences are followed. All of the usual types of compositions are contemplated, including, but not limited to, tablets, chewable tablets, capsules, and solutions. The amount of the PPARδ agonist, however, is best defined as the effective amount, that is, the amount of the PPARδ agonist that provides the desired dose to the subject in need of such treatment. Any of the PPARδ agonists as described herein are formulated in any desired form of composition.

Capsules, in some cases, are prepared by mixing the PPARδ agonist with a suitable diluent and filling the proper amount of the mixture in capsules. The usual diluents include inert powdered substances such as starch of many different kinds, powdered cellulose, especially crystalline and microcrystalline cellulose, sugars such as fructose, mannitol and sucrose, grain flours and similar edible powders.

Tablets, in some cases, are prepared by direct compression, by wet granulation, or by dry granulation. Their formulations usually incorporate diluents, binders, lubricants, and disintegrators, as well as the PPARδ agonist. Typical diluents include, for example, various types of starch, lactose, mannitol, kaolin, calcium phosphate or sulfate, inorganic salts such as sodium chloride, and powdered sugar. Powdered cellulose derivatives are also useful. Typical tablet binders are substances such as starch, gelatin, and sugars such as lactose, fructose, glucose, and the like. Natural and synthetic gums are also convenient, including acacia, alginates, methylcellulose, polyvinylpyrrolidine, and the like. Polyethylene glycol, ethylcellulose, and waxes, in some cases, also serve as binders.

A lubricant in a tablet formulation, in some cases, help prevent the tablet and punches from sticking in the die. A lubricant, in some cases, is chosen from such solids as talc, magnesium and calcium stearate, stearic acid, and hydrogenated vegetable oils.

Tablet disintegrators are substances that swell when wetted to break up the tablet and release the compound. They include starches, clays, celluloses, aligns, and gums. More particularly, corn and potato starches, methylcellulose, agar, bentonite, wood cellulose, powdered natural sponge, cation-exchange resins, alginic acid, guar gum, citrus pulp, and carboxymethylcellulose, for example, in some cases, are used, as well as sodium lauryl sulfate.

Enteric formulations are often used to protect an active ingredient from the strongly acidic contents of the stomach. Such formulations are created by coating a solid dosage form with a film of a polymer that is insoluble in acid environments, and soluble in basic environments. Exemplary films are cellulose acetate phthalate, polyvinyl acetate phthalate, hydroxypropyl methylcellulose phthalate, and hydroxypropyl methylcellulose acetate succinate.

Tablets are often coated with sugar as a flavor and sealant. The PPARδ agonists, in some cases, are also be formulated as chewable tablets by using large amounts of pleasant-tasting substances, such as mannitol, in the formulation.

Transdermal patches, in some cases, are used. Typically, a patch comprises a resinous composition in which the active compound(s) will dissolve, or partially dissolve, and is held in contact with the skin by a film that protects the composition. Other, more complicated patch compositions are also in use, particularly those having a membrane pierced with innumerable pores through which the drugs are pumped by osmotic action.

In any embodiment where a PPARδ agonist is included in a pharmaceutical composition, such pharmaceutical compositions, in some cases, are in a form suitable for oral use, for example, as tablets, troches, lozenges, aqueous or oily suspensions, dispersible powders or granules, emulsions, hard or soft capsules, or syrups or elixirs. Compositions intended for oral use, in some cases, are prepared according to any known method, and such compositions, in some cases, contain one or more agents selected from the group consisting of sweetening agents, flavoring agents, coloring agents, and preserving agents in order to provide pharmaceutically elegant and palatable preparations. Tablets, in some cases, contain the active ingredient in admixture with non-toxic pharmaceutically acceptable excipients that are suitable for the manufacture of tablets. These excipients include for example, inert diluents, such as calcium carbonate, sodium carbonate, lactose, calcium phosphate, or sodium phosphate; granulating and disintegrating agents, for example, corn starch or alginic acid; binding agents, for example, starch, gelatin, or acacia; and lubricating agents, for example, magnesium stearate, stearic acid, or talc. The tablets, in some cases, are uncoated or they, in some cases, are coated by known techniques to delay disintegration and absorption in the gastrointestinal tract and thereby provide a sustained action over a longer period. For example, a time delay material such as glyceryl monostearate or glyceryl distearate, in some cases, is employed.

### Methods of Dosing and Treatment Regimens

In one embodiment, a PPARδ agonist (e.g. Compound 1, or a pharmaceutically acceptable salt thereof), is used in the preparation of medicaments for the treatment of fatty acid oxidation disorders (FAOD) in a mammal. Methods for treating any of the diseases or conditions described herein in a mammal in need of such treatment, involves administration of pharmaceutical compositions that include a PPARδ agonist (e.g. Compound 1, or a pharmaceutically acceptable salt thereof), active metabolite, prodrug, in therapeutically effective amounts to said mammal.

In certain embodiments, the compositions containing the compound(s) described herein are administered for prophylactic and/or therapeutic treatments. In certain therapeutic applications, the compositions are administered to a patient already suffering from a disease or condition, in an amount sufficient to cure or at least partially arrest at least one of the symptoms of the disease or condition. Amounts effective for this use depend on the severity and course of the disease or condition, previous therapy, the patient's health status, weight, and response to the drugs, and the judgment of the treating physician. Therapeutically effective amounts are optionally determined by methods including, but not limited to, a dose escalation and/or dose ranging clinical trial.

In prophylactic applications, compositions containing a PPARδ agonist (e.g. Compound 1, or a pharmaceutically acceptable salt thereof), are administered to a patient susceptible to or otherwise at risk of a particular disease, disorder or condition. Such an amount is defined to be a “prophylactically effective amount or dose.” In this use, the precise amounts also depend on the patient's state of health, weight, and the like. When used in patients, effective amounts for this use will depend on the severity and course of the disease, disorder or condition, previous therapy, the patient's health status and response to the drugs, and the judgment of the treating physician. In one aspect, prophylactic treatments include administering to a mammal, who previously experienced at least one symptom of the disease being treated and is currently in remission, a pharmaceutical composition comprising a PPARδ agonist (e.g. Compound 1, or a pharmaceutically acceptable salt thereof), in order to prevent a return of the symptoms of the disease or condition.

In certain embodiments wherein the patient's condition does not improve, upon the doctor's discretion the administration of a PPARδ agonist (e.g. Compound 1, or a pharmaceutically acceptable salt thereof), is administered chronically, that is, for an extended period of time, including throughout the duration of the patient's life in order to ameliorate or otherwise control or limit the symptoms of the patient's disease or condition.

In certain embodiments wherein a patient's status does improve, the dose of drug being administered is temporarily reduced or temporarily suspended for a certain length of time (i.e., a “drug holiday”). In specific embodiments, the length of the drug holiday is between 2 days and 1 year, including by way of example only, 2 days, 3 days, 4 days, 5 days, 6 days, 7 days, 10 days, 12 days, 15 days, 20 days, 28 days, or more than 28 days. The dose reduction during a drug holiday is, by way of example only, by about 10%-100%, including by way of example only about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, and about 100%.

Once improvement of the patient's conditions has occurred, a maintenance dose is administered if necessary. Subsequently, in specific embodiments, the dosage or the frequency of administration, or both, is reduced, as a function of the symptoms, to a level at which the improved disease, disorder or condition is retained. In certain embodiments, however, the patient requires intermittent treatment on a long-term basis upon any recurrence of symptoms.

In one aspect, a PPARδ agonist (e.g. Compound 1, or a pharmaceutically acceptable salt thereof), is administered daily to humans with a FAOD in need of therapy with a PPARδ agonist (e.g. Compound 1, or a pharmaceutically acceptable salt thereof). In some embodiments, a PPARδ agonist (e.g. Compound 1, or a pharmaceutically acceptable salt thereof), is administered once-a-day. In some embodiments, a PPARδ agonist (e.g. Compound 1, or a pharmaceutically acceptable salt thereof), is administered twice-a-day. In some embodiments, a PPARδ agonist (e.g. Compound 1, or a pharmaceutically acceptable salt thereof), is administered three times-a-day. In some embodiments, a PPARδ agonist (e.g. Compound 1, or a pharmaceutically acceptable salt thereof), is administered every other day. In some embodiments, a PPARδ (e.g. Compound 1, or a pharmaceutically acceptable salt thereof), is administered twice a week.

In some instances, a PPARδ agonist (e.g. Compound 1, or a pharmaceutically acceptable salt thereof) is administered once per day, twice per day, three times per day or more. In some instances, a PPARδ agonist (e.g. Compound 1, or a pharmaceutically acceptable salt thereof) is administered twice per day. A PPARδ agonist (e.g. Compound 1, or a pharmaceutically acceptable salt thereof), in some embodiments, is administered daily, every day, every alternate day, five days a week, once a week, every other week, two weeks per month, three weeks per month, once a month, twice a month, three times per month, or more. In some embodiments, a PPARδ agonist (e.g. Compound 1, or a pharmaceutically acceptable salt thereof) is administered twice daily, e.g., morning and evening. In some embodiments, a PPARδ agonist (e.g. Compound 1, or a pharmaceutically acceptable salt thereof) is administered for at least 1 day, 2 days, 3 days, 4 days, 5 days, 6 days, 1 week, 2 weeks, 3 weeks, 1 month, 2 months, 3 months, 4 months, 5 months, 6 months, 7 months, 8 months, 9 months, 10 months, 11 months, 12 months, 18 months, 2 years, 3 years, 4 years, 5 years, 10 years, or more. In some embodiments, a PPARδ agonist (e.g. Compound 1, or a pharmaceutically acceptable salt thereof) is administered twice daily for at least or about 1 week, 2 weeks, 3 weeks, 1 month, 2 months, 3 months, 4 months, 5 months, 6 months, or more. In some embodiments, a PPARδ agonist (e.g. Compound 1, or a pharmaceutically acceptable salt thereof) is administered once daily, twice daily, three times daily, four times daily, or more than four times daily for at least or about 1 week, 2 weeks, 3 weeks, 1 month, 2 months, 3 months, 4 months, 5 months, 6 months, or more.

In general, doses of a PPARδ agonist (e.g. Compound 1, or a pharmaceutically acceptable salt thereof), employed for treatment of the diseases or conditions described herein in humans are typically in the range of from about 0.1 mg/kg to about 10 mg/kg of body weight per dose. In one embodiment, the desired dose is conveniently presented in a single dose or in divided doses administered simultaneously (or over a short period of time) or at appropriate intervals, for example as two, three, four or more sub-doses per day. In some embodiments, a PPARδ agonist (e.g. Compound 1, or a pharmaceutically acceptable salt thereof), is conveniently presented in divided doses that are administered simultaneously (or over a short period of time) once a day. In some embodiments, a PPARδ agonist (e.g. Compound 1, or a pharmaceutically acceptable salt thereof), is conveniently presented in divided doses that are administered in equal portions twice-a-day.

In some embodiments, a PPARδ agonist (e.g. Compound 1, or a pharmaceutically acceptable salt thereof), is administered orally to the human at a dose from about 0.1 mg to about 10 mg/kg of body weight per dose. In some embodiments, a PPARδ agonist (e.g. Compound 1, or a pharmaceutically acceptable salt thereof), is administered to the human on a continuous dosing schedule. In some embodiments, a PPARδ agonist (e.g. Compound 1, or a pharmaceutically acceptable salt thereof), is administered to the human on a continuous daily dosing schedule.

The term “continuous dosing schedule” refers to the administration of a particular therapeutic agent at regular intervals. In some embodiments, continuous dosing schedule refers to the administration of a particular therapeutic agent at regular intervals without any drug holidays from the particular therapeutic agent. In some other embodiments, continuous dosing schedule refers to the administration of a particular therapeutic agent in cycles. In some other embodiments, continuous dosing schedule refers to the administration of a particular therapeutic agent in cycles of drug administration followed by a drug holiday (for example, a wash out period or other such period of time when the drug is not administered) from the particular therapeutic agent. For example, in some embodiments the therapeutic agent is administered once a day, twice a day, three times a day, once a week, twice a week, three times a week, four times a week, five times a week, six times a week, seven times a week, every other day, every third day, every fourth day, daily for a week followed by a week of no administration of the therapeutic agent, daily for a two weeks followed by one or two weeks of no administration of the therapeutic agent, daily for three weeks followed by one, two or three weeks of no administration of the therapeutic agent, daily for four weeks followed by one, two, three or four weeks of no administration of the therapeutic agent, weekly administration of the therapeutic agent followed by a week of no administration of the therapeutic agent, or biweekly administration of the therapeutic agent followed by two weeks of no administration of the therapeutic agent. In some embodiments, daily administration is once a day. In some embodiments, daily administration is twice a day. In some embodiments, daily administration is three times a day. In some embodiments, daily administration is more than three times a day.

The term “continuous daily dosing schedule” refers to the administration of a particular therapeutic agent every day at roughly the same time each day. In some embodiments, daily administration is once a day. In some embodiments, daily administration is twice a day. In some embodiments, daily administration is three times a day. In some embodiments, daily administration is more than three times a day.

In some embodiments, the amount of a PPARδ agonist (e.g. Compound 1, or a pharmaceutically acceptable salt thereof), is administered once a day. In some other embodiments, the amount of a PPARδ agonist (e.g. Compound 1, or a pharmaceutically acceptable salt thereof), is administered twice a day. In some other embodiments, the amount of a PPARδ agonist (e.g. Compound 1, or a pharmaceutically acceptable salt thereof), is administered three times a day.

In certain embodiments wherein improvement in the status of the disease or condition in the human is not observed, the daily dose of a PPARδ agonist (e.g. Compound 1, or a pharmaceutically acceptable salt thereof), is increased. In some embodiments, a once-a-day dosing schedule is changed to a twice-a-day dosing schedule. In some embodiments, a three times a day dosing schedule is employed to increase the amount of a PPARδ agonist (e.g. Compound 1, or a pharmaceutically acceptable salt thereof), that is administered. In some embodiments, the frequency of administration by inhalation is increased in order to provide repeat high Cmax levels on a more regular basis. In some embodiments, the frequency of administration is increased in order to provide maintained or more regular exposure to a PPARδ agonist (e.g. Compound 1, or a pharmaceutically acceptable salt thereof). In some embodiments, the frequency of administration is increased in order to provide repeat high Cmax levels on a more regular basis and provide maintained or more regular exposure to a PPARδ agonist (e.g. Compound 1, or a pharmaceutically acceptable salt thereof).

In any of the aforementioned aspects are further embodiments comprising single administrations of the effective amount of a PPARδ agonist (e.g. Compound 1, or a pharmaceutically acceptable salt thereof), including further embodiments in which the PPARδ agonist, is administered (i) once a day; or (ii) multiple times over the span of one day.

In any of the aforementioned aspects are further embodiments comprising multiple administrations of the effective amount of a PPARδ agonist (e.g. Compound 1, or a pharmaceutically acceptable salt thereof), including further embodiments in which (i) the PPARδ agonist is administered continuously or intermittently: as in a single dose; (ii) the time between multiple administrations is every 6 hours; (iii) the PPARδ agonist is administered to the mammal every 8 hours; (iv) the PPARδ agonist is administered to the mammal every 12 hours; (v) the PPARδ agonist is administered to the mammal every 24 hours. In further or alternative embodiments, the method comprises a drug holiday, wherein the administration of the PPARδ agonist is temporarily suspended or the dose of the PPARδ agonist being administered is temporarily reduced; at the end of the drug holiday, dosing of the PPARδ agonist is resumed. In one embodiment, the length of the drug holiday varies from 2 days to 1 year.

Generally, a suitable dose of a PPARδ agonist, or a pharmaceutically acceptable salt thereof, for administration to a human will be in the range of about 0.1 mg/kg per day to about 25 mg/kg per day (e.g., about 0.2 mg/kg per day, about 0.3 mg/kg per day, about 0.4 mg/kg per day, about 0.5 mg/kg per day, about 0.6 mg/kg per day, about 0.7 mg/kg per day, about 0.8 mg/kg per day, about 0.9 mg/kg per day, about 1 mg/kg per day, about 2 mg/kg per day, about 3 mg/kg per day, about 4 mg/kg per day, about 5 mg/kg per day, about 6 mg/kg per day, about 7 mg/kg per day, about 8 mg/kg per day, about 9 mg/kg per day, about 10 mg/kg per day, about 15 mg/kg per day, about 20 mg/kg per day, or about 25 mg/kg per day). Alternatively, a suitable dose of a PPARδ agonist, or a pharmaceutically acceptable salt thereof, for administration to a human will be in the range of from about 0.1 mg/day to about 1000 mg/day; from about 1 mg/day to about 400 mg/day; or from about 1 mg/day to about 300 mg/day. In other embodiments, a suitable dose of a PPARδ agonist, or a pharmaceutically acceptable salt thereof, for administration to a human will be about 1 mg/day, about 2 mg/day, about 3 mg/day, about 4 mg/day, about 5 mg/day, about 6 mg/day, about 7 mg/day, about 8 mg/day, about 9 mg/day, about 10 mg/day, about 15 mg/day, about 20 mg/day, about 25 mg/day, about 30 mg/day, about 35 mg/day, about 40 mg/day, about 45 mg/day, about 50 mg/day, about 55 mg/day, about 60 mg/day, about 65 mg/day, about 70 mg/day, about 75 mg/day, about 80 mg/day, about 85 mg/day, about 90 mg/day, about 95 mg/day, about 100 mg/day, about 125 mg/day, about 150 mg/day, about 175 mg/day, about 200 mg/day, about 225 mg/day, about 250 mg/day, about 275 mg/day, about 300 mg/day, about 325 mg/day, about 350 mg/day, about 375 mg/day, about 400 mg/day, about 425 mg/day, about 450 mg/day, about 475 mg/day, or about 500 mg/day. Dosages, in some cases, are administered more than one time per day (e.g., two, three, four, or more times per day). In one embodiment, a suitable dose of a PPARδ agonist, or a pharmaceutically acceptable salt thereof, for administration to a human is about 100 mg twice/day (i.e., a total of about 200 mg/day). In another embodiment, a suitable dose of a PPARδ agonist, or a pharmaceutically acceptable salt thereof, for administration to a human is about 50 mg twice/day (i.e., a total of about 100 mg/day).

In some embodiments, the daily dosage or the amount of active in the dosage form are lower or higher than the ranges indicated herein, based on a number of variables in regard to an individual treatment regime. In various embodiments, the daily and unit dosages are altered depending on a number of variables including, but not limited to, the disease or condition to be treated, the mode of administration, the requirements of the individual subject, the severity of the disease or condition being treated, the identity (e.g., weight) of the human, and the particular additional therapeutic agents that are administered (if applicable), and the judgment of the practitioner.

Toxicity and therapeutic efficacy of such therapeutic regimens are determined by standard pharmaceutical procedures in cell cultures or experimental animals, including, but not limited to, the determination of the LD₅₀ and the ED₅₀. The dose ratio between the toxic and therapeutic effects is the therapeutic index and it is expressed as the ratio between LD₅₀ and ED₅₀. In certain embodiments, the data obtained from cell culture assays and animal studies are used in formulating the therapeutically effective daily dosage range and/or the therapeutically effective unit dosage amount for use in mammals, including humans. In some embodiments, the daily dosage amount of the PPARδ agonist lies within a range of circulating concentrations that include the ED₅₀ with minimal toxicity. In certain embodiments, the daily dosage range and/or the unit dosage amount varies within this range depending upon the dosage form employed and the route of administration utilized.

In some embodiments, following the administration of a therapeutically effective dose of the PPARδ agonist to a subject, the no observed adverse effect level (NOAEL) is at least 1, 10, 20, 50, 100, 500 or 1000 milligrams of the PPARδ agonist per kilogram of body weight (mpk). In some examples, the 7-day NOAEL for a rat administered PPARδ agonist is at least about 200, 300, 400, 500, 600, 700, 800, 900, 1000, 1500 or 2000 mpk. In some examples, the 7-day NOAEL for a dog administered PPARδ agonist is at least about 10, 20, 30, 40, 50, 60, 70, 80, 90, 100, 200, 500 mpk.

In some embodiments, methods for treating a fatty acid oxidation disorder (FAOD) in a mammal with a PPARδ agonist compound described herein (e.g. Compound 1, or a pharmaceutically acceptable salt thereof) results in improvements in one or more outcome measures. In some embodiments, outcomes measures include, but are not limited to: patient reported outcomes (PRO), exercise tolerance, whole body fatty acid oxidation (e.g. ¹³CO₂ production), blood acylcarnitines profiles, and blood inflammatory cytokines. In some embodiments, a baseline assessment is determined, typically prior to the administration of a PPARδ agonist (e.g. Compound 1, or a pharmaceutically acceptable salt thereof). Improvements in outcome measures are assessed with repeated assessments taken during treatment with a PPARδ agonist compound and a comparison against the baseline assessment and/or any prior assessment(s). In some embodiments, improvements are by at least or about 10%, 15%, 20%, 25%, 30%, 35%, 40%, 45%, 50%, 55%, 60%, 65%, 70%, 75%, 80%, 85%, 90%, 95%, or more than 95%. In some embodiments, the PPARδ agonist (e.g. Compound 1, or a pharmaceutically acceptable salt thereof) described herein improvements are by at least or about 0.5×, 1.0×, 1.5×, 2.0×, 2.5×, 3.0×, 3.5×, 4.0×, 5.0×, 6.0×, 7.0×, 8.0×, 9.0×, 10×, or more than 10×. Improvements, in some embodiments, are compared to a control. In some embodiments, a control is an individual who does not receive a PPARδ agonist (e.g. Compound 1, or a pharmaceutically acceptable salt thereof). In some embodiments, the control is an individual who does not receive a full dose of a PPARδ agonist (e.g. Compound 1, or a pharmaceutically acceptable salt thereof). In some embodiments, the control is baseline for the individual prior to receiving a PPARδ agonist (e.g. Compound 1, or a pharmaceutically acceptable salt thereof).

In some embodiments, patient reported outcomes (PRO) are measured with questionnaires. In some embodiments, the questionnaire covers health concepts related to the disorder being treated. In some embodiments, the questionnaire covers health concepts related to the disorder being treated such as, but not, limited to: physical functioning, bodily pain, role limitations due to physical health problems, role limitations due to personal or emotional problems, emotional well-being, social functioning, energy/fatigue, and general health perceptions, including perceptions in change of health.

In some embodiments, outcome measures are assessed with tests that assess exercise tolerance. In some embodiments, exercise tolerance is assessed with exercise tests. Exercise tests include, but are not limited to, submaximal treadmill, walking tests (e.g. without limitation, 6 minute; 12 minute walks), run tests, treadmill and ergometry exercise testing. In some embodiments, exercise tests are used in combination with the Borg Scale of perceived exertion. In some embodiments, exercise tests are performed according to guidelines set forth by the American Thoracic Society (ATS).

In some embodiments, the respiratory exchange ratio (RER) is measured to assess exercise tolerance. RER is the ratio between the amount of carbon dioxide (CO₂) produced in metabolism and oxygen (O₂) used. In some embodiments, the ratio is determined by comparing exhaled gases to room air.

PPAR agonists have demonstrated the ability to increase ¹³CO₂ production in clinical trials (Gillingham, M. B., et al., Journal of Inherited Metabolic Disease, Volume 40, Issue 6, November 2017, 831-843; Riserus, U., et al. Diabetes 2008 February; 57(2): 332-339; each of which is incorporated for such protocols). In some embodiments, stable isotope methods are used to measure in vivo residual fatty acid oxidation capacity. Enrichment of ¹³CO₂ only occurs by one complete round of fatty acid oxidation. A representative protocol is as follows. A fasting blood sample is obtained after an overnight fast. Prior to breakfast, a resting indirect calorimetry is measured. Subjects are then given a meal (e.g a shake) containing 17-mg/kg ¹³C-oleic acid. Breath samples are collected prior to (time 0) and again hourly at 1, 2, 3, 4, 5, 6, 7, and 8 hours following the ¹³C-oleic administration. ¹³C in breath samples are measured as a ratio of ¹³C/¹²C using the Delta Plus IRMS (Finnigan MAT, Bremen, Germany). Recovery is calculated as ¹³C divided by the dose of ¹³C administered. The amount of excess ¹³C in breath is a measure of residual fatty acid oxidation capacity in subjects with disorders of long-chain fatty acid oxidation.

In some embodiments, improvements in fatty acid oxidation in subjects with a FAOD that are treated with a PPARδ agonist compound described herein (e.g. Compound 1, or a pharmaceutically acceptable salt thereof) are measured with a suitable ¹³CO₂ breath sample test. In some embodiments, a suitable ¹³CO₂ breath sample test comprises the steps of: 1) providing the subject a meal comprising ¹³C-enriched fatty acid(s); 2) administering to the subject a PPARδ agonist compound, or a pharmaceutically acceptable salt thereof, after the consumption of the meal; and 3) collecting breath samples from the subject at regular intervals and measuring the relative amount of ¹³CO2 to ¹²CO₂ in the breath samples. In some embodiments, the breath samples are collected about every hour. In some embodiments, the meal is enriched with a 13C labeled fatty acid, wherein the fatty acid is butyric acid, caproic acid, caprylic acid, capric acid, lauric acid, myristic acid, palmitic acid, stearic acid, arachidic acid, behenic acid, lignoceric acid, caproleic acid, lauroleic acid, myristoleic acid, palmitoleic acid, oleic acid, elaidic acid, vaccenic acid, gadoleic acid, erucic acid, brassidic acid, nervonic acid, linoleic acid, alpha-linolenic acid, gamma-linolenic acid, columbinic acid, stearidonic acid, mead acid, dihomo-γ-linolenic acid, arachidonic acid, eicosapentaenoic acid, docosapentaenoic acid, or docosahexaenoic acid.

In some embodiments, described herein is a method for measuring whole-body fatty acid oxidation in a human with a fatty acid oxidation disorder (FAOD) comprising: feeding the human with a fatty acid oxidation disorder (FAOD) a meal comprising ¹³C-enriched fatty acids and measuring the amount of exhaled ¹³CO₂ from the human, wherein the human with a fatty acid oxidation disorder (FAOD) is undergoing treatment with a PPARδ agonist compound.

In some embodiments, described herein is a method for measuring changes in whole-body fatty acid oxidation in a human with a fatty acid oxidation disorder (FAOD) comprising the steps of: 1) providing a meal enriched with a ¹³C labeled fatty acid; 2) administering to the human a PPARδ agonist compound, or a pharmaceutically acceptable salt thereof, and 3) collecting breath samples from the human at regular intervals and measuring for the content of ¹³CO₂ in the breath samples.

In some embodiments, the amount of ¹³CO₂ in breath samples is used as a diagnostic to guide treatment of the subject with a FAOD with a PPARδ agonist compound. For example, if a subject or individual has a change in the amount of ¹³CO₂ of at least a specified percentage or level following the administration of a PPARδ agonist compound, the subject or individual continues the treatment using a PPARδ agonist (e.g. Compound 1, or a pharmaceutically acceptable salt thereof) described herein. In some embodiments, modest increases in ¹³CO₂ in breath samples may necessitate an increase in the amount of PPARδ agonist compound that is administered to the subject, an increase in the frequency of administering the PPARδ agonist compound, or both.

In some instances, the change in the amount of ¹³CO₂ is at least or about 10%, 15%, 20%, 25%, 30%, 35%, 40%, 45%, 50%, 55%, 60%, 65%, 70%, 75%, 80%, 85%, 90%, 95%, or more than 95% compared to baseline. In some instances, the change occurs after at least or about 1 hour, 2 hours, 3 hours, 4 hours, 5 hours, 6 hours, 7 hours, 8 hours, 12 hours, 16 hours, 20 hours, 1 day, 2 days, 3 days, 4 days, 5 days, 6 days, 1 week, 2 weeks, 3 weeks, 1 month, 2 months, 3 months, 4 months, or more than 4 months after initiation of treatment with a PPARδ agonist compound (e.g. Compound 1, or a pharmaceutically acceptable salt thereof) has begun. In some instances, a treatment regimen comprising a PPARδ agonist compound (e.g. Compound 1, or a pharmaceutically acceptable salt thereof) is continued if the change in the amount of ¹³CO₂ is at least or about 10%, 15%, 20%, 25%, 30%, 35%, 40%, 45%, 50%, 55%, 60%, 65%, 70%, 75%, 80%, 85%, 90%, 95%, or more than 95% compared to baseline after at least or about 1 hour, 2 hours, 3 hours, 4 hours, 5 hours, 6 hours, 7 hours, 8 hours, 12 hours, 16 hours, 20 hours, 1 day, 2 days, 3 days, 4 days, 5 days, 6 days, 1 week, 2 weeks, 3 weeks, 1 month, 2 months, 3 months, 4 months, or more than 4 months after initiation of treatment with a PPARδ agonist compound (e.g. Compound 1, or a pharmaceutically acceptable salt thereof) has begun. In some instances, the change is an increase in the levels of ¹³CO₂.

In some embodiments, increases of amount of ¹³CO₂ over time is indicative of a subject's responsive to the PPARδ agonist compound (e.g. Compound 1, or a pharmaceutically acceptable salt thereof). In some instances, a subject is responsive to the PPARδ agonist compound (e.g. Compound 1, or a pharmaceutically acceptable salt thereof) if there is a change in the amount of ¹³CO₂ of at least or about 10%, 15%, 20%, 25%, 30%, 35%, 40%, 45%, 50%, 55%, 60%, 65%, 70%, 75%, 80%, 85%, 90%, 95%, or more than 95% compared to baseline in ¹³CO₂ levels. In some instances, the change in the amount of ¹³CO₂ occurs after at least or about 1 hour, 2 hours, 3 hours, 4 hours, 5 hours, 6 hours, 7 hours, 8 hours, 12 hours, 16 hours, 20 hours, 1 day, 2 days, 3 days, 4 days, 5 days, 6 days, 1 week, 2 weeks, 3 weeks, 1 month, 2 months, 3 months, 4 months, or more than 4 months after administration of the PPARδ agonist (e.g. Compound 1, or a pharmaceutically acceptable salt thereof) described herein. In some instances, a subject is responsive if the change in the amount of ¹³CO₂ is at least or about 10%, 15%, 20%, 25%, 30%, 35%, 40%, 45%, 50%, 55%, 60%, 65%, 70%, 75%, 80%, 85%, 90%, 95%, or more than 95% compared to baseline after at least or about 1 hour, 2 hours, 3 hours, 4 hours, 5 hours, 6 hours, 7 hours, 8 hours, 12 hours, 16 hours, 20 hours, 1 day, 2 days, 3 days, 4 days, 5 days, 6 days, 1 week, 2 weeks, 3 weeks, 1 month, 2 months, 3 months, 4 months, or more than 4 months after initiation of treatment with a PPARδ agonist compound (e.g. Compound 1, or a pharmaceutically acceptable salt thereof) has begun. In some instances, the change is an increase in the amount of ¹³CO₂ in the breath samples overt time.

### Combination Treatments

In certain instances, it is appropriate to administer a PPARδ agonist (e.g. Compound 1, or a pharmaceutically acceptable salt thereof), in combination with one or more other therapeutic agents.

In one embodiment, the therapeutic effectiveness of a PPARδ agonist (e.g. Compound 1), or a pharmaceutically acceptable salt or solvate thereof, is enhanced by administration of an adjuvant (i.e., by itself the adjuvant has minimal therapeutic benefit, but in combination with another therapeutic agent, the overall therapeutic benefit to the patient is enhanced). Or, in some embodiments, the benefit experienced by a patient is increased by administering a PPARδ agonist (e.g. Compound 1), or a pharmaceutically acceptable salt or solvate thereof, with another agent (which also includes a therapeutic regimen) that also has therapeutic benefit.

In one specific embodiment, a PPARδ agonist (e.g. Compound 1), or a pharmaceutically acceptable salt or solvate thereof, is co-administered with a second therapeutic agent, wherein a PPARδ agonist (e.g. Compound 1), or a pharmaceutically acceptable salt or solvate thereof, and the second therapeutic agent modulate different aspects of the disease, disorder or condition being treated, thereby providing a greater overall benefit than administration of either therapeutic agent alone.

In any case, regardless of the disease, disorder or condition being treated, the overall benefit experienced by the patient is simply additive of the two therapeutic agents or the patient experiences a synergistic benefit.

In certain embodiments, different therapeutically-effective dosages of a PPARδ agonist (e.g. Compound 1), or a pharmaceutically acceptable salt or solvate thereof, will be utilized in formulating pharmaceutical composition and/or in treatment regimens when a PPARδ agonist (e.g. Compound 1), or a pharmaceutically acceptable salt or solvate thereof, is administered in combination with one or more additional agent, such as an additional therapeutically effective drug, an adjuvant or the like. Therapeutically-effective dosages of drugs and other agents for use in combination treatment regimens is optionally determined by means similar to those set forth hereinabove for the actives themselves. Furthermore, the methods of prevention/treatment described herein encompasses the use of metronomic dosing, i.e., providing more frequent, lower doses in order to minimize toxic side effects. In some embodiments, a combination treatment regimen encompasses treatment regimens in which administration of a PPARδ agonist (e.g. Compound 1), or a pharmaceutically acceptable salt or solvate thereof, is initiated prior to, during, or after treatment with a second agent described herein, and continues until any time during treatment with the second agent or after termination of treatment with the second agent. It also includes treatments in which a PPARδ agonist (e.g. Compound 1), or a pharmaceutically acceptable salt or solvate thereof, and the second agent being used in combination are administered simultaneously or at different times and/or at decreasing or increasing intervals during the treatment period. Combination treatment further includes periodic treatments that start and stop at various times to assist with the clinical management of the patient.

It is understood that the dosage regimen to treat, prevent, or ameliorate the condition(s) for which relief is sought, is modified in accordance with a variety of factors (e.g. the disease, disorder or condition from which the subject suffers; the age, weight, sex, diet, and medical condition of the subject). Thus, in some instances, the dosage regimen actually employed varies and, in some embodiments, deviates from the dosage regimens set forth herein.

For combination therapies described herein, dosages of the co-administered compounds vary depending on the type of co-drug employed, on the specific drug employed, on the disease or condition being treated and so forth. In additional embodiments, when co-administered with one or more other therapeutic agents, a PPARδ agonist (e.g. Compound 1), or a pharmaceutically acceptable salt or solvate thereof, is administered either simultaneously with the one or more other therapeutic agents, or sequentially.

In combination therapies, the multiple therapeutic agents (one of which is a PPARδ agonist (e.g. Compound 1), or a pharmaceutically acceptable salt or solvate thereof) are administered in any order or even simultaneously. If administration is simultaneous, the multiple therapeutic agents are, by way of example only, provided in a single, unified form, or in multiple forms (e.g., as a single pill or as two separate pills).

A PPARδ agonist (e.g. Compound 1), or a pharmaceutically acceptable salt or solvate thereof, as well as combination therapies, are administered before, during or after the occurrence of a disease or condition, and the timing of administering the composition containing a PPARδ agonist (e.g. Compound 1), or a pharmaceutically acceptable salt or solvate thereof, varies. Thus, in one embodiment, Compound I, or a pharmaceutically acceptable salt or solvate thereof, is used as a prophylactic and are administered continuously to subjects with a propensity to develop conditions or diseases in order to prevent the occurrence of the disease or condition. In another embodiment, a PPARδ agonist (e.g. Compound 1), or a pharmaceutically acceptable salt or solvate thereof, is administered to a subject during or as soon as possible after the onset of the symptoms. In specific embodiments, a PPARδ agonist (e.g. Compound 1), or a pharmaceutically acceptable salt or solvate thereof, is administered as soon as is practicable after the onset of a disease or condition is detected or suspected, and for a length of time necessary for the treatment of the disease. In some embodiments, the length required for treatment varies, and the treatment length is adjusted to suit the specific needs of each subject. For example, in specific embodiments, a PPARδ agonist (e.g. Compound 1), or a pharmaceutically acceptable salt or solvate thereof, or a formulation containing Compound I, or a pharmaceutically acceptable salt or solvate thereof, is administered for at least 2 weeks, about 1 month to about 5 years.

### Exemplary Agents for Use in Combination Therapy

In some embodiments, a PPARδ agonist (e.g. Compound 1, or a pharmaceutically acceptable salt), is administered in combination with one or more additional therapies used for treating fatty acid oxidation disorders.

In certain embodiments, the at least one additional therapy is administered at the same time as a PPARδ agonist (e.g. Compound 1), or a pharmaceutically acceptable salt or solvate thereof. In certain embodiments, the at least one additional therapy is administered less frequently than a PPARδ agonist (e.g. Compound 1), or a pharmaceutically acceptable salt or solvate thereof. In certain embodiments, the at least one additional therapy is administered more frequently than a PPARδ agonist (e.g. Compound 1), or a pharmaceutically acceptable salt or solvate thereof. In certain embodiments, the at least one additional therapy is administered prior to administration of a PPARδ agonist (e.g. Compound 1), or a pharmaceutically acceptable salt or solvate thereof. In certain embodiments, the at least one additional therapy is administered after administration of a PPARδ agonist (e.g. Compound 1), or a pharmaceutically acceptable salt or solvate thereof.

In some embodiments, a PPARδ agonist (e.g. Compound 1, or a pharmaceutically acceptable salt), is administered in combination with ubiquinol, ubiquinone, niacin, riboflavin, creatine, L-carnitine, acetyl-L-carnitine, biotin, thiamine, pantothenic acid, pyridoxine, alpha-lipoic acid, n-heptanoic acid, CoQ10, vitamin E, vitamin C, methylcobalamin, folinic acid, resveratrol, N-acetyl-L-cysteine (NAC), zinc, folinic acid/leucovorin calcium, or a combination thereof.

In some embodiments, a PPARδ agonist (e.g. Compound 1, or a pharmaceutically acceptable salt), is administered in combination with succinic acid, or salt thereof, or trisuccinylglycerol, or salt thereof. In some embodiments, a PPARδ agonist (e.g. Compound 1, or a pharmaceutically acceptable salt), is administered in combination with a compound described in International PCT publication no. WO 2017/184583.

In some embodiments, a PPARδ agonist (e.g. Compound 1, or a pharmaceutically acceptable salt), is administered in combination with an antioxidant.

In some embodiments, a PPARδ agonist (e.g. Compound 1, or a pharmaceutically acceptable salt), is administered in combination with an odd-chain fatty acid, odd-chain fatty ketone, L-carnitine, or combinations thereof.

In some embodiments, a PPARδ agonist (e.g. Compound 1, or a pharmaceutically acceptable salt), is administered in combination with triheptanoin, n-heptanoic acid, a triglyceride, or a salt or thereof, or combinations thereof.

In some embodiments, a PPARδ agonist is administered in combination with a Nicotinamide Adenine Dinucleotide (NAD+) pathway modulator. NAD+ plays many important roles within cells, including serving as an oxidizing agent in oxidative phosphorylation which generates ATP from ADP. Increasing cellular concentrations of NAD+ will enhance the oxidative capacity within mitochondria, thereby increasing nutrient oxidation and boost energy supply, which is a primary role of mitochondria. In some embodiments, the NAD+ modulator targets Poly ADP Ribose Polymerase (PARP), Aminocarboxymuconate Semialdehyde Decarboxylase (ACMSD) and N′-Nicotinamide Methyltransferase (NNMT).

### Kits and Articles of Manufacture

Described herein are kits for treating treatment of fatty acid oxidation disorders (FAOD) in an individual comprising administering to said individual a PPARδ agonist (e.g. Compound 1, or a pharmaceutically acceptable salt thereof).

For use in the therapeutic applications described herein, kits and articles of manufacture are also described herein. In some embodiments, such kits include a carrier, package, or container that is compartmentalized to receive one or more containers such as vials, tubes, and the like, each of the container(s) including one of the separate elements to be used in a method described herein. Suitable containers include, for example, bottles, vials, syringes, and test tubes. The containers, in some cases, are formed from a variety of materials such as glass or plastic.

The articles of manufacture provided herein contain packaging materials. Examples of pharmaceutical packaging materials include, but are not limited to, blister packs, bottles, tubes, inhalers, pumps, bags, vials, containers, syringes, bottles, and any packaging material suitable for a selected formulation and intended mode of administration and treatment. A wide array of formulations of the compounds and compositions provided herein are contemplated as are a variety of treatments for any treatment of fatty acid oxidation disorder (FAOD) that benefits from PPARδ modulation.

The container(s) optionally have a sterile access port (for example the container is an intravenous solution bag or a vial having a stopper pierceable by a hypodermic injection needle). Such kits optionally comprise a compound with an identifying description or label or instructions relating to its use in the methods described herein.

A kit will typically include one or more additional containers, each with one or more of various materials (such as reagents, optionally in concentrated form, and/or devices) desirable from a commercial and user standpoint for use of a compound described herein. Non-limiting examples of such materials include, but not limited to, buffers, diluents, filters, needles, syringes; carrier, package, container, vial and/or tube labels listing contents and/or instructions for use, and package inserts with instructions for use. A set of instructions will also typically be included.

In some embodiments, a label is on or associated with the container. A label, in some cases, is on a container when letters, numbers or other characters forming the label are attached, molded or etched into the container itself, a label, in some cases, is associated with a container when it is present within a receptacle or carrier that also holds the container, e.g., as a package insert. A label, in some cases, is used to indicate that the contents are to be used for a specific therapeutic application. The label, in some cases, indicates directions for use of the contents, such as in the methods described herein.

In certain embodiments, a pharmaceutical composition comprising a PPARδ agonist (e.g. Compound 1, or a pharmaceutically acceptable salt thereof), is presented in a pack or dispenser device which, in some cases, contains one or more unit dosage forms. The pack, in some cases, for example contains metal or plastic foil, such as a blister pack. The pack or dispenser device, in some cases, is accompanied by instructions for administration. The pack or dispenser, in some cases, is also accompanied with a notice associated with the container in form prescribed by a governmental agency regulating the manufacture, use, or sale of pharmaceuticals, which notice is reflective of approval by the agency of the form of the drug for human or veterinary administration. Such notice, for example, in some cases, is the labeling approved by the U.S. Food and Drug Administration for prescription drugs, or the approved product insert. Compositions containing a compound provided herein formulated in a compatible pharmaceutical carrier, in some cases, is also prepared, placed in an appropriate container, and labeled for treatment of an indicated condition.

### EXAMPLES

The following examples are provided for illustrative purposes only and not to limit the scope of the claims provided herein.

### Example 1: Cell Lines and Culture

Subjects. Skin biopsies for fibroblast culture are performed on a clinical basis with written informed consent from subjects and/or legal guardians. Fibroblast cells with mutations in any one of the genes and/or proteins associated with a fatty acid oxidation disorder (FAOD) ae obtained from patients' skin biopsies, while wild type (WT) fibroblast cells are obtained from healthy individuals.

Fibroblast cells, in some cases, are obtained from subjects with a confirmed diagnosis of a fatty acid oxidation disorder (FAOD) (e.g. MCAD, VLCAD, CPT1, CACT, CPT2, LCHAD, and/or mitochondrial TFP deficiencies or mutations) or they, in some cases, are purchased is available from commercial sources, e.g. from the Coriell Institute for Medical Research (403 Haddon Avenue, Camden, N.J. 08103).

Cell culture and treatments. Cells are grown in Dulbecco's Modified Eagle Medium (DMEM), Corning Life Sciences, Manassas, Va., containing high glucose levels or in DMEM devoid of glucose for 48-72 hr. Both media are supplemented with fetal bovine serum, glutamine, penicillin and/or streptomycin. In some experiments, fibroblasts are incubated with N-acetylcysteine, resveratrol, mitoQ, Trolox (a hydro-soluble analogue of vitamin E), or bezafibrate, prior to the analysis of parameters.

A PPARδ agonist compound is dissolved in phosphate buffer saline, PBS, as a stock solution. Amounts are added appropriately directly to cell culture media in flasks when the cultures are about 85-90 confluent. The cultures are allowed to grow for 48 h at 37° C., and then harvested. Harvested cell pellets are stored at −80° C. until immune and enzymatic assays analyses. 1 mL to 1.5 mL media samples are also stored at −80° C. for acylcarnitines.

### Example 2: Measurement of Mitochondrial Respiration

Oxygen consumption rate (OCR) is measured with a Seahorse XFe96 Extracellular Flux Analyzer (Sea horse Bioscience, Billerica, Mass.).

Briefly, the apparatus contains a fluoro-phore that is sensitive to changes in oxygen concentration, which enables it to accurately measure the rate at which cytochrome c oxidase (complex IV) reduces one O₂ molecule to two H₂O molecules during OXPHOS. Cells are seeded in 96-well Seahorse tissue culture microplates in growth media at a density of 80,000 cells per well. To ensure equal cell numbers, cells are seeded in cell culture plates pre-coated with Cell-Tak, BD Biosciences, San Jose, Calif. All cell lines are measured with four to eight wells per cell line. Then, the entire set of experiments is repeated. Before running the Seahorse assay, cells are incubated for 1 hour without CO₂ in unbuffered DMEM. Initial OCR is measured to establish a baseline (basal respiration). Maximal respiration is also determined after the injection of 300 nM carbonyl cyanide 4-(trifluoromethoxy)phenylhydrazone (FCCP), Seahorse XF Cell Mito Stress Test Kit, Santa Clara, Calif.

### Example 3: ATP Production Assay

ATP production is determined by a bioluminescence assay using an ATP determination kit (ATPlite kit) from PerkinElmer Inc, Waltham, Mass., according to the manufacturer's instructions.

### Example 4: Western Blotting

Cells are grown in T175 flasks and, at 90-95% confluence, are harvested by trypsinization, pelleted and stored at −80° C. for western blot. Protein content in samples is quantified for data normalization using DC™ Protein Assay kit (Bio-Rad Laboratories).

For cell lysates, pellets are re-suspended in 150-250 μL of RIPA buffer with protease inhibitor cocktail, Roche Diagnostics, Mannheim, Germany. Homogenates are kept on ice for 30 min, shaken every 10 min, and centrifuged. Supernatants are used for western blotting. For mitochondria, pellets are re-suspended in 150-250 μL of 5 mM Tris buffer, pH 7.4, containing 250 mM sucrose, 2 mM EDTA, protease inhibitor cocktail, Roche Diagnostics, Mannheim, Germany, and 0.5 μM trichostatin A, Sigma-Aldrich Co., St. Louis, Mo., homogenized and centrifuged. The pellet is discarded and the supernatant centrifuged. The resulting pellet containing mitochondria is re-suspended in 50 mM Tris buffer, pH 7.4, sonicated and centrifuged again.

Cell lysates or mitochondria are used for western blotting as previously described (Goetzman, E. S. et al. Expression and characterization of mutations in human very long-chain acyl-CoA dehydrogenase using a prokaryotic system. Mol. Genet. Metab. 91, 138-147, (2007)). Briefly, 10 or 20 μg of protein are loaded onto the gel. Following electrophoresis, the gel is blotted onto a nitrocellulose membrane, which is incubated with rabbit anti-ND6 polyclonal antibody (1:100), Santa Cruz Biotechnology, Dallas, Tex., rabbit anti-NDUFV1 polyclonal antibody (1:100), Santa Cruz Biotechnology, Dallas, Tex., rabbit anti-ACAD9 antiserum (1:500), Cocalico Biologicals Inc., PA, rodent anti-total OXPHOS cocktail antibody (1:250), Abcam, Cambridge, Mass., mouse anti-mitofusin 1 (MFN1) monoclonal antibody (1:100), Abcam, Cambridge, Mass., mouse anti-dynamin-related protein 1 (DRP1) monoclonal antibody (1:100), Abcam, Cambridge, Mass., rabbit anti-very long-chain acyl-CoA dehydrogenase (VLCAD) antiserum (1:1,000), Cocalico Biologicals Inc., PA, rabbit anti-voltage-dependent anion channel 1 (VDAC1) monoclonal antibody (1:1,000), Abcam, Cambridge, Mass., mouse anti-glucose-related protein 75 (Grp75) monoclonal antibody (1:250), Abcam, Cambridge, Mass., rabbit anti-glucose-related protein 78 (Grp78) polyclonal antibody (1:250), Abcam, Cambridge, Mass., mouse anti-DNA damage inducible transcript 3 (DDIT3) monoclonal antibody (1:250), Abcam, Cambridge, Mass., goat anti-inositol 1,4,5-trisphosphate receptor 3 (IP3R) polyclonal antibody (1:50), Santa Cruz Biotechnology, Dallas, Tex., or IgG-HRP conjugated antibody, Bio-Rad, Hercules, Calif. Staining of the membranes with Ponceau S, Sigma-Aldrich Co., St. Louis, Mo., or mouse anti-β-actin monoclonal antibody (1:10,000), Sigma-Aldrich Co., St. Louis, Mo., or mouse anti-glyceraldehyde 3-phosphate dehydrogenase (GAPDH) monoclonal antibody (1:15,000), Abcam, Cambridge, Mass., is used to verify equal loading.

### Example 5: Immunofluorescence Microscopy and Mitochondrial Membrane Potential (ΔΨ)

Cells are incubated with the antibodies anti-VLCAD (1:1000), anti-Nrf2 (1:100) or anti-NF-kB (1:1000) at 4° C. overnight. After brief washing with TBST, cells are incubated with donkey anti-rabbit secondary antibody Alexa Fluor 488, from Invitrogen. Nuclei are immunostained with DAPI. The coverslips are then mounted using mounting media before taking images with an Olympus Confocal FluoroView 1000 microscope at a magnification of 60×.

### Example 6: Fatty Acid Oxidation (FAO) Flux Analysis

Fatty acid oxidation (FAO) flux analysis is performed by quantifying the production of ³H₂O from 9,10-[³H]palmitate, PerkinElmer, Waltham, Mass., conjugated to fatty acid-free albumin in fibroblasts cultured in a 24-well plate.

A representative non-limiting example of a FAO flux analysis is described in Bennett, M. J. Assays of fatty acid beta-oxidation activity. Methods Cell Biol 80, 179-197, (2007)). In some embodiments, 300,000 fibroblasts are plated per well in 6-well plates and grown for 24 hours in DMEM with 10% fetal bovine serum. The growth media is then changed to either the same media or devoid of glucose and fibroblasts are grown as described for 48 hr. Subsequently, cells are washed once with PBS and then incubated with 0.34 ρCi [9,10-³H]oleate (45.5 Ci/mmol; Perkin Elmer, Waltham, Mass.) in 50 nmol of oleate prepared in 0.5 mL glucose-free DMEM with 1 μ/ml carnitine and 2 mg/ml α-cyclodextrin for 2 hours at 37° C. Fatty acids are solubilized with α-cyclodextrin as described (Watkins, P. A., Ferrell, E. V. Jr., Pedersen, J. I. & Hoefler, G. Peroxisomal fatty acid beta-oxidation in HepG2 cells. Arch Biochem Biophys 289, 329-336 (1991)). After incubation, ³H₂O released is separated from the oleate on a column containing 750 μL of anion exchange resin (AG 1×8, acetate, 100-200 Mesh, BioRad, Richmond, Calif.) prepared in water. After the incubation medium passes through the column, the plate is washed with 750 μL of water which is also transferred to the column. The resin is then washed twice with 750 μL of water. All eluates are collected in a scintillation vial and mixed with 5 mL of scintillation fluid (Eco-lite, MP), followed by counting in a Beckman scintillation counter in the tritium window. Assays are performed in quadruplicate with triplicate blanks (cell free wells). Standards contain a 50 μL aliquot of the incubation mix with 2.75 mL of deionized water and 5 mL of scintillation fluid.

### Example 7: Cell Viability Assay

Cell viability is evaluated with a 3-(4,5-dimethylthiazol-2-yl)-5-(3-carboxymethoxyphenyl)-2-(4-sulfophenyl)-2H-tetrazolium (MTS) assay kit according to the manufacturer's instructions, Abcam, Cambridge, Mass. The absorbance is read in the FLUOstar Omega plate reader at 490 nm.

### Example 8: Apoptosis Assay

Apoptosis is evaluated with an Alexa Fluor® 488 annexin V/Dead Cell Apoptosis kit according to manufacturer's instructions, Invitrogen, Grand Island, N.Y. The kit contains annexin V labeled with a fluorophore and propidium iodide (PI). Annexin V can identify apoptotic cells by binding to phosphatidylserine exposed on the outer leaflet of cell plasma membrane while PI stains dead cells by binding to nucleic acids. Fluorescence is determined in a Becton Dickinson FACSAria II flow cytometer, BD Biosciences, San Jose, Calif.

### Example 9: Determination of Acylcarnitine Levels

Acylcarnitine analysis is performed utilizing the appropriate tandem mass spectrometry (MS/MS) protocols.

### Example 10: ETF Fluorescence Reduction ACAD Activity Assay

Enzyme assays used to measure ACAD enzyme activity at the picomoles level in tissues and in cell culture have been described. An assay protocol with the key ingredient being ETF (electron transfer flavoprotein) that is isolated from pig liver has been published (Vockley et al., Mammalian branched-chain acyl-CoA dehydrogenases: molecular cloning and characterization of recombinant enzymes, Methods Enzymol. 2000; 324:241-58; which is incorporated by reference for such assay).

### Example 11: Measurement of the Level of Expression of VLCAD

The effect of increasing amounts of PPARδ agonist compound on ACADVL gene expression in VLCAD deficient or mutated cells is monitored using standard qRT-PCR protocol. Messenger RNA transcription levels of ACADVL (MIM: 609575) for the patient's fibroblasts cell lines with VLCAD deficiency untreated and treated with PPARδ agonist compound are quantified via qRT-PCR with an Applied Biosystems StepOnePlus instrument using TaqMan™ Gene Expression Master Mix (from ThermoFisher Scientific). The reference sample is fibroblasts with no VLCAD deficiency. Human GAPDH is used as an endogenous control. Commercial primers for ACADVL and GAPDH are used using and TaqMan™ Gene Expression Assay (ThermoFisher Scientific), which consists of a pair of unlabeled PCR primers and a TaqMan probe with a FAM™ or VIC(R) dye label on the 5′-end and minor groove binder (MGB) and non-fluorescent quencher (NFQ) on the 3-end. The relative quantity RQ of the samples is compared between the reference sample, treated VLCAD deficiency cell lines untreated and treated with PPARδ agonist compound.

### Example 12: Combination Therapy

PPARδ agonists can be used in combination with other therapies for fatty acid oxidation disorders (FAOD). In some embodiments, a PPARδ agonist compound is administered to an individual with a FAOD in combination with one or more of the following: ubiquinol, ubiquinone, niacin, riboflavin, creatine, L-carnitine, acetyl-L-carnitine, biotin, thiamine, pantothenic acid, pyridoxine, alpha-lipoic acid, n-heptanoic acid, triheptanoin, a triglyceride, or a salt or thereof, CoQ10, vitamin E, vitamin C, methylcobalamin, folinic acid, N-acetyl-L-cysteine (NAC), zinc, folinic acid/leucovorin calcium.

Combination therapy is advantageous when efficacy is greater than either agent alone or when the dose required for either drug is reduced thereby improving the side effect profile.

### Example 13: Clinical Trial for Fatty Acid Oxidation Disorder

A non-limiting example of a fatty acid oxidation disorder (FAOD) clinical trial in humans is described below.

Purpose: The purposes of this study are: to assess the safety and tolerability of 12 weeks treatment with Compound 1, or a pharmaceutically acceptable salt or solvate thereof, in subjects with FAOD; to investigate pharmacokinetics of Compound 1, or a pharmaceutically acceptable salt or solvate thereof, in subjects with FAOD treated with Compound 1, or a pharmaceutically acceptable salt or solvate thereof; to investigate the pharmacodynamics effects of Compound 1, or a pharmaceutically acceptable salt or solvate thereof, in subjects with FAOD treated with Compound 1, or a pharmaceutically acceptable salt or solvate thereof.

Intervention: Patients are administered 10-2000 mg of Compound 1, or a pharmaceutically acceptable salt or solvate thereof, per day as single agent or in combination. In one cohort, subjects will receive 50 mg of Compound 1, or a pharmaceutically acceptable salt or solvate thereof, once daily for a total of 12 weeks. In another cohort, subjects will receive 100 mg of Compound 1, or a pharmaceutically acceptable salt or solvate thereof, once daily for a total of 12 weeks. Other cohorts are contemplated.

Compound 1, or a pharmaceutically acceptable salt or solvate thereof, will be packed in bottles as capsules.

Detailed Description: Patients will be given Compound 1, or a pharmaceutically acceptable salt or solvate thereof, orally once a day.

Eligibility: 18 years and older with FAOD.

Inclusion Criteria: Confirmed diagnosis of one of the following: carnitine palmitoyltransferase II deficiency (CPT2), very long-chain Acyl-CoA dehydrogenase deficiency (VLCAD), long-chain 3-hydroxyacyl-CoA dehydrogenase deficiency (LCHAD), or trifunctional protein deficiency (TFP).

A diagnostic acylcarnitine profile, in blood or cultured fibroblasts.

Genotyping with at least 1 allele that is not a stop codon or a frame shift.

Have evidence of any one of the following clinical manifestations despite therapy: Chronic elevated Creatine Kinase (CPK) as evidenced by at least 2 blood CPK levels above the ULN obtained at least 3 months apart, history of cardiomyopathy, a clinical event of hypoglycemia, rhabdomyolysis, or exacerbation of cardiomyopathy within the 12 months preceding enrollment.

Currently following a stable dietary regimen with avoidance of fasting as documented by a 3-day dietary record obtained during the screening period.

A stable treatment regimen for at least 30 days prior to enrollment.

Expected and willing to remain on stable diet and medication through the study.

Ambulatory and able to perform the study exercise tests.

Adequate kidney function defined as an estimated glomerular filtration rate (eGFR)≥60 mL/min/1.73 m2 using the Cockcroft-Gault formula.

Able to swallow capsules.

Exclusion Criteria: Subjects presenting with any of the following will not be included in the study:
unstable or poorly controlled disease as determined by one or more of the following: echocardiogram with evidence of active or worsening cardiomyopathy at screening; presence of symptoms of acute rhabdomyolysis with elevations in serum CPK consistent with acute exacerbation of myopathy; evidence of acute crisis from their underlying disease.
currently taking anticoagulants.
have motor abnormalities other than those related to the fatty acid oxidation disorder that could interfere with the outcome measures.
treatment with an investigational drug within 1 month or within 5 half-lives, whichever is longer.
evidence of significant concomitant clinical disease that in the opinion of the Investigator may need a change in management during the study or could interfere with the conduct or safety of this study. (Stable well-controlled chronic conditions such as controlled hypertension (BP<140/90 mmHg) thyroid disease, well-controlled Type 1 or Type 2 diabetes (HbA1c<8%), hypercholesterolemia, gastroesophageal reflux, or depression under control with medication (other than tricyclic antidepressants), are acceptable provided the symptoms and medications would not be predicted to compromise safety or interfere with the tests and interpretations of this study).
history of cancer with the exception of in situ skin cancer.
have been hospitalized within the 3 months prior to screening for any major medical condition (as deemed by the primary investigator).
any condition possibly reducing drug absorption (e.g., gastrectomy).
history of clinically significant liver disease as evidenced by elevations in ALT, GGT or TB.
positive hepatitis B surface antigen (HBsAg) or hepatitis C, or HIV at screening.
history of regular alcohol consumption exceeding 14 drinks/week (1 drink=150 mL of wine or 360 mL of beer or 45 mL of spirits) within 6 months of screening.
any other severe acute or chronic medical or psychiatric condition or laboratory abnormality that in the opinion of the Investigator may increase the risk associated with study participation or investigational product administration or may interfere with the interpretation of study results.

Primary Outcome Measures: Safety Endpoints include: number and severity of adverse events. Absolute values, changes from baseline at Week 12 and incidence of clinically significant changes in: laboratory safety tests; electrocardiograms; supine vital signs; evaluation of events of special interest (rhabdomyolysis) and clinically significant changes in laboratory parameters of muscle injury including total CPK, adolase, and cardiac specific troponin (cTn).

Pharmacokinetic Endpoints include: Compound 1 plasma concentrations and identification of metabolites using pooled plasma.

Pharmacodynamic Endpoints include: Absolute values and changes from baseline at Week 12 in: whole body fatty acid oxidation (¹³CO₂ production) and blood acylcarnitines (UHPLC-MS/MS method).

Secondary Outcome Measures: To assess the change from baseline following 12 weeks of treatment with Compound 1, or a pharmaceutically acceptable salt or solvate thereof, in: submaximal treadmill exercise tolerance; distance walked during a 12 minute walk test; 36-Item Short Form Survey (SF-36) total score and subscales (questions 3-12). Change from baseline in Fatigue Impact Scale score (every visit). Change from baseline in Brief Pain Inventory (short form) (every visit). Blood inflammatory cytokines (Multiplex Immunoassay for sE-Selectin; GM-CSF; ICAM-1/CD54; IFN alpha; IFN gamma; IL-1 alpha; IL-1 beta; TL-4; IL-6; IL-8; IL-10; IL-12p70; IL-13; IL-17A/CTLA-8; IP-10/CXCL10; MCP-1/CCL2; MIP-1alpha/CCL3; MIP-1 beta/CCL4; sP-Selectin; TNF alpha).

FAOD Clinical Trial Results with Compound 1

In general, Compound 1 was well tolerated among subjects that participated in the study.

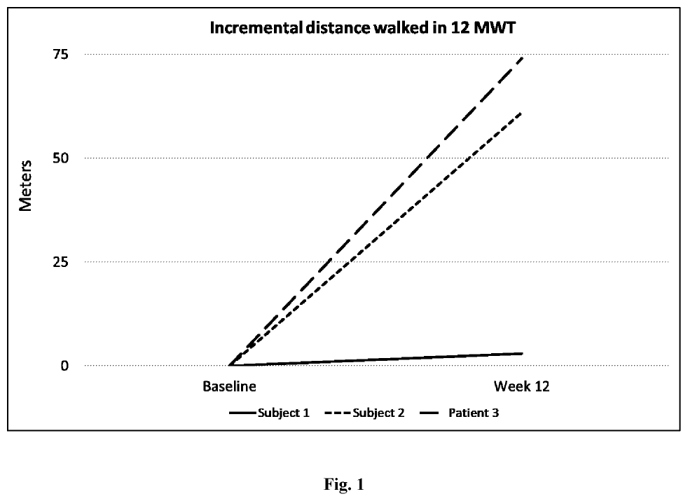

Improvements in exercise capacity was observed in subjects that received 50 mg of Compound 1, or a pharmaceutically acceptable salt or solvate thereof, once daily for a total of 12 weeks. Subjects were able to increase the distance walked during a 12-minute walk test. FIG. 1 shows the results of the impact of Compound 1 on the 12-minute walk test in this group of subjects. In this same group of subjects, decreases in heart rate were observed during the last ten minutes of exercise.

A trend towards increases in exhaled ¹³CO₂ was observed in subjects that received 50 mg of Compound 1, or a pharmaceutically acceptable salt or solvate thereof, once daily for a total of 12 weeks.

The examples and embodiments described herein are for illustrative purposes only and various modifications or changes suggested to persons skilled in the art are to be included within the spirit and purview of this application and scope of the appended claims.

### Example 14: Sequences

TABLE 1
Carnitine Shuttle Genes

| SEQ | | NCBI | |
|---|---|---|---|
| ID | | Reference | |
| NO | Gene | Number | Nucleotide Sequence |
| 1 | CPT1A | NM_ | GGACCCGCCTCAGCCAATCCGCTGCTGCCGGCGTCGGGTGC |
| | | 001031847.2 | GCTCGGCCTCGCCCGCGGCCCTCCTTCCCCGGCTCCCGCTCG |
| | | | CCGCTCGTTCACTCCACCGCCGCCGCCGCCGCCGCCGCTGC |
| | | | CGCTGCCGCTGCCGCACCTCCGTAGCTGACTCGGTACTCTCT |
| | | | GAAGATGGCAGAAGCTCACCAAGCTGTGGCCTTTCAGTTCA |
| | | | CGGTCACTCCGGACGGGATTGACCTGCGGCTGAGCCATGAA |
| | | | GCTCTTAGACAAATCTATCTCTCTGGACTTCATTCCTGGAAA |
| | | | AAGAAGTTCATCAGATTCAAGAACGGCATCATCACTGGCGT |
| | | | GTACCCGGCAAGCCCCTCCAGTTGGCTTATCGTGGTGGTGG |
| | | | GCGTGATGACAACGATGTACGCCAAGATCGACCCCTCGTTA |
| | | | GGAATAATTGCAAAAATCAATCGGACTCTGGAAACGGCCA |
| | | | ACTGCATGTCCAGCCAGACGAAGAACGTGGTCAGCGGCGT |
| | | | GCTGTTTGGCACCGGCCTGTGGGTGGCCCTCATCGTCACCA |
| | | | TGCGCTACTCCCTGAAAGTGCTGCTCTCCTACCACGGGTGG |
| | | | ATGTTCACTGAGCACGGCAAGATGAGTCGTGCCACCAAGAT |
| | | | CTGGATGGGTATGGTCAAGATCTTTTCAGGCCGAAAACCCA |
| | | | TGTTGTACAGCTTCCAGACATCGCTGCCTCGCCTGCCGGTCC |
| | | | CGGCTGTCAAAGACACTGTGAACAGGTATCTACAGTCGGTG |
| | | | AGGCCTCTTATGAAGGAAGAAGACTTCAAACGGATGACAG |
| | | | CACTTGCTCAAGATTTTGCTGTCGGTCTTGGACCAAGATTAC |
| | | | AGTGGTATTTGAAGTTAAAATCCTGGTGGGCTACAAATTAC |
| | | | GTGAGCGACTGGTGGGAGGAGTACATCTACCTCCGAGGAC |
| | | | GAGGGCCGCTCATGGTGAACAGCAACTATTATGCCATGGAT |
| | | | CTGCTGTATATCCTTCCAACTCACATTCAGGCAGCAAGAGC |
| | | | CGGCAACGCCATCCATGCCATCCTGCTTTACAGGCGCAAAC |
| | | | TGGACCGGGAGGAAATCAAACCAATTCGTCTTTTGGGATCC |
| | | | ACGATTCCACTCTGCTCCGCTCAGTGGGAGCGGATGTTTAA |
| | | | TACTTCCCGGATCCCAGGAGAGGAGACAGACACCATCCAGC |
| | | | ACATGAGAGACAGCAAGCACATCGTCGTGTACCATCGAGG |
| | | | ACGCTACTTCAAGGTCTGGCTCTACCATGATGGGCGGCTGC |
| | | | TGAAGCCCCGGGAGATGGAGCAGCAGATGCAGAGGATCCT |
| | | | GGACAATACCTCGGAGCCTCAGCCCGGGGAGGCCAGGCTG |
| | | | GCAGCCCTCACCGCAGGAGACAGAGTTCCCTGGGCCAGGTG |
| | | | TCGTCAGGCCTATTTTGGACGTGGGAAAAATAAGCAGTCTC |
| | | | TTGATGCTGTGGAGAAAGCAGCGTTCTTCGTGACGTTAGAT |
| | | | GAAACTGAAGAAGGATACAGAAGTGAAGACCCGGATACGT |
| | | | CAATGGACAGCTACGCCAAATCTCTACTACACGGCCGATGT |
| | | | TACGACAGGTGGTTTGACAAGTCGTTCACGTTTGTTGTCTTC |
| | | | AAAAACGGGAAGATGGGCCTCAACGCTGAACACTCCTGGG |
| | | | CAGATGCGCCGATCGTGGCCCACCTTTGGGAGTACGTCATG |
| | | | TCCATTGACAGCCTCCAGCTGGGCTATGCGGAGGATGGGCA |
| | | | CTGCAAAGGCGACATCAATCCGAACATTCCGTACCCCACCA |
| | | | GGCTGCAGTGGGACATCCCGGGGGAATGTCAAGAGGTTAT |
| | | | AGAGACCTCCCTGAACACCGCAAATCTTCTGGCAAACGACG |
| | | | TGGATTTCCATTCCTTCCCATTCGTAGCCTTTGGTAAAGGAA |
| | | | TCATCAAGAAATGTCGCACGAGCCCAGACGCCTTTGTGCAG |
| | | | CTGGCCCTCCAGCTGGCGCACTACAAGGACATGGGCAAGTT |
| | | | TTGCCTCACATACGAGGCCTCCATGACCCGGCTCTTCCGAG |
| | | | AGGGGAGGACGGAGACCGTGCGCTCCTGCACCACTGAGTC |
| | | | ATGCGACTTCGTGCGGGCCATGGTGGACCCGGCCCAGACGG |
| | | | TGGAACAGAGGCTGAAGTTGTTCAAGTTGGCGTCTGAGAAG |
| | | | CATCAGCATATGTATCGCCTCGCCATGACCGGCTCTGGGAT |
| | | | CGATCGTCACCTCTTCTGCCTTTACGTGGTGTCTAAATATCT |
| | | | CGCTGTGGAGTCCCCTTTCCTTAAGGAAGTTTTATCTGAGCC |
| | | | TTGGAGATTATCAACAAGCCAGACCCCTCAGCAGCAAGTGG |
| | | | AGCTGTTTGACTTGGAGAATAACCCAGAGTACGTGTCCAGC |
| | | | GGAGGGGGCTTTGGACCGGTTGCTGATGACGGCTATGGTGT |
| | | | GTCGTACATCCTTGTGGGAGAGAACCTCATCAATTTCCACA |
| | | | TTTCTTCCAAGTTCTCTTGCCCTGAGACGGGGATTATAAGTC |
| | | | AAGGACCAAGTTCAGATACTTGAGACAAAGTGGAAAGTCT |
| | | | CAGCATATGGAAACAAGGCCTTGGAGGAGACCATGGACAT |
| | | | CACCAAGTTCATGTGCTGGGCTGGAAAGAAAAGCCTGTTGA |
| | | | TTTTCACTTGCTGTGCATTTATTCATCCATTCCATTGCCTCAA |
| | | | TGCTGAGAACAGTGCCTGACACATAAAAGATGCTCAATAAA |
| | | | TATGTTAAAAGTAAAAAAAAAAAAAAAAAAAAAAAAAAA |
| | | | AAAAAAAAA |
| 2 | CPT1B | NM_004377.3 | GGGGGTGGCTAGGCCTGAAGGACGTGGGGACACGGGCCAG |
| | | | AGTGGCTGGCCCCACGCACGGACAGGAGTGAACCCGAGCT |
| | | | GTGCCGACCAACCCCCAGGATGGCGGAAGCTCACCAGGCC |
| | | | GTGGCCTTCCAGTTCACGGTGACCCCAGACGGGGTCGACTT |
| | | | CCGGCTCAGTCGGGAGGCCCTGAAACACGTCTACCTGTCTG |
| | | | GGATCAACTCCTGGAAGAAACGCCTGATCCGCATCAAGAAT |
| | | | GGCATCCTCAGGGGCGTGTACCCTGGCAGCCCCACCAGCTG |
| | | | GCTGGTCGTCATCATGGCAACAGTGGGTTCCTCCTTCTGCA |
| | | | ACGTGGACATCTCCTTGGGGCTGGTCAGTTGCATCCAGAGA |
| | | | TGCCTCCCTCAGGGGTGTGGCCCCTACCAGACCCCGCAGAC |
| | | | CCGGGCACTTCTCAGCATGGCCATCTTCTCCACGGGCGTCT |
| | | | GGGTGACGGGCATCTTCTTCTTCCGCCAAACCCTGAAGCTG |
| | | | CTTCTCTGCTACCATGGGTGGATGTTTGAGATGCATGGCAA |
| | | | GACCAGCAACTTGACCAGGATCTGGGCTATGTGTATCCGCC |
| | | | TTCTATCCAGCCGGCACCCTATGCTCTACAGCTTCCAGACAT |
| | | | CTCTGCCCAAGCTTCCTGTGCCCAGGGTGTCAGCCACAATT |
| | | | CAGCGGTACCTAGAGTCTGTGCGCCCCTTGTTGGATGATGA |
| | | | GGAATATTACCGCATGGAGTTGCTGGCCAAAGAATTCCAGG |
| | | | ACAAGACTGCCCCCAGGCTGCAGAAATACCTGGTGCTCAAG |
| | | | TCATGGTGGGCAAGTAACTATGTGAGTGACTGGTGGGAAGA |
| | | | GTACATCTACCTTCGAGGCAGGAGCCCTCTCATGGTGAACA |
| | | | GCAACTATTATGTCATGGACCTTGTGCTCATCAAGAATACA |
| | | | GACGTGCAGGCAGCCCGCCTGGGAAACATCATCCACGCCAT |
| | | | GATCATGTATCGCCGTAAACTGGACCGTGAAGAAATCAAGC |
| | | | CTGTGATGGCACTGGGCATAGTGCCTATGTGCTCCTACCAG |
| | | | ATGGAGAGGATGTTCAACACCACTCGGATCCCGGGCAAGG |
| | | | ACACAGATGTGCTACAGCACCTCTCAGACAGCCGGCACGTG |
| | | | GCTGTCTACCACAAGGGACGCTTCTTCAAGCTGTGGCTCTA |
| | | | TGAGGGCGCCCGTCTGCTCAAGCCTCAGGATCTGGAGATGC |
| | | | AGTTCCAGAGGATCCTGGACGACCCCTCCCCACCTCAGCCT |
| | | | GGGGAGGAGAAGCTGGCAGCCCTCACTGCAGGAGGAAGGG |
| | | | TGGAGTGGGCGCAGGCACGCCAGGCCTTCTTTAGCTCTGGA |
| | | | AAGAATAAGGCTGCCTTGGAGGCCATCGAGCGTGCCGCTTT |
| | | | CTTCGTGGCCCTGGATGAGGAATCCTACTCCTATGACCCCG |
| | | | AAGATGAGGCCAGCCTCAGCCTCTATGGCAAGGCCCTGCTA |
| | | | CATGGCAACTGCTACAACAGGTGGTTTGACAAATCCTTCAC |
| | | | TCTCATTTCCTTCAAGAATGGCCAGTTGGGTCTCAATGCAG |
| | | | AGCATGCGTGGGCAGATGCTCCCATCATTGGGCACCTCTGG |
| | | | GAGTTTGTCCTGGGCACAGACAGCTTCCACCTGGGCTACAC |
| | | | GGAGACCGGGCACTGCCTGGGCAAACCGAACCCTGCGCTC |
| | | | GCACCTCCTACACGGCTGCAGTGGGACATTCCAAAACAGTG |
| | | | CCAGGCGGTCATCGAGAGTTCCTACCAGGTGGCCAAGGCGT |
| | | | TGGCAGACGACGTGGAGTTGTACTGCTTCCAGTTCCTGCCC |
| | | | TTTGGCAAAGGCCTCATCAAGAAGTGCCGGACCAGCCCTGA |
| | | | TGCCTTTGTGCAGATCGCGCTGCAGCTGGCTCACTTCCGGG |
| | | | ACAGGGGTAAGTTCTGCCTGACCTATGAGGCCTCAATGACC |
| | | | AGAATGTTCCGGGAGGGACGGACTGAGACTGTGCGTTCCTG |
| | | | TACCAGCGAGTCCACAGCCTTTGTGCAGGCCATGATGGAGG |
| | | | GGTCCCACACAAAAGCAGACCTGCGAGATCTCTTCCAGAAG |
| | | | GCTGCTAAGAAGCACCAGAATATGTACCGCCTGGCCATGAC |
| | | | CGGGGCAGGGATCGACAGGCACCTCTTCTGCCTTTACTTGG |
| | | | TCTCCAAGTACCTAGGAGTCAGCTCTCCTTTCCTTGCTGAGG |
| | | | TGCTCTCGGAACCCTGGCGTCTCTCCACCAGCCAGATCCCC |
| | | | CAATCCCAGATCCGCATGTTCGACCCAGAGCAGCACCCCAA |
| | | | TCACCTGGGCGCTGGAGGTGGCTTTGGCCCTGTAGCAGATG |
| | | | ATGGCTATGGAGTTTCCTACATGATTGCAGGCGAGAACACG |
| | | | ATCTTCTTCCACATCTCCAGCAAGTTCTCAAGCTCAGAGAC |
| | | | GAACGCCCAGCGCTTTGGAAACCACATCCGCAAAGCCCTGC |
| | | | TGGACATTGCTGATCTTTTCCAAGTTCCCAAGGCCTACAGCT |
| | | | GAAGGTTGGAGAAATGCCAGCTGCCCTTTCGTCCCCACACT |
| | | | GTGGAGGAAGGGACCTGTGGCAGCTCACAGGCATGAGGGG |
| | | | TGGCCGTGCACAGGTGCCCAGGCTCCAAGGACAGCTCCGGC |
| | | | AGCAGGTCCTCGCTGGGCAGATGCTGCTCCCTGAGGGCCCA |
| | | | GGTGGTGGAGGTGGGGTTGGAGCAGGAAGGGAATTTTGAT |
| | | | TTTTTTTTTTCTTGATAGATACTAATAAAAATAAGGCTGTGT |
| | | | AATTTTCTCTCAGCCCTTAGGTACCTGTGTTTTGTTTGGGAA |
| | | | CTCGGAGGCCCTCCCCCTCCCCCAGCTCAGACCACAGAGGT |
| | | | GGCAAGAGAAGGGCTGAAGCTGGAAGACTGTTCATGAGGG |
| | | | ACTTGTGTGACCTGCTTTGAAATGTGTGACTCTGCTGAGTGA |
| | | | CGTAGGCTCTGAGATAGCTGTCCACGCCCACGTGTTTGCTT |
| | | | GGAATAAATACTTGCCTCAGAACCTTCAAAAAAAAAAAAA |
| | | | AAAAA |
| 3 | SLC25A20 | NM_000387.6 | GAAAGGTCGGCGGCGCCGGCACTGCAGCTGGGGCTGAGAA |
| | | | GCCAGGACGGCCCGAGAACTGACAGACGGAGTGACAGACG |
| | | | GACTGACCATGGCCGACCAGCCAAAACCCATCAGCCCGCTC |
| | | | AAGAACCTGCTGGCCGGCGGCTTTGGCGGCGTGTGCCTGGT |
| | | | GTTCGTCGGTCACCCTCTGGACACGGTCAAGGTCCGACTGC |
| | | | AGACACAGCCACCGAGTTTGCCTGGACAACCTCCCATGTAC |
| | | | TCTGGGACCTTTGACTGTTTCCGGAAGACTCTTTTTAGAGAG |
| | | | GGCATCACGGGGCTATATCGGGGAATGGCTGCCCCTATCAT |
| | | | CGGGGTCACTCCCATGTTTGCCGTGTGCTTCTTTGGGTTTGG |
| | | | TTTGGGGAAGAAACTACAACAGAAACACCCAGAAGATGTG |
| | | | CTCAGCTATCCCCAGCTTTTTGCAGCTGGGATGTTATCTGGC |
| | | | GTATTCACCACAGGAATCATGACTCCTGGAGAACGGATCAA |
| | | | GTGCTTATTACAGATTCAGGCTTCTTCAGGAGAAAGCAAGT |
| | | | ACACTGGTACCTTGGACTGTGCAAAGAAGCTGTACCAGGAG |
| | | | TTTGGGATCCGAGGCATCTACAAAGGGACTGTGCTTACCCT |
| | | | TATGCGAGATGTCCCAGCTAGTGGAATGTATTTCATGACAT |
| | | | ATGAATGGCTGAAAAATATCTTCACTCCGGAGGGAAAGAG |
| | | | GGTCAGTGAGCTCAGTGCCCCTCGGATCTTGGTGGCTGGGG |
| | | | GCATTGCAGGGATCTTCAACTGGGCTGTGGCAATCCCCCCA |
| | | | GATGTGCTCAAGTCTCGATTCCAGACTGCACCTCCTGGGAA |
| | | | ATATCCTAATGGTTTCAGAGATGTGCTGAGGGAGCTGATCC |
| | | | GGGATGAAGGAGTCACATCCTTGTACAAAGGGTTCAATGCA |
| | | | GTGATGATCCGAGCCTTCCCAGCCAATGCGGCCTGTTTCCTT |
| | | | GGCTTTGAAGTTGCCATGAAGTTCCTTAATTGGGCCACCCC |
| | | | CAACTTGTGAGGCTGAAGGCTGCTCAAGTTCACTTCTGGAT |
| | | | GCTGGAAGCTGTCGTTGAGGAGAAGGAGTAGTAAGCAGAA |
| | | | CTAAGCAGTCTTGGAGGGCAAGGGGAGGGGAATGGTGAGA |
| | | | TCCGAGCCCTGTGCATGGACTTGGTGAGACTGTTGCCTTAA |
| | | | TGACATCCTGCACCGTGTATAACTTAGTGTGTCATTTTGAAA |
| | | | CTTGAATTCATTCTTATCAATTTAAGGGATCTTAAAAGGATT |
| | | | TGGAAATGGAACAAGTAGCTTCCAGACCAGATACTACCTGT |
| | | | GGCAAGAATGCTGCCTACCAGTTAACTGCTGGTCCTACCAC |
| | | | AGTCAAAGTATTCCTCATTAAAGAGAGAATCTCAGGTTCTC |
| | | | ACTGGAGGCACTGTGCATATTTTCAACCAGATCACCAGGAG |
| | | | CTGAGATCTTCTTCAGTCCCTAGCCAGGAATACCCATTTGAT |
| | | | TTCCAGGGTGCCATCTAATCCTGGGCTGTACATGTGGATAT |
| | | | GGACTTGAGGCCCACCTCTGTGTCCAAGTGGATTGAGCATA |
| | | | TATGCCTAGGAGGAGATAGACTGTTAATCGTTGGATTTTGA |
| | | | TTTTTTTTTTTTATGCCTGCAAATAATCAAAAGTAAAACTGG |
| | | | AGTAGCCTAATTTTCTGGGAGCAGGTGGAGAACTTTCCCTC |
| | | | CTACACAGTGAGGACAGTCCCAGTCTGCTGGGATAAGTGAG |
| | | | AAAGCCCAGGGTGTAGGAAGGCCCTTTTTACATACTCTTTT |
| | | | CTCATGAGAGCTCACTATTTTAACAATAAACAATAAACGTT |
| | | | GTTTCTAATTTTT |
| 4 | CPT2 | NM_000098.3 | GGAGAAGTGCCTCAGGAGTCCTGACGCAGTGTCTTGGGCGC |
| | | | TAACGGCGGCGGCGGCCTTGTGTTTAGACTCCAGAACTCCC |
| | | | CACTTGCCGCGTTCTCGCCGCCGCAGGCTCCCGGGACGATG |
| | | | GTGCCCCGCCTGCTGCTGCGCGCCTGGCCCCGGGGCCCCGC |
| | | | GGTTGGTCCGGGAGCCCCCAGTCGGCCCCTCAGCGCCGGCT |
| | | | CCGGGCCCGGCCAGTACCTGCAGCGCAGCATCGTGCCCACC |
| | | | ATGCACTACCAGGACAGCCTGCCCAGGCTGCCTATTCCCAA |
| | | | ACTTGAAGACACCATTAGGAGATACCTCAGTGCACAGAAGC |
| | | | CTCTCTTGAATGATGGCCAGTTCAGGAAAACAGAACAATTT |
| | | | TGCAAGAGTTTTGAAAATGGGATTGGAAAAGAACTGCATG |
| | | | AGCAGCTGGTTGCTCTGGACAAACAGAATAAACATACAAG |
| | | | CTACATTTCGGGACCCTGGTTTGATATGTACCTATCTGCTCG |
| | | | AGACTCCGTTGTTCTGAACTTTAATCCATTTATGGCTTTCAA |
| | | | TCCTGACCCAAAATCTGAGTATAATGACCAGCTCACCCGGG |
| | | | CAACCAACATGACTGTTTCTGCCATCCGGTTTCTGAAGACA |
| | | | CTCCGGGCTGGCCTTCTGGAGCCAGAAGTGTTCCACTTGAA |
| | | | CCCTGCAAAAAGTGACACTATCACCTTCAAGAGACTCATAC |
| | | | GCTTTGTGCCTTCCTCTCTGTCCTGGTATGGGGCCTACCTGG |
| | | | TCAATGCGTATCCCCTGGATATGTCCCAGTATTTTCGGCTTT |
| | | | TCAACTCAACTCGTTTACCCAAACCCAGTCGGGATGAACTC |
| | | | TTCACTGATGACAAGGCCAGACACCTCCTGGTCCTAAGGAA |
| | | | AGGAAATTTTTATATCTTTGATGTCCTGGATCAAGATGGGA |
| | | | ACATTGTGAGCCCCTCGGAAATCCAGGCACATCTGAAGTAC |
| | | | ATTCTCTCAGACAGCAGCCCCGCCCCCGAGTTTCCCCTGGC |
| | | | ATACCTGACCAGTGAGAACCGAGACATCTGGGCAGAGCTC |
| | | | AGGCAGAAGCTGATGAGTAGTGGCAATGAGGAGAGCCTGA |
| | | | GGAAAGTGGACTCGGCAGTGTTCTGTCTCTGCCTAGATGAC |
| | | | TTCCCCATTAAGGACCTTGTCCACTTGTCCCACAATATGCTG |
| | | | CATGGGGATGGCACAAACCGCTGGTTTGATAAATCCTTTAA |
| | | | CCTCATTATCGCCAAGGATGGCTCTACTGCCGTCCACTTTGA |
| | | | GCACTCTTGGGGTGATGGTGTGGCAGTGCTCAGATTTTTTA |
| | | | ATGAAGTATTTAAAGACAGCACTCAGACCCCTGCCGTCACT |
| | | | CCACAGAGCCAGCCAGCTACCACTGACTCTACTGTCACGGT |
| | | | GCAGAAACTCAACTTCGAGCTGACTGATGCCTTAAAGACTG |
| | | | GCATCACAGCTGCTAAGGAAAAGTTTGATGCCACCATGAAA |
| | | | ACCCTCACTATTGACTGCGTCCAGTTTCAGAGAGGAGGCAA |
| | | | AGAATTCCTGAAGAAGCAAAAGCTGAGCCCTGACGCAGTT |
| | | | GCCCAGCTGGCATTCCAGATGGCCTTCCTGCGGCAGTACGG |
| | | | GCAGACAGTGGCCACCTACGAGTCCTGTAGCACTGCCGCAT |
| | | | TCAAGCACGGCCGCACTGAGACCATCCGCCCGGCCTCCGTC |
| | | | TATACAAAGAGGTGCTCTGAGGCCTTTGTCAGGGAGCCCTC |
| | | | CAGGCACAGTGCTGGTGAGCTTCAGCAGATGATGGTTGAGT |
| | | | GCTCCAAGTACCATGGCCAGCTGACCAAAGAAGCAGCAAT |
| | | | GGGCCAGGGCTTTGACCGACACTTGTTTGCTCTGCGGCATC |
| | | | TGGCAGCAGCCAAAGGGATCATCTTGCCTGAGCTCTACCTG |
| | | | GACCCTGCATACGGGCAGATAAACCACAATGTCCTGTCCAC |
| | | | GAGCACACTGAGCAGCCCAGCAGTGAACCTTGGGGGCTTTG |
| | | | CCCCTGTGGTCTCTGATGGCTTTGGTGTTGGGTATGCTGTTC |
| | | | ATGACAACTGGATAGGCTGCAATGTCTCTTCCTACCCAGGC |
| | | | CGCAATGCCCGGGAGTTTCTCCAATGTGTGGAGAAGGCCTT |
| | | | AGAAGACATGTTTGATGCCTTAGAAGGCAAATCCATCAAAA |
| | | | GTTAACTTCTGGGCAGATGAAAAGCTACCATCACTTCCTCA |
| | | | TCATGAAAACTGGGAGGCCGGGCATGGTGGCTCATGCCTGT |
| | | | AATCCCAGCATTTTGAGAGGCTGAGGCGGGTGGATCACTTG |
| | | | AGGTCAGGAGTTTGAGACCAACCTGGCCAACATGGTGAAA |
| | | | CCTTGTCTCTACTAAAAATACAAAAATTAGCTGGGTGTGGT |
| | | | GGCATGTGCCTATAATCCCAGCTACTTGGGAGGTTGAAGCA |
| | | | GAATTGCTTGAACCCAGGAGGTGGAGGTTGCAGTGAGCTGA |
| | | | GATCACACCACTGCACTCCGGCCTGGGCGACAGAGCGAGA |
| | | | CTGTCTCAAAAAAACAAAAAAGAAAAAAAAACTGGGGCCT |
| | | | GTGTAGCCAGTGGGTGCTATTCTGTGAAACTAATCATAAGC |
| | | | TGCCTAGGCAGCCAGCTACAGGCTTGAGCTTTAAATTCATG |
| | | | GTTTTAAAGCTAAACGTAATTTCCACTTGGGACTAGATCAC |
| | | | AACTGAAGATAACAAGAGATTTAAGTTTTAAGGGCATTTAA |
| | | | TCAGGAGGAAAGGTTTGGAAAACTAACTCAGGTGTATTTAT |
| | | | TGTTTAAGCAGAAATAAAGTTTAATTTTTGCTTGAA |
| 5 | SLC22A5 | NM_ | CGCCTTCGCCGGCGCCGCTCTGCCTGCCAGCGGGGCGCGCC |
| | | 001308122.1 | TTGCGGCCCAGGCCCGCAACCTTCCCTGGTCGTGCGCCCTA |
| | | | TGTAAGGCCAGCCGCGGCAGGACCAAGGCGGCGGTGTCAG |
| | | | CTCGCGAGCCTACCCTCCGCGGACGGTCTTGGGTCGCCTGC |
| | | | TGCCTGGCTTGCCTGGTCGGCGGCGGGTGCCCCGCGCGCAC |
| | | | GCGCAAAGCCCGCCGCGTTCCCCGACCCCAGGCCGCGCTCT |
| | | | GTGGGCCTCTGAGGGCGGCATGCGGGACTACGACGAGGTG |
| | | | ACCGCCTTCCTGGGCGAGTGGGGGCCCTTCCAGCGCCTCAT |
| | | | CTTCTTCCTGCTCAGCGCCAGCATCATCCCCAATGGCTTCAC |
| | | | CGGCCTGTCCTCCGTGTTCCTGATAGCGACCCCGGAGCACC |
| | | | GCTGCCGGGTGCCGGACGCCGCGAACCTGAGCAGCGCCTG |
| | | | GCGCAACCACACTGTCCCACTGCGGCTGCGGGACGGCCGCG |
| | | | AGGTGCCCCACAGCTGCCGCCGCTACCGGCTCGCCACCATC |
| | | | GCCAACTTCTCGGCGCTTGGGCTGGAGCCGGGGCGCGACGT |
| | | | GGACCTGGGGCAGCTGGAGCAGGAGAGCTGTCTGGATGGC |
| | | | TGGGAGTTCAGTCAGGACGTCTACCTGTCCACCATTGTGAC |
| | | | CGAGCAAGACAGTGGGGCCTACAATGCTATGAAAAACAGG |
| | | | ATGGGAAAGAAGCCTGCTCTCTGCCTTCCTGCCCAGTGGAA |
| | | | CCTGGTGTGTGAGGACGACTGGAAGGCCCCACTCACAATCT |
| | | | CCTTGTTCTTCGTGGGTGTGCTGTTGGGCTCCTTCATTTCAG |
| | | | GGCAGCTGTCAGACAGGTTTGGCCGGAAGAATGTGCTGTTC |
| | | | GTGACCATGGGCATGCAGACAGGCTTCAGCTTCCTGCAGAT |
| | | | CTTCTCGAAGAATTTTGAGATGTTTGTCGTGCTGTTTGTCCT |
| | | | TGTAGGCATGGGCCAGATCTCCAACTATGTGGCAGCATTTG |
| | | | TCCTGGGGACAGAAATTCTTGGCAAGTCAGTTCGTATAATA |
| | | | TTCTCTACGTTAGGAGTGTGCATATTTTATGCATTTGGCTAC |
| | | | ATGGTGCTGCCACTGTTTGCTTACTTCATCCGAGACTGGCGG |
| | | | ATGCTGCTGGTGGCGCTGACGATGCCGGGGGTGCTATGCGT |
| | | | GGCACTCTGGTGGTTCATCCCTGAGTCCCCCCGATGGCTCAT |
| | | | CTCTCAGGGACGATTTGAAGAGGCAGAGGTGATCATCCGCA |
| | | | AGGCTGCCAAAGCCAATGGGATTGTTGTGCCTTCCACTATC |
| | | | TTTGACCCGAGTGAGTTACAAGACCTAAGTTCCAAGAAGCA |
| | | | GCAGTCCCACAACATTCTGGATCTGCTTCGAACCTGGAATA |
| | | | TCCGGATGGTCACCATCATGTCCATAATGCTGTGGATGACC |
| | | | ATATCAGTGGGCTATTTTGGGCTTTCGCTTGATACTCCTAAC |
| | | | TTGCATGGGGACATCTTTGTGAACTGCTTCCTTTCAGCGATG |
| | | | GTTGAAGTCCCAGCATATGTGTTGGCCTGGCTGCTGCTGCA |
| | | | ATATTTGCCCCGGCGCTATTCCATGGCCACTGCCCTCTTCCT |
| | | | GGGTGGCAGTGTCCTTCTCTTCATGCAGCTGGTACCCCCAG |
| | | | ACTTGTATTATTTGGCTACAGTCCTGGTGATGGTGGGCAAG |
| | | | TTTGGAGTCACGGCTGCCTTTTCCATGGTCTACGTGTACACA |
| | | | GCCGAGCTGTATCCCACAGTGGTGAGAAACATGGGTGTGGG |
| | | | AGTCAGCTCCACAGCATCCCGCCTGGGCAGCATCCTGTCTC |
| | | | CCTACTTCGTTTACCTTGGTGCCTACGACCGCTTCCTGCCCT |
| | | | ACATTCTCATGGGAAGTCTGACCATCCTGACAGCCATCCTC |
| | | | ACCTTGTTTCTCCCAGAGAGCTTCGGTACCCCACTCCCAGAC |
| | | | ACCATTGACCAGATGCTAAGAGTCAAAGGAATGAAACACA |
| | | | GAAAAACTCCAAGTCACACAAGGATGTTAAAAGATGGTCA |
| | | | AGAAAGGCCCACAATCCTTAAAAGCACAGCCTTCTAACATC |
| | | | GCTTCCAGTAAGGGAGAAACTGAAGAGGAAAGACTGTCTT |
| | | | GCCAGAAATGGCCAGCTTGTGCAGACTCCGAGTCCTTCAGT |
| | | | GACAAAAGGCCTTTGCTGTTTGTCCTCTTGACCTGTGTCTGA |
| | | | CTTGCTCCTGGATGGGCACCCACACTCAGAGGCTACATATG |
| | | | GCCCTAGAGCACCACCTTCCTCTAGGGACACTGGGGCTACC |
| | | | TACAGACAACTTCATCTAAGTCCTAACTATTACAATGATGG |
| | | | ACTCAGCACCTCCAAAGCAGTTAATTTTTCACTAGAACCAG |
| | | | TGAGATCTGGAGGAATGTGAGAAGCATATGCTAAATGTACA |
| | | | TTTTAATTTTAGACTACTTGAAAAGGCCCCTAATAAGGCTA |
| | | | GAGGTCTAAGTCCCCCACCCCTTTCCCCACTCCCCTCTAGTG |
| | | | GTGAACTTTAGAGGAAAAGGAAGTAATTGCACAAGGAGTT |
| | | | TGATTCTTACCTTTTCTCAGTTACAGAGGACATTAACTGGAT |
| | | | CATTGCTTCCCCAGGGCAGGAGAGCGCAGAGCTAGGGAAA |
| | | | GTGAAAGGTAATGAAGATGGAGCAGAATGAGCAGATGCAG |
| | | | ATCACCAGCAAAGTGCACTGATGTGTGAGCTCTTAAGACCA |
| | | | CTCAGCATGACGACTGAGTAGACTTGTTTACATCTGATCAA |
| | | | AGCACTGGGCTTGTCCAGGCTCATAATAAATGCTCCATTGA |
| | | | ATCTACTATTCTTGTTTTCCACTGCTGTGGAAACCTCCTTGC |
| | | | TACTATAGCGTCTTATGTATGGTTTAAAGGAAATTTATCAG |
| | | | GTGAGAGAGATGAGCAACGTTGTCTTTTCTCTCAAAGCTGT |
| | | | AATGTGGGTTTTGTTTTATTGTTTATTTGTTTGTTGTTGTATC |
| | | | CTTTTCTCCTTGTTATTTGCCCTTCAGAATGCACTTGGGAAA |
| | | | GGCTGGTTCCTTAGCCTCCTGGTTTGTGTCTTTTTTTTTTTTT |
| | | | TTTTAAAACAGAATCACTCTGGCAATTGTCTGCAGCTGCCA |
| | | | CTGGTGCAAGGCCTTACCAGCCCTAGCCTCTAGCACTTCTCT |
| | | | AAGTGCCAAAAACAGTGTCATTGTGTGTGTTCCTTTCTTGAT |
| | | | ACTTAGTCATGGGAGGATATTACAAAAAAGAAATTTAAATT |
| | | | GTGTTCATAGTCTTTCAGAGTAGCTCACTTTAGTCCTGTAAC |
| | | | TTTATTGGGTGATATTTTGTGTTCAGTGTAATTGTCTTCTCTT |
| | | | TGCTGATTATGTTACCATGGTACTCCTAAAGCATATGCCTCA |
| | | | CCTGGTTAAAAAAGAACAAACATGTTTTTGTGAAAGCTACT |
| | | | GAAGTGCCTTGGGAAATGAGAAAGTTTTAATAAGTAAAATG |
| | | | ATTTTTTAAATAACAAAAAAAAAAAAAAAAAAAA |

TABLE 2
Carnitine Shuttle Proteins

| SEQ | | | |
|---|---|---|---|
| ID | | Accession | |
| NO | Protein | Number | Amino Acid Sequence |
| 6 | Carnitine | P50416 | MAEAHQAVAFQFTVTPDGIDLRLSHEALRQIYLSGLHSW |
| | palmitoyl- | | KKKFIRFKNGIITGVYPASPSSWLIVVVGVMTTMYAKIDPS |
| | transferase 1A | | LGIIAKINRTLETANCMSSQTKNVVSGVLFGTGLWVALIV |
| | (CPT1A) | | TMRYSLKVLLSYHGWMFTEHGKMSRATKIWMGMVKIFS |
| | | | GRKPMLYSFQTSLPRLPVPAVKDTVNRYLQSVRPLMKEE |
| | | | DFKRMTALAQDFAVGLGPRLQWYLKLKSWWATNYVSD |
| | | | WWEEYIYLRGRGPLMVNSNYYAMDLLYILPTHIQAARAG |
| | | | NAIHAILLYRRKLDREEIKPIRLLGSTIPLCSAQWERMFNTS |
| | | | RIPGEETDTIQHMRDSKHIVVYHRGRYFKVWLYHDGRLL |
| | | | KPREMEQQMQRILDNTSEPQPGEARLAALTAGDRVPWAR |
| | | | CRQAYFGRGKNKQSLDAVEKAAFFVTLDETEEGYRSEDP |
| | | | DTSMDSYAKSLLHGRCYDRWFDKSFTFVVFKNGKMGLN |
| | | | AEHSWADAPIVAHLWEYVMSIDSLQLGYAEDGHCKGDIN |
| | | | PNIPYPTRLQWDIPGECQEVIETSLNTANLLANDVDFHSFP |
| | | | FVAFGKGIIKKCRTSPDAFVQLALQLAHYKDMGKFCLTY |
| | | | EASMTRLFREGRTETVRSCTTESCDFVRAMVDPAQTVEQ |
| | | | RLKLFKLASEKHQHMYRLAMTGSGIDRHLFCLYVVSKYL |
| | | | AVESPFLKEVLSEPWRLSTSQTPQQQVELFDLENNPEYVS |
| | | | SGGGFGPVADDGYGVSYILVGENLINFHISSKFSCPETDSH |
| | | | RFGRHLKEAMTDIITLFGLSSNSKK |
| 7 | Carnitine | Q92523 | MAEAHQAVAFQFTVTPDGVDFRLSREALKHVYLSGINSW |
| | palmitoyl- | | KKRLIRIKNGILRGVYPGSPTSWLVVIMATVGSSFCNVDIS |
| | transferase 1B | | LGLVSCIQRCLPQGCGPYQTPQTRALLSMAIFSTGVWVTG |
| | (CPT1B) | | IFFFRQTLKLLLCYHGWMFEMHGKTSNLTRIWAMCIRLLS |
| | | | SRHPMLYSFQTSLPKLPVPRVSATIQRYLESVRPLLDDEEY |
| | | | YRMELLAKEFQDKTAPRLQKYLVLKSWWASNYVSDWW |
| | | | EEYIYLRGRSPLMVNSNYYVMDLVLIKNTDVQAARLGNII |
| | | | HAMIMYRRKLDREEIKPVMALGIVPMCSYQMERMFNTTR |
| | | | IPGKDTDVLQHLSDSRHVAVYHKGRFFKLWLYEGARLLK |
| | | | PQDLEMQFQRILDDPSPPQPGEEKLAALTAGGRVEWAQA |
| | | | RQAFFSSGKNKAALEAIERAAFFVALDEESYSYDPEDEAS |
| | | | LSLYGKALLHGNCYNRWFDKSFTLISFKNGQLGLNAEHA |
| | | | WADAPIIGHLWEFVLGTDSFHLGYTETGHCLGKPNPALAP |
| | | | PTRLQWDIPKQCQAVIESSYQVAKALADDVELYCFQFLPF |
| | | | GKGLIKKCRTSPDAFVQIALQLAHFRDRGKFCLTYEASMT |
| | | | RMFREGRTETVRSCTSESTAFVQAMMEGSHTKADLRDLF |
| | | | QKAAKKHQNMYRLAMTGAGIDRHLFCLYLVSKYLGVSS |
| | | | PFLAEVLSEPWRLSTSQIPQSQIRMFDPEQHPNHLGAGGGF |
| | | | GPVADDGYGVSYMIAGENTIFFHISSKFSSSETNAQRFGNH |
| | | | IRKALLDIADLFQVPKAYS |
| 8 | Carnitine | O43772 | MADQPKPISPLKNLLAGGFGGVCLVFVGHPLDTVKVRLQ |
| | acylcarnitine | | TQPPSLPGQPPMYSGTFDCFRKTLFREGITGLYRGMAAPII |
| | translocase | | GVTPMFAVCFFGFGLGKKLQQKHPEDVLSYPQLFAAGML |
| | (CACT) | | SGVFTTGIMTPGERIKCLLQIQASSGESKYTGTLDCAKKLY |
| | | | QEFGIRGIYKGTVLTLMRDVPASGMYFMTYEWLKNIFTPE |
| | | | GKRVSELSAPRILVAGGIAGIFNWAVAIPPDVLKSRFQTAP |
| | | | PGKYPNGFRDVLRELIRDEGVTSLYKGFNAVMIRAFPANA |
| | | | ACFLGFEVAMKFLNWATPN |
| 9 | Carnitine | P23786 | MVPRLLLRAWPRGPAVGPGAPSRPLSAGSGPGQYLQRSIV |
| | palmitoyl- | | PTMHYQDSLPRLPIPKLEDTIRRYLSAQKPLLNDGQFRKTE |
| | transferase 2 | | QFCKSFENGIGKELHEQLVALDKQNKHTSYISGPWFDMY |
| | (CPT2) | | LSARDSVVLNFNPFMAFNPDPKSEYNDQLTRATNMTVSAI |
| | | | RFLKTLRAGLLEPEVFHLNPAKSDTITFKRLIRFVPSSLSW |
| | | | YGAYLVNAYPLDMSQYFRLFNSTRLPKPSRDELFTDDKA |
| | | | RHLLVLRKGNFYIFDVLDQDGNIVSPSEIQAHLKYILSDSS |
| | | | PAPEFPLAYLTSENRDIWAELRQKLMSSGNEESLRKVDSA |
| | | | VFCLCLDDFPIKDLVHLSHNMLHGDGTNRWFDKSFNLIIA |
| | | | KDGSTAVHFEHSWGDGVAVLRFFNEVFKDSTQTPAVTPQ |
| | | | SQPATTDSTVTVQKLNFELTDALKTGITAAKEKFDATMKT |
| | | | LTIDCVQFQRGGKEFLKKQKLSPDAVAQLAFQMAFLRQY |
| | | | GQTVATYESCSTAAFKHGRTETIRPASVYTKRCSEAFVRE |
| | | | PSRHSAGELQQMMVECSKYHGQLTKEAAMGQGFDRHLF |
| | | | ALRHLAAAKGIILPELYLDPAYGQINHNVLSTSTLSSPAVN |
| | | | LGGFAPVVSDGFGVGYAVHDNWIGCNVSSYPGRNAREFL |
| | | | QCVEKALEDMFDALEGKSIKS |
| 10 | Organic | O76082 | MRDYDEVTAFLGEWGPFQRLIFFLLSASIIPNGFTGLSVFLI |
| | cation/carnitine | | ATPEHRCRVPDAANLSSAWRNHTVPLRLRDGREVPHSCR |
| | transporter | | RYRLATIANFSALGLEPGRDVDLGQLEQESCLDGWEFSQD |
| | (OCTN2) | | VYLSTIVTEWNLVCEDDWKAPLTISLFFVGVLLGSFISGQL |
| | | | SDRFGRKNVLFVTMGMQTGFSFLQIFSKNFEMFVVLFVLV |
| | | | GMGQISNYVAAFVLGTEILGKSVRIIFSTLGVCIFYAFGYM |
| | | | VLPLFAYFIRDWRMLLVALTMPGVLCVALWWFIPESPRW |
| | | | LISQGRFEEAEVIIRKAAKANGIVVPSTIFDPSELQDLSSKK |
| | | | QQSHNILDLLRTWNIRMVTIMSIMLWMTISVGYFGLSLDT |
| | | | PNLHGDIFVNCFLSAMVEVPAYVLAWLLLQYLPRRYSMA |
| | | | TALFLGGSVLLFMQLVPPDLYYLATVLVMVGKFGVTAAF |
| | | | SMVYVYTAELYPTVVRNMGVGVSSTASRLGSILSPYFVY |
| | | | LGAYDRFLPYILMGSLTILTAILTLFLPESFGTPLPDTIDQM |
| | | | LRVKGMKHRKTPSHTRMLKDGQERPTILKSTAF |

TABLE 3
Fatty Acid Oxidation Cycle Genes

| SEQ | | NCBI | |
|---|---|---|---|
| ID | | Reference | |
| NO | Gene | Number | Nucleotide Sequence |
| 11 | ACADVL | NM_000018.4 | AGAGCTGGGTCAGAGCTCGAGCCAGCGGCGCCCGGAG |
| | | | AGATTCGGAGATGCAGGCGGCTCGGATGGCCGCGAGCT |
| | | | TGGGGCGGCAGCTGCTGAGGCTCGGGGGCGGAAGCTC |
| | | | GCGGCTCACGGCGCTCCTGGGGCAGCCCCGGCCCGGCC |
| | | | CTGCCCGGCGGCCCTATGCCGGGGGTGCCGCTCAGCTG |
| | | | GCTCTGGACAAGTCAGATTCCCACCCCTCTGACGCTCT |
| | | | GACCAGGAAAAAACCGGCCAAGGCGGAATCTAAGTCC |
| | | | TTTGCTGTGGGAATGTTCAAAGGCCAGCTCACCACAGA |
| | | | TCAGGTGTTCCCATACCCGTCCGTGCTCAACGAAGAGC |
| | | | AGACACAGTTTCTTAAAGAGCTGGTGGAGCCTGTGTCC |
| | | | CGTTTCTTCGAGGAAGTGAACGATCCCGCCAAGAATGA |
| | | | CGCTCTGGAGATGGTGGAGGAGACCACTTGGCAGGGCC |
| | | | TCAAGGAGCTGGGGGCCTTTGGTCTGCAAGTGCCCAGT |
| | | | GAGCTGGGTGGTGTGGGCCTTTGCAACACCCAGTACGC |
| | | | CCGTTTGGTGGAGATCGTGGGCATGCATGACCTTGGCG |
| | | | TGGGCATTACCCTGGGGGCCCATCAGAGCATCGGTTTC |
| | | | AAAGGCATCCTGCTCTTTGGCACAAAGGCCCAGAAAGA |
| | | | AAAATACCTCCCCAAGCTGGCATCTGGGGAGACTGTGG |
| | | | CCGCTTTCTGTCTAACCGAGCCCTCAAGCGGGTCAGAT |
| | | | GCAGCCTCCATCCGAACCTCTGCTGTGCCCAGCCCCTGT |
| | | | GGAAAATACTATACCCTCAATGGAAGCAAGCTTTGGAT |
| | | | CAGTAATGGGGGCCTAGCAGACATCTTCACGGTCTTTG |
| | | | CCAAGACACCAGTTACAGATCCAGCCACAGGAGCCGTG |
| | | | AAGGAGAAGATCACAGCTTTTGTGGTGGAGAGGGGCTT |
| | | | CGGGGGCATTACCCATGGGCCCCCTGAGAAGAAGATG |
| | | | GGCATCAAGGCTTCAAACACAGCAGAGGTGTTCTTTGA |
| | | | TGGAGTACGGGTGCCATCGGAGAACGTGCTGGGTGAG |
| | | | GTTGGGAGTGGCTTCAAGGTTGCCATGCACATCCTCAA |
| | | | CAATGGAAGGTTTGGCATGGCTGCGGCCCTGGCAGGTA |
| | | | CCATGAGAGGCATCATTGCTAAGGCGGTAGATCATGCC |
| | | | ACTAATCGTACCCAGTTTGGGGAGAAAATTCACAACTT |
| | | | TGGGCTGATCCAGGAGAAGCTGGCACGGATGGTTATGC |
| | | | TGCAGTATGTAACTGAGTCCATGGCTTACATGGTGAGT |
| | | | GCTAACATGGACCAGGGAGCCACGGACTTCCAGATAG |
| | | | AGGCCGCCATCAGCAAAATCTTTGGCTCGGAGGCAGCC |
| | | | TGGAAGGTGACAGATGAATGCATCCAAATCATGGGGG |
| | | | GTATGGGCTTCATGAAGGAACCTGGAGTAGAGCGTGTG |
| | | | CTCCGAGATCTTCGCATCTTCCGGATCTTTGAGGGGAC |
| | | | AAATGACATTCTTCGGCTGTTTGTGGCTCTGCAGGGCTG |
| | | | TATGGACAAAGGAAAGGAGCTCTCTGGGCTTGGCAGTG |
| | | | CTCTAAAGAATCCCTTTGGGAATGCTGGCCTCCTGCTA |
| | | | GGAGAGGCAGGCAAACAGCTGAGGCGGCGGGCAGGGC |
| | | | TGGGCAGCGGCCTGAGTCTCAGCGGACTTGTCCACCCG |
| | | | GAGTTGAGTCGGAGTGGCGAGCTGGCAGTACGGGCTCT |
| | | | GGAGCAGTTTGCCACTGTGGTGGAGGCCAAGCTGATAA |
| | | | AACACAAGAAGGGGATTGTCAATGAACAGTTTCTGCTG |
| | | | CAGCGGCTGGCAGACGGGGCCATCGACCTCTATGCCAT |
| | | | GGTGGTGGTTCTCTCGAGGGCCTCAAGATCCCTGAGTG |
| | | | AGGGCCACCCCACGGCCCAGCATGAGAAAATGCTCTGT |
| | | | GACACCTGGTGTATCGAGGCTGCAGCTCGGATCCGAGA |
| | | | GGGCATGGCCGCCCTGCAGTCTGACCCCTGGCAGCAAG |
| | | | AGCTCTACCGCAACTTCAAAAGCATCTCCAAGGCCTTG |
| | | | GTGGAGCGGGGTGGTGTGGTCACCAGCAACCCACTTGG |
| | | | CTTCTGAATACTCCCGGCCAGGGCCTGTCCCAGTTATGT |
| | | | GCCTTCCCTCAAGCCAAAGCCGAAGCCCCTTTCCTTAA |
| | | | GGCCCTGGTTTGTCCCGAAGGGGCCTAGTGTTCCCAGC |
| | | | ACTGTGCCTGCTCTCAAGAGCACTTACTGCCTCGCAAA |
| | | | TAATAAAAATTTCTAGCCAGTCA |
| 12 | ACADM | NM_000016.5 | CTGCACCGCGCCGCAAGTCCCCCCACCGTTCAGCGCAA |
| | | | CCGGGCCCTCCCAGCCCCGCCGCCGTCCCCCTCCCCCG |
| | | | CCCTGGCTCTCTTTCCGCGCTGCGGTCAGCCTCGGCGTC |
| | | | CCACAGAGAGGGCCAGAGGTGGAAACGCAGAAAACCA |
| | | | AACCAGGACTATCAGAGATTGCCCGGAGAGGGGATGC |
| | | | GACCCCTCCCCAGGTCGCAGCGACGGCGCACGCAAGG |
| | | | GTCACGGAGCATGCGTTGGCTACCCGGCGCCGGGGACC |
| | | | GCTGCCACCCCGCCTAGCGCAGCGCCCCGTCCTTCCGC |
| | | | AGCCCAACCGCCTCTTCCCGCCCCGCCCCATCCCGCCC |
| | | | ACGGGCTCCAGTGGGCGGGACCAGAGGAGTCCCGCGTT |
| | | | CGGGGAGTATGTCAAGGCCGTGACCCGTGTATTATTGT |
| | | | CCGAGTGGCCGGAACGGGAGCCAACATGGCAGCGGGG |
| | | | TTCGGGCGATGCTGCAGGGTCCTGAGAAGTATTTCTCG |
| | | | TTTTCATTGGAGATCACAGCATACAAAAGCCAATCGAC |
| | | | AACGTGAACCAGGATTAGGATTTAGTTTTGAGTTCACC |
| | | | GAACAGCAGAAAGAATTTCAAGCTACTGCTCGTAAATT |
| | | | TGCCAGAGAGGAAATCATCCCAGTGGCTGCAGAATATG |
| | | | ATAAAACTGGTGAATATCCAGTCCCCCTAATTAGAAGA |
| | | | GCCTGGGAACTTGGTTTAATGAACACACACATTCCAGA |
| | | | GAACTGTGGAGGTCTTGGACTTGGAACTTTTGATGCTT |
| | | | GTTTAATTAGTGAAGAATTGGCTTATGGATGTACAGGG |
| | | | GTTCAGACTGCTATTGAAGGAAATTCTTTGGGGCAAAT |
| | | | GCCTATTATTATTGCTGGAAATGATCAACAAAAGAAGA |
| | | | AGTATTTGGGGAGAATGACTGAGGAGCCATTGATGTGT |
| | | | GCTTATTGTGTAACAGAACCTGGAGCAGGCTCTGATGT |
| | | | AGCTGGTATAAAGACCAAAGCAGAAAAGAAAGGAGAT |
| | | | GAGTATATTATTAATGGTCAGAAGATGTGGATAACCAA |
| | | | CGGAGGAAAAGCTAATTGGTATTTTTTATTGGCACGTT |
| | | | CTGATCCAGATCCTAAAGCTCCTGCTAATAAAGCCTTT |
| | | | ACTGGATTCATTGTGGAAGCAGATACCCCAGGAATTCA |
| | | | GATTGGGAGAAAGGAATTAAACATGGGCCAGCGATGT |
| | | | TCAGATACTAGAGGAATTGTCTTCGAAGATGTGAAAGT |
| | | | GCCTAAAGAAAATGTTTTAATTGGTGACGGAGCTGGTT |
| | | | TCAAAGTTGCAATGGGAGCTTTTGATAAAACCAGACCT |
| | | | GTAGTAGCTGCTGGTGCTGTTGGATTAGCACAAAGAGC |
| | | | TTTGGATGAAGCTACCAAGTATGCCCTGGAAAGGAAAA |
| | | | CTTTCGGAAAGCTACTTGTAGAGCACCAAGCAATATCA |
| | | | TTTATGCTGGCTGAAATGGCAATGAAAGTTGAACTAGC |
| | | | TAGAATGAGTTACCAGAGAGCAGCTTGGGAGGTTGATT |
| | | | CTGGTCGTCGAAATACCTATTATGCTTCTATTGCAAAGG |
| | | | CATTTGCTGGAGATATTGCAAATCAGTTAGCTACTGAT |
| | | | GCTGTGCAGATACTTGGAGGCAATGGATTTAATACAGA |
| | | | ATATCCTGTAGAAAAACTAATGAGGGATGCCAAAATCT |
| | | | ATCAGATTTATGAAGGTACTTCACAAATTCAAAGACTT |
| | | | ATTGTAGCCCGTGAACACATTGACAAGTACAAAAATTA |
| | | | AAAAAATTACTGTAGAAATATTGAATAACTAGAACACA |
| | | | AGCCACTGTTTCAGCTCCAGAAAAAAGAAAGGGCTTTA |
| | | | ACGTTTTTTCCAGTGAAAACAAATCCTCTTATATTAAAT |
| | | | CTAAGCAACTGCTTATTATAGTAGTTTATACTTTTGCTT |
| | | | AACTCTGTTATGTCTCTTAAGCAGGTTTGGTTTTTATTA |
| | | | AAATGATGTGTTTTCTTTAGTACCACTTTACTTGAATTA |
| | | | CATTAACCTAGAAAACTACATAGGTTATTTTGATCTCTT |
| | | | AAGATTAATGTAGCAGAAATTTCTTGGAATTTTATTTTT |
| | | | GTAATGACAGAAAAGTGGGCTTAGAAAGTATTCAAGAT |
| | | | GTTACAAAATTTACATTTAGAAAATATTGTAGTATTTGA |
| | | | ATACTGTCAACTTGACAGTAACTTTGTAGACTTAATGGT |
| | | | ATTATTAAAGTTCTTTTTATTGCAGTTTGGAAAGCATTT |
| | | | GTGAAACTTTCTGTTTGGCACAGAAACAGTCAAAATTT |
| | | | TGACATTCATATTCTCCTATTTTACAGCTACAAGAACTT |
| | | | TCTTGAAAATCTTATTTAATTCTGAGCCCATATTTCACT |
| | | | TACCTTATTTAAAATAAATCAATAAAGCTTGCCTTAAAT |
| | | | TATTTTTATATGACTGTTGGTCTCTAGGTAGCCTTTGGT |
| | | | CTATTGTACACAATCTCATTTCATATGTTTGCATTTTGG |
| | | | CAAAGAACTTAATAAAATTGTTCAGTGCTTATTATCAT |
| | | | ATCTTTCTGTATTTTTTCCAGGAAATTTCATTACTTCGTG |
| | | | TAATAGTGTATATTTCTTGTATTTACTATGATGAAAAAA |
| | | | GGTCGTTTTAATTTTGAATTGAATAAAGTTACCTGTTCA |
| | | | TTTTTTATTAGATATTTTAAAGACTTCAGAAAATATAAA |
| | | | TATGAAATAATTTAAGAACCCAAA |
| 13 | ACADS | NM_000017.4 | ACTCCGGAACAGCGCGCTCGCAGCGGGAGGTCGCGAA |
| | | | GCCTGGGACTGTGTCTGTCGCCCATGGCCGCCGCGCTG |
| | | | CTCGCCCGGGCCTCGGGCCCTGCCCGCAGAGCTCTCTG |
| | | | TCCTAGGGCCTGGCGGCAGTTACACACCATCTACCAGT |
| | | | CTGTGGAACTGCCCGAGACACACCAGATGTTGCTCCAG |
| | | | ACATGCCGGGACTTTGCCGAGAAGGAGTTGTTTCCCAT |
| | | | TGCAGCCCAGGTGGATAAGGAACATCTCTTCCCAGCGG |
| | | | CTCAGGTGAAGAAGATGGGCGGGCTTGGGCTTCTGGCC |
| | | | ATGGACGTGCCCGAGGAGCTTGGCGGTGCTGGCCTCGA |
| | | | TTACCTGGCCTACGCCATCGCCATGGAGGAGATCAGCC |
| | | | GTGGCTGCGCCTCCACCGGAGTCATCATGAGTGTCAAC |
| | | | AACTCTCTCTACCTGGGGCCCATCTTGAAGTTTGGCTCC |
| | | | AAGGAGCAGAAGCAGGCGTGGGTCACGCCTTTCACCA |
| | | | GTGGTGACAAAATTGGCTGCTTTGCCCTCAGCGAACCA |
| | | | GGGAACGGCAGTGATGCAGGAGCTGCGTCCACCACCG |
| | | | CCCGGGCCGAGGGCGACTCATGGGTTCTGAATGGAACC |
| | | | AAAGCCTGGATCACCAATGCCTGGGAGGCTTCGGCTGC |
| | | | CGTGGTCTTTGCCAGCACGGACAGAGCCCTGCAAAACA |
| | | | AGGGCATCAGTGCCTTCCTGGTCCCCATGCCAACGCCT |
| | | | GGGCTCACGTTGGGGAAGAAAGAAGACAAGCTGGGCA |
| | | | TCCGGGGCTCATCCACGGCCAACCTCATCTTTGAGGAC |
| | | | TGTCGCATCCCCAAGGACAGCATCCTGGGGGAGCCAGG |
| | | | GATGGGCTTCAAGATAGCCATGCAAACCCTGGACATGG |
| | | | GCCGCATCGGCATCGCCTCCCAGGCCCTGGGCATTGCC |
| | | | CAGACCGCCCTCGATTGTGCTGTGAACTACGCTGAGAA |
| | | | TCGCATGGCCTTCGGGGCGCCCCTCACCAAGCTCCAGG |
| | | | TCATCCAGTTCAAGTTGGCAGACATGGCCCTGGCCCTG |
| | | | GAGAGTGCCCGGCTGCTGACCTGGCGCGCTGCCATGCT |
| | | | GAAGGATAACAAGAAGCCTTTCATCAAGGAGGCAGCC |
| | | | ATGGCCAAGCTGGCCGCCTCGGAGGCCGCGACCGCCAT |
| | | | CAGCCACCAGGCCATCCAGATCCTGGGCGGCATGGGCT |
| | | | ACGTGACAGAGATGCCGGCAGAGCGGCACTACCGCGA |
| | | | CGCCCGCATCACTGAGATCTACGAGGGCACCAGCGAAA |
| | | | TCCAGCGGCTGGTGATCGCCGGGCATCTGCTCAGGAGC |
| | | | TACCGGAGCTGAGCCCGCGGCGGACTGCCCCAGGACTG |
| | | | CGGGAAGGCGCGGGAGCCAGGGGCCTCCACCCCAACC |
| | | | CCGGCTCAGAGACTGGGCGGCCCGGCGGGGGCTCCCTG |
| | | | GGGACCCCAGATGGGCTCAGTGCTGCCACCCAGATCAG |
| | | | ATCACATGGGAATGAGGCCCTCCGACCATTGGCAGCTC |
| | | | CGCCTCTGGGCCTTTCCGCCTCCTCACCACTGTGCCTCA |
| | | | AGTTCCTCATCTAAGTGGCCCTGGCCTCCTGGGGGCGG |
| | | | GGTTGTGGGGGGGCTGAGCGACACTCAGGGACACCTCA |
| | | | GTTGTCCTCCCGCGGGCCCTGGTGCCCTGGCATGAAGG |
| | | | CCCAGTGCGACAGGCCCTTGGTGGGGTCTGTCTTTTCCT |
| | | | TGAGGTCAGAGGTCAGGAGCAGGGCTGGGGTCAGGAT |
| | | | GACGAGGCCTGGGGTCCTGGTGTTGGGCAGGTGGTGGG |
| | | | GCTGGGCCATGGAGCTGGCCCAGAGGCCCCTCAGCCCT |
| | | | TTGTAAAGTCTGATGAAGGCAGGGGTGGTGATTCATGC |
| | | | TGTGTGACTGACTGTGGGTAATAAACACACCTGTCCCC |
| | | | CA |
| 14 | HADHA | NM_000182.5 | AGAGGCGCTCTCCACTGCTGTCCTCTTCAGCTCAAGAT |
| | | | GGTGGCCTGCCGGGCGATTGGCATCCTCAGCCGCTTTT |
| | | | CTGCCTTCAGGATCCTCCGCTCCCGAGGTTATATATGCC |
| | | | GCAATTTTACAGGGTCTTCTGCTTTGCTGACCAGAACCC |
| | | | ATATTAACTATGGAGTCAAAGGGGATGTGGCAGTTGTT |
| | | | CGAATTAACTCTCCCAATTCAAAGGTAAATACACTGAG |
| | | | TAAAGAGCTACATTCAGAGTTCTCAGAAGTTATGAATG |
| | | | AAATCTGGGCTAGTGATCAAATCAGAAGTGCCGTCCTT |
| | | | ATCTCATCAAAGCCAGGCTGCTTTATTGCAGGTGCTGA |
| | | | TATCAACATGTTAGCCGCTTGCAAGACCCTTCAAGAAG |
| | | | TAACACAGCTATCACAAGAAGCACAGAGAATAGTTGA |
| | | | GAAACTTGAAAAGTCCACAAAGCCTATTGTGGCTGCCA |
| | | | TCAATGGATCCTGCCTGGGAGGAGGACTTGAGGTTGCC |
| | | | ATTTCATGCCAATACAGAATAGCAACAAAAGACAGAA |
| | | | AAACAGTATTAGGTACCCCTGAAGTTTTGCTGGGGGCC |
| | | | TTACCAGGAGCAGGAGGCACACAAAGGCTGCCCAAAA |
| | | | TGGTGGGTGTGCCTGCTGCTTTGGACATGATGCTGACT |
| | | | GGTAGAAGCATTCGTGCAGACAGGGCAAAGAAAATGG |
| | | | GACTGGTTGACCAACTGGTGGAACCCCTGGGACCAGGA |
| | | | CTAAAACCTCCAGAGGAACGGACAATAGAATACCTAG |
| | | | AAGAAGTTGCAATTACTTTTGCCAAAGGACTAGCTGAT |
| | | | AAGAAGATCTCTCCAAAGAGAGACAAGGGATTGGTGG |
| | | | AAAAATTGACAGCGTATGCCATGACTATTCCATTTGTC |
| | | | AGGCAACAGGTTTACAAAAAAGTGGAAGAAAAAGTGC |
| | | | GAAAGCAGACTAAAGGCCTTTATCCTGCACCTCTGAAA |
| | | | ATAATTGATGTGGTAAAGACTGGAATTGAGCAAGGGA |
| | | | GTGATGCCGGTTATCTCTGTGAATCTCAGAAATTTGGA |
| | | | GAGCTTGTAATGACCAAAGAATCAAAGGCCTTGATGGG |
| | | | ACTCTACCATGGTCAGGTCCTGTGCAAGAAGAATAAAT |
| | | | TTGGAGCTCCACAGAAGGATGTTAAGCATCTGGCTATT |
| | | | CTTGGTGCAGGGCTGATGGGAGCAGGCATCGCCCAAGT |
| | | | CTCCGTGGATAAGGGGCTAAAGACTATACTTAAAGATG |
| | | | CCACCCTCACTGCGCTAGACCGAGGACAGCAACAAGTG |
| | | | TTCAAAGGATTGAATGACAAAGTGAAGAAGAAAGCTC |
| | | | TAACATCATTTGAAAGGGATTCCATCTTCAGCAACTTG |
| | | | ACTGGGCAGCTTGATTACCAAGGTTTTGAAAAGGCCGA |
| | | | CATGGTGATTGAAGCTGTGTTTGAGGACCTTAGTCTTA |
| | | | AGCACAGAGTGCTAAAGGAAGTAGAAGCGGTGATTCC |
| | | | AGATCACTGTATCTTTGCCAGTAACACATCTGCTCTCCC |
| | | | AATCAGTGAAATCGCTGCTGTCAGCAAAAGACCTGAGA |
| | | | AGGTGATTGGCATGCACTACTTCTCTCCCGTGGACAAG |
| | | | ATGCAGCTGCTGGAGATTATCACGACCGAGAAAACTTC |
| | | | CAAAGACACCAGTGCTTCAGCTGTAGCAGTTGGTCTCA |
| | | | AGCAGGGGAAGGTCATCATTGTGGTTAAGGATGGACCT |
| | | | GGCTTCTATACTACCAGGTGTCTTGCGCCCATGATGTCT |
| | | | GAAGTCATCCGAATCCTCCAGGAAGGAGTTGACCCGAA |
| | | | GAAGCTGGATTCCCTGACCACAAGCTTTGGCTTTCCTGT |
| | | | GGGTGCCGCCACACTGGTGGATGAAGTTGGTGTGGATG |
| | | | TAGCGAAACATGTGGCGGAAGATCTGGGCAAAGTCTTT |
| | | | GGGGAGCGGTTTGGAGGTGGAAACCCAGAACTGCTGA |
| | | | CACAGATGGTGTCCAAGGGCTTCCTAGGTCGTAAATCT |
| | | | GGGAAGGGCTTTTACATCTATCAGGAGGGTGTGAAGAG |
| | | | GAAGGATTTGAATTCTGACATGGATAGTATTTTAGCGA |
| | | | GTCTGAAGCTGCCTCCTAAGTCTGAAGTCTCATCAGAC |
| | | | GAAGACATCCAGTTCCGCCTGGTGACAAGATTTGTGAA |
| | | | TGAGGCAGTCATGTGCCTGCAAGAGGGGATCTTGGCCA |
| | | | CACCTGCAGAGGGAGACATCGGAGCCGTCTTTGGGCTT |
| | | | GGCTTCCCGCCTTGTCTGGGAGGGCCTTTCCGCTTTGTG |
| | | | GATCTGTATGGCGCCCAGAAGATAGTGGACCGGCTCAA |
| | | | GAAATATGAAGCTGCCTATGGAAAACAGTTCACCCCAT |
| | | | GCCAGCTGCTAGCTGACCATGCTAACAGCCCTAACAAG |
| | | | AAGTTCTACCAGTGAGCAGGCCTCATGCCTCGCTCAGT |
| | | | CAGTGCACTAACCCCAGCTGCCGGCAGTGCTGGTTCTC |
| | | | CAACAGAGTGGTGTCTAGATTTATCAGAGTAACGAGAA |
| | | | GACAAACTCCGGCACTGGGTTTGCTCCCTGATTAAAGT |
| | | | GCCTTCAGCCAAGACCATCTCTCCCTCCTGGTGAAGTGT |
| | | | GACTTCGAATTAGTTTGCACTTCCTGTTGGAAGGTAGA |
| | | | GCCCACTGCTCATTGTATAAGCCCCGAGGCCTAGAGTG |
| | | | GCAGCCAAGAGCCATCTGAAGCCACCTCTCTGCCTGTT |
| | | | CCTCCCAAGAGGCCAGGGTGGCCAGGGGTGGTGAGGG |
| | | | CAGTTCTGCACCCAGCCAAACACATAACAATAAAAACC |
| | | | AAACTCTGTGTCAGCATCTTTGCCCTTCTGGTTTAAACG |
| | | | CCTCCTTCAAAAAGCAATCTGGAAGAAAGCCCTGTGCT |
| | | | TTGGGGGAGTAAGAATGTGTGTGCAGAATTCTAGGCAG |
| | | | CACCTTAGGGAGGGACTGGGATGAGAGAAAGTGGGAC |
| | | | CTGGTGGGCTCAACCACACACACCTGTCTGTGCAGATG |
| | | | CTTTGCCCAGGCTTCTCACCACGGTGTACCGGGATATTA |
| | | | AACCTCTTTCCCCAGCCTGGA |
| 15 | HADHB | NM_000183.3 | ACTTGGACCTGAACCTTGCTCCGAGAGGGAGTCCTCGC |
| | | | GGACGTCAGCCAAGATTCCAGAATGACTATCTTGACTT |
| | | | ACCCCTTTAAAAATCTTCCCACTGCATCAAAATGGGCC |
| | | | CTCAGATTTTCCATAAGACCTCTGAGCTGTTCCTCCCAG |
| | | | CTACGAGCTGCCCCAGCTGTCCAGACCAAAACGAAGAA |
| | | | GACGTTAGCCAAACCCAATATAAGGAATGTTGTGGTGG |
| | | | TGGATGGTGTTCGCACTCCATTTTTGCTGTCTGGCACTT |
| | | | CATATAAAGACCTGATGCCACATGATTTGGCTAGAGCA |
| | | | GCGCTTACGGGTTTGTTGCATCGGACCAGTGTCCCTAA |
| | | | GGAAGTAGTTGATTATATCATCTTTGGTACAGTTATTCA |
| | | | GGAAGTGAAAACAAGCAATGTGGCTAGAGAGGCTGCC |
| | | | CTTGGAGCTGGCTTCTCTGACAAGACTCCTGCTCACACT |
| | | | GTCACCATGGCTTGTATCTCTGCCAACCAAGCCATGAC |
| | | | CACAGGTGTTGGCTTGATTGCTTCTGGCCAGTGTGATGT |
| | | | GATCGTGGCAGGTGGTGTTGAGTTGATGTCCGATGTCC |
| | | | CTATTCGTCACTCAAGGAAAATGAGAAAACTGATGCTT |
| | | | GATCTCAATAAGGCCAAATCTATGGGCCAGCGACTGTC |
| | | | TTTAATCTCTAAATTCCGATTTAATTTCCTAGCACCTGA |
| | | | GCTCCCTGCGGTTTCTGAGTTCTCCACCAGTGAGACCAT |
| | | | GGGCCACTCTGCAGACCGACTGGCCGCTGCCTTTGCTG |
| | | | TTTCTCGGCTGGAACAGGATGAATATGCACTGCGCTCT |
| | | | CACAGTCTAGCCAAGAAGGCACAGGATGAAGGACTCC |
| | | | TTTCTGATGTGGTACCCTTCAAAGTACCAGGAAAAGAT |
| | | | ACAGTTACCAAAGATAATGGCATCCGTCCTTCCTCACT |
| | | | GGAGCAGATGGCCAAACTAAAACCTGCATTCATCAAGC |
| | | | CCTACGGCACAGTGACAGCTGCAAATTCTTCTTTCTTGA |
| | | | CTGATGGTGCATCTGCAATGTTAATCATGGCGGAGGAA |
| | | | AAGGCTCTGGCCATGGGTTATAAGCCGAAGGCATATTT |
| | | | GAGGGATTTTATGTATGTGTCTCAGGATCCAAAAGATC |
| | | | AACTATTACTTGGACCAACATATGCTACTCCAAAAGTT |
| | | | CTAGAAAAGGCAGGATTGACCATGAATGATATTGATGC |
| | | | TTTTGAATTTCATGAAGCTTTCTCGGGTCAGATTTTGGC |
| | | | AAATTTTAAAGCCATGGATTCTGATTGGTTTGCAGAAA |
| | | | ACTACATGGGTAGAAAAACCAAGGTTGGATTGCCTCCT |
| | | | TTGGAGAAGTTTAATAACTGGGGTGGATCTCTGTCCCT |
| | | | GGGACACCCATTTGGAGCCACTGGCTGCAGGTTGGTCA |
| | | | TGGCTGCTGCCAACAGATTACGGAAAGAAGGAGGCCA |
| | | | GTATGGCTTAGTGGCTGCGTGTGCAGCTGGAGGGCAGG |
| | | | GCCATGCTATGATAGTGGAAGCTTATCCAAAATAATAG |
| | | | ATCCAGAAGAAGTGACCTGAAGTTTCTGTGCAACACTC |
| | | | ACACTAGGCAATGCCATTTCAATGCATTACTAAATGAC |
| | | | ATTTGTAGTTCCTAGCTCCTCTTAGGAAAACAGTTCTTG |
| | | | TGGCCTTCTATTAAATAGTTTGCACTTAAGCCTTGCCAG |
| | | | TGTTCTGAGCTTTTCAATAATCAGTTTACTGCTCTTTCA |
| | | | GGGATTTCTAAGCCACCAGAATCTCACATGAGATGTGT |
| | | | GGGTGGTTGTTTTTGGTCTCTGTTGTCACTAAAGACTAA |
| | | | ATGAGGGTTTGCAGTTGGGAAAGAGGTCAACTGAGATT |
| | | | TGGAAATCATCTTTGTAATATTTGCAAATTATACTTGTT |
| | | | CTTATCTGTGTCCTAAAGATGTGTTCTCTATAAAATACA |
| | | | AACCAACGTGCCTAATTAATTATGGAAAAATAATTCAG |
| | | | AATCTAAACACCACTGAAAACTTATAAAAAATGTTTAG |
| | | | ATACATAAATATGGTGGTCAGCGTTAATAAAGTGGAGA |
| | | | AATATTGGA |
| 16 | ECHS1 | NM_004092.4 | GGGCGAGGAGTCCAGAGAGCCATGGCCGCCCTGCGTGT |
| | | | CCTGCTGTCCTGCGTCCGCGGCCCGCTGAGGCCCCCGG |
| | | | TTCGCTGTCCCGCCTGGCGTCCCTTCGCCTCGGGTGCTA |
| | | | ACTTTGAGTACATCATCGCAGAAAAAAGAGGGAAGAA |
| | | | TAACACCGTGGGGTTGATCCAACTGAACCGCCCCAAGG |
| | | | CCCTCAATGCACTTTGCGATGGCCTGATTGACGAGCTC |
| | | | AACCAGGCCCTGAAGACCTTCGAGGAGGACCCGGCCGT |
| | | | GGGGGCCATTGTCCTCACCGGCGGGGATAAGGCCTTTG |
| | | | CAGCTGGAGCTGATATCAAGGAAATGCAGAACCTGAGT |
| | | | TTCCAGGACTGTTACTCCAGCAAGTTCTTGAAGCACTG |
| | | | GGACCACCTCACCCAGGTCAAGAAGCCAGTCATCGCTG |
| | | | CTGTCAATGGCTATGCCTTTGGCGGGGGCTGTGAGCTT |
| | | | GCCATGATGTGTGATATCATCTATGCCGGTGAGAAGGC |
| | | | CCAGTTTGCACAGCCGGAGATCTTAATAGGAACCATCC |
| | | | CAGGTGCGGGCGGCACCCAGAGACTCACCCGTGCTGTT |
| | | | GGGAAGTCGCTGGCGATGGAGATGGTCCTCACTGGTGA |
| | | | CCGGATCTCAGCCCAGGACGCCAAGCAAGCAGGTCTTG |
| | | | TCAGCAAGATTTGTCCTGTTGAGACACTGGTGGAAGAA |
| | | | GCCATCCAGTGTGCAGAAAAAATTGCCAGCAATTCTAA |
| | | | AATTGTAGTAGCGATGGCCAAAGAATCAGTGAATGCAG |
| | | | CTTTTGAAATGACATTAACAGAAGGAAGTAAGTTGGAG |
| | | | AAGAAACTCTTTTATTCAACCTTTGCCACTGATGACCGG |
| | | | AAAGAAGGGATGACCGCGTTTGTGGAAAAGAGAAAGG |
| | | | CCAACTTCAAAGACCAGTGAGAACCAGCTGCCCCTGCT |
| | | | TCACACCTCTGCTTGGAGAGGACAAGTGCAGCCTGTCA |
| | | | GTTTTAGAAGCAAGTAAATCATCCTCTTTTCAAGAGCA |
| | | | GTGTCCGTGGTGTGCAGTTCCTCTCCAATTGCTGCGTGG |
| | | | TCGTGGCCCGACCTCTCACGGCATGACAGCCTTCGTCA |
| | | | CCCAGCCTGTGAGGGTCCTGACTGGAGCACCTTCTAAA |
| | | | TCTAAGATTCTGCTGAGGAGCCCCCGCTGGTCCCTCTG |
| | | | GGCATGCTGTGCTCGGACGGAAAGCGGGGCCTGCGGGT |
| | | | CCTTGTGTCCCTGCCGCTGAAGAATGGGGCTGCTCTGA |
| | | | GGGAAACGCTGTCTGCTGCCTTCATACAGATGCTGATT |
| | | | AAAGTGATAGCGATTCAGATTA |
| 17 | HADH | NM_001184705.2 | CGTGTATACCCGCTCAACGCTGGGACGTTACAGCCAGG |
| | | | GCCAATGGGCAGAGCGGGACTCGAGGCCCCGCCCCCG |
| | | | CCTTGTGGCGTCACGGGGACGCCGGGGGCGCGCGGGCT |
| | | | GCAGGGCCGCGTAGGTCCCCGCCCCCAGAGTCTGGCTT |
| | | | TCCGCGGCTGCCCGCCTCGCGCGTCTTCCCTGCCCGGGT |
| | | | CTCCTCGCTGTCGCCGCCGCTGCCACACCATGGCCTTCG |
| | | | TCACCAGGCAGTTCATGCGTTCCGTGTCCTCCTCGTCCA |
| | | | CCGCCTCGGCCTCGGCCAAGAAGATAATCGTCAAGCAC |
| | | | GTGACGGTCATCGGCGGCGGGCTGATGGGCGCCGGCAT |
| | | | TGCCCAGGTTGCTGCAGCAACTGGTCACACAGTAGTGT |
| | | | TGGTAGACCAGACAGAGGACATCCTGGCAAAATCCAA |
| | | | AAAGGGAATTGAGGAAAGCCTTAGGAAAGTGGCAAAG |
| | | | AAGAAGTTTGCAGAAAACCCTAAGGCCGGCGATGAATT |
| | | | TGTGGAGAAGACCCTGAGCACCATAGCGACCAGCACG |
| | | | GATGCAGCCTCCGTTGTCCACAGCACAGACTTGGTGGT |
| | | | GGAAGCCATCGTGGAGAATCTGAAGGTGAAAAACGAG |
| | | | CTCTTCAAAAGGCTGGACAAGTTTGCTGCTGAACATAC |
| | | | AATCTTTGCCAGCAACACTTCCTCCTTGCAGATTACAAG |
| | | | CATAGCTAATGCCACCACCAGACAAGACCGATTCGCTG |
| | | | GCCTCCATTTCTTCAACCCAGTGCCTGTCATGAAACTTG |
| | | | TGGAGGTCATTAAAACACCAATGACCAGCCAGAAGAC |
| | | | ATTTGAATCTTTGGTAGACTTTAGCAAAGCCCTAGGAA |
| | | | AGCATCCTGTTTCTTGCAAGGACACTCCTGGGTTTATTG |
| | | | TGAACCGCCTCCTGGTTCCATACCTCATGGAAGCAATC |
| | | | AGGCTGTATGAACGAGACTTCCAAACGTGTGGTGATTC |
| | | | TAACTCGGGTTTGGGCTTTTCTTTAAAAGGTGACGCATC |
| | | | CAAAGAAGACATTGACACTGCTATGAAATTAGGAGCCG |
| | | | GTTACCCCATGGGCCCATTTGAGCTTCTAGATTATGTCG |
| | | | GACTGGATACTACGAAGTTCATCGTGGATGGGTGGCAT |
| | | | GAAATGGATGCAGAGAACCCATTACATCAGCCCAGCCC |
| | | | ATCCTTAAATAAGCTGGTAGCAGAGAACAAGTTCGGCA |
| | | | AGAAGACTGGAGAAGGATTTTACAAATACAAGTGATGT |
| | | | GCAGCTTCTCCGGCTCTGAGAAGAACACCTGAGAGCGC |
| | | | TTTCCAGCCAGTGCCCCGAGTGCCTGTGGGAATGCTCTT |
| | | | TGGTCAGACATTCCCTCACACAGTACAGTTTAATAAAT |
| | | | GTGCATTTTGATTGTAATCTATCGAAGTGATTATTACAC |
| | | | CAGTTACAGCAGTAATAGATTCTCCATTAAGAAATAAT |
| | | | TCCCTTTTTTAGTCTGTTCATTTCTGTGTATTTTCTAAAC |
| | | | AGCTTTACACCCTTGGTGCCTTGGAGCAAACATGTTTTT |
| | | | TGAACCTTGTCATTTTTGTGAAGAATTGCCTAGATTCCT |
| | | | TCTCTCATCAACGGGAAAGTACTTCCTCTGAGAGTGCG |
| | | | AGTGCACCATGCTCACTGTTGCTGCGTGGGAGAGTCAC |
| | | | AAGCCACTGGCAAGCAAGTGGTATAGTCTGTGAAGCAC |
| | | | TGCAGCGAGCAGCACCTGGATCTTGCCTTTATAAGAAC |
| | | | ATTTTACTACCTGCAGCTTTGAGTCTTGCCCTACATTTT |
| | | | GGGCATGACATAAGATGTGTCTTTATTCAGCTCGTCGT |
| | | | GAAGATGCTGCTGCTGAATGGGTCAGCATATCTCTGTT |
| | | | TGCATGGTTTGCAGGAGGTCGGTTTTCATGGTCATTCAG |
| | | | TTCCACAGATCTGAATGATTACTGTCTGTCTGTGTCTTT |
| | | | TTTCCATGAGAAATCACTGTTGCAAATTGCCTATAAATT |
| | | | GACTCTACTAAAATAACAATGTTTCAGTCTGAAAATTT |
| | | | GAATTGAAAAAAATGTATAATATAAAATTGTAATACAC |
| | | | TCAAATGATTATAAAAGTAAAAGTTGGTAATTTAGGCA |
| | | | GAAGCTAAAAA |
| 18 | ACAA2 | NM_006111.3 | AGCGTCCCCCACACCACAGACCCGCGCCGCCGACGACC |
| | | | CAGCAGCCGCCATGGCTCTGCTCCGAGGTGTGTTTGTA |
| | | | GTTGCTGCTAAGCGAACGCCCTTTGGAGCTTACGGAGG |
| | | | CCTTCTGAAAGACTTCACTGCTACTGACTTGTCTGAATT |
| | | | TGCTGCCAAGGCTGCCTTGTCTGCTGGCAAAGTCTCAC |
| | | | CTGAAACAGTTGACAGTGTGATTATGGGCAATGTCCTG |
| | | | CAGAGTTCTTCAGATGCTATATATTTGGCAAGGCATGTT |
| | | | GGTTTGCGTGTGGGAATCCCAAAGGAGACCCCAGCTCT |
| | | | CACGATTAATAGGCTCTGTGGTTCTGGTTTTCAGTCCAT |
| | | | TGTGAATGGATGTCAGGAAATTTGTGTTAAAGAAGCTG |
| | | | AAGTTGTTTTATGTGGAGGAACCGAAAGCATGAGCCAA |
| | | | GCTCCCTACTGTGTCAGAAATGTGCGTTTTGGAACCAA |
| | | | GCTTGGATCAGATATCAAGCTGGAAGATTCTTTATGGG |
| | | | TATCATTAACAGATCAGCATGTCCAGCTCCCCATGGCA |
| | | | ATGACTGCAGAGAATCTTGCTGTAAAACACAAAATAAG |
| | | | CAGAGAAGAATGTGACAAATATGCCCTGCAGTCACAGC |
| | | | AGAGATGGAAAGCTGCTAATGATGCTGGCTACTTTAAT |
| | | | GATGAAATGGCACCAATTGAAGTGAAGACAAAGAAAG |
| | | | GAAAACAGACAATGCAGGTAGACGAGCATGCTCGGCC |
| | | | CCAAACCACCCTGGAACAGTTACAGAAACTTCCTCCAG |
| | | | TATTCAAGAAAGATGGAACTGTTACTGCAGGGAATGCA |
| | | | TCGGGTGTAGCTGATGGTGCTGGAGCTGTTATCATAGC |
| | | | TAGTGAAGATGCTGTTAAGAAACATAACTTCACACCAC |
| | | | TGGCAAGAATTGTGGGCTACTTTGTATCTGGATGTGAT |
| | | | CCCTCTATCATGGGTATTGGTCCTGTCCCTGCTATCAGT |
| | | | GGGGCACTGAAGAAAGCAGGACTGAGTCTTAAGGACA |
| | | | TGGATTTGGTAGAGGTGAATGAAGCTTTTGCTCCCCAG |
| | | | TACTTGGCTGTTGAGAGGAGTTTGGATCTTGACATAAG |
| | | | TAAAACCAATGTGAATGGAGGAGCCATTGCTTTGGGTC |
| | | | ACCCACTGGGAGGATCTGGATCAAGAATTACTGCACAC |
| | | | CTGGTTCACGAATTAAGGCGTCGAGGTGGAAAATATGC |
| | | | CGTTGGATCAGCTTGCATTGGAGGTGGCCAAGGTATTG |
| | | | CTGTCATCATTCAGAGCACAGCCTGAAGAGACCAGTGA |
| | | | GCTCACTGTGACCCATCCTTACTCTACTTGGCCAGGCCA |
| | | | CAGTAAAACAAGTGACCTTCAGAGCAGCTGCCACAACT |
| | | | GGCCATGCCCTGCCATTGAAACAGTGATTAAGTTTGAT |
| | | | CAAGCCATGGTGACACAAAAATGCATTGATCATGAATA |
| | | | GGAGCCCATGCTAGAAGTACATTCTCTCAGATTTGAAC |
| | | | CAGTGAAATATGATGTATTTCTGAGCTAAAACTCAACT |
| | | | ATAGAAGACATTAAAAGAAATCGTATTCTTGCCAAGTA |
| | | | ACCACCACTTCTGCCTTAGATAATATGATTATAAGGAA |
| | | | ATCAAATAAATGTTGCCTTAACTTCAGTTAATATTTTCC |
| | | | TGTCATTTATATTTTTAAAAATTTTAAATTGTGATAAGA |
| | | | TACACATTACATAAACTTTACCATCTTAACCCTTTTTTA |
| | | | GCGTACAATTCACTGGTATTAAGTACATTCACATTTTTA |
| | | | TACAAACATCCCCACTTTTTATCAACAGAACTTTTTCAG |
| | | | TCACCACACATGGAAACAATAACTCCTGATTCTCCCAT |
| | | | CCCCCATCCCCTGACAACCACCAGTGTATTTTGTTTCTA |
| | | | TAAATTTGATGACTCGAGGTACCTCATAAGTGAAATTA |
| | | | TAAATATCTGTCCTTTCGTGACTGGCTTATTTTACTTTA |
| | | | CTTTATATAATGTTCTCAAGATTCATCCACCTTATGGTG |
| | | | TAGCATGTGTCAGAATTTCCTTCTTTTTAAAGGCTGAAT |
| | | | AATATTCTGCTGTGTGTATAAACCTTACTTCCTTCTTCC |
| | | | CAGCTTAAAGGCCATCTTTCATCCTTTATTTTCTCCCTTT |
| | | | AAAATGCCCCCACAACACTTCCATTGCTTTATTTGTCTG |
| | | | TTCTAAGACTGGATATCTAGTAGGGCAAGGCCCTATTC |
| | | | TTGTTAACTTCATCAAAGAGCCACTGGAAATTTTAATTA |
| | | | AGATTAAATTGAATTTATGGGTTATACATTTATTGGGG |
| | | | GGAAATTTTTTTTTTTTTTTTTGAGACAGAGTCTCGCTCT |
| | | | GTCCTCCAGGCTGGAGTGCAGTGGCGCGATCTCAGCTT |
| | | | ACTGCAAGCTCCGCCTCCTGGGTTCATGCCATTCTCCTG |
| | | | CCTCAGCCTCCCCAGTAGCTGGGACTACAGGCGCCTGC |
| | | | CACTACGCCCGGCTAATTTTTTGTATTTTTAGTAGAGAT |
| | | | GGGGTTTCACCGTGTTAGCCAGGATGGTCTCGATCTCCT |
| | | | GACCTCGTGATCCACCCGCCTCGGCCTCCCAAAGAAGT |
| | | | GCTGGGATTACAGGCGTGAGCCACTGCACCCGGCCTTT |
| | | | TTTTTTTTTTTTTGAGATAGCATCTTGCTCTGTCACCCAG |
| | | | GCAGAATTGCAGTGGCACAGTCATGGCTCACTGAATAA |
| | | | TAGATGTTAAATAATACTAGATGTTAAATAATAGTATC |
| | | | ATAAGTACCTACACTGTTTCCTCAACCCTTTGCTTATAT |
| | | | GGTTTCCTTCATTTGATTAAAAAGCTGAAGTGGCACAT |
| | | | ACATCCCCCTTTCTGTCATAGAGAGGCAGATGACAAGC |
| | | | GGCCTACCCACGGTTTGGGATAATGGACTAGTGGCAAC |
| | | | AGGCAAGTCCAGCTTTTATTGTTTGGGATCCTTACTGAG |
| | | | AAGCAGCAGGCTTCCTCTACTGTCATAAAAATATTAAA |
| | | | AAGTAAGAGCCCTGTATAATTTCTCATAATAAAACAAT |
| | | | GTTTTGCAGAACA |
| 19 | ACAT1 | NM_000019.3 | GGCCGCTAGGGGTGCGGGGTTGGGGAGGAGGCCGCTA |
| | | | GTCTACGCCTGTGGAGCCGATACTCAGCCCTCTGCGAC |
| | | | CATGGCTGTGCTGGCGGCACTTCTGCGCAGCGGCGCCC |
| | | | GCAGCCGCAGCCCCCTGCTCCGGAGGCTGGTGCAGGAA |
| | | | ATAAGATATGTGGAACGGAGTTATGTATCAAAACCCAC |
| | | | TTTGAAGGAAGTGGTCATAGTAAGTGCTACAAGAACAC |
| | | | CCATTGGATCTTTTTTAGGCAGCCTTTCCTTGCTGCCAG |
| | | | CCACTAAGCTTGGTTCCATTGCAATTCAGGGAGCCATT |
| | | | GAAAAGGCAGGGATTCCAAAAGAAGAAGTGAAAGAAG |
| | | | CATACATGGGTAATGTTCTACAAGGAGGTGAAGGACAA |
| | | | GCTCCTACAAGGCAGGCAGTATTGGGTGCAGGCTTACC |
| | | | TATTTCTACTCCATGTACCACCATAAACAAAGTTTGTGC |
| | | | TTCAGGAATGAAAGCCATCATGATGGCCTCTCAAAGTC |
| | | | TTATGTGTGGACATCAGGATGTGATGGTGGCAGGTGGG |
| | | | ATGGAGAGCATGTCCAATGTTCCATATGTAATGAACAG |
| | | | AGGATCAACACCATATGGTGGGGTAAAGCTTGAAGATT |
| | | | TGATTGTAAAAGACGGGCTAACTGATGTCTACAATAAA |
| | | | ATTCATATGGGCAGCTGTGCTGAGAATACAGCAAAGAA |
| | | | GCTGAATATTGCACGAAATGAACAGGACGCTTATGCTA |
| | | | TTAATTCTTATACCAGAAGTAAAGCAGCATGGGAAGCT |
| | | | GGGAAATTTGGAAATGAAGTTATTCCTGTCACAGTTAC |
| | | | AGTAAAAGGTCAACCAGATGTAGTGGTGAAAGAAGAT |
| | | | GAAGAATATAAACGTGTTGATTTTAGCAAAGTTCCAAA |
| | | | GCTGAAGACAGTTTTCCAGAAAGAAAATGGCACAGTA |
| | | | ACAGCTGCCAATGCCAGTACACTGAATGATGGAGCAGC |
| | | | TGCTCTGGTTCTCATGACGGCAGATGCAGCGAAGAGGC |
| | | | TCAATGTTACACCACTGGCAAGAATAGTAGCATTTGCT |
| | | | GACGCTGCTGTAGAACCTATTGATTTTCCAATTGCTCCT |
| | | | GTATATGCTGCATCTATGGTTCTTAAAGATGTGGGATTG |
| | | | AAAAAAGAAGATATTGCAATGTGGGAAGTAAATGAAG |
| | | | CCTTTAGTCTGGTTGTACTAGCAAACATTAAAATGTTGG |
| | | | AGATTGATCCCCAAAAAGTGAATATCAATGGAGGAGCT |
| | | | GTTTCTCTGGGACATCCAATTGGGATGTCTGGAGCCAG |
| | | | GATTGTTGGTCATTTGACTCATGCCTTGAAGCAAGGAG |
| | | | AATACGGTCTTGCCAGTATTTGCAATGGAGGAGGAGGT |
| | | | GCTTCTGCCATGCTAATTCAGAAGCTGTAGACAACCTC |
| | | | TGCTATTTAAGGAGACAACCCTATGTGACCAGAAGGCC |
| | | | TGCTGTAATCAGTGTGACTACTGTGGGTCAGCTTATATT |
| | | | CAGATAAGCTGTTTCATTTTTTATTATTTTCTATGTTAAC |
| | | | TTTTAAAAATCAAAATGATGAAATCCCAAAACATTTTG |
| | | | AAATTAAAAATAAATTTCTTCTTCTGCTTTTTTCTTGGT |
| | | | AACCTTGAAAAGTTTGATACATTTTTGCATTCTGAGTCT |
| | | | ATACTTATCGAAATATGGTAGAAATACCAATGTGTAAT |
| | | | ATTAGTGACTTACATAAGTAGCTAGAAGTTTCCATTTGT |
| | | | GAGAACACATTTATATTTTTGAGGATTGTTAAAGGTCA |
| | | | AGTGAATGCTCTTTATAGGTAATTTACATTTAGTAAATT |
| | | | ACGGTAAATTAAATTACTTCTCTTTACAGTAAGAGTTG |
| | | | GCTATTCTGGACAAACTAGCAGTGCTTCATATAATCAC |
| | | | TCAAACCACAGTGTGTGCAGCAGTACTAGAAACAAGAC |
| | | | AGAAGCCCATGTCCTCAGGGTCTAGAGTGGGGGCAATT |
| | | | TCTTATAACCTCAACATTCAGGGTTGGGGGAGGTCAAG |
| | | | CAGAAAACCCTGGAGTTTGGGCTCTGAATTACTATAGC |
| | | | AGCATAGAGAGTGGGAAGGGAGGTAGAAACTGATATG |
| | | | CTGAATGGATATATAAAAAAGGGAACAGATCACCACTT |
| | | | CCAATACACGACAATGCCTGTTCTTAAGCAGGACAGAC |
| | | | TGTAACAGAAGTATCTCGCATTGCATTTTATCTGGGAA |
| | | | AAAAAAAAAAAAAAA |
| 20 | ACADL | NM_001608.4 | GTATTCCCTCGCGACCAGCCTGTGGCGTGGTTGGGGCT |
| | | | CCGGAAGGGCGCGCGCGAGCGCTTTTTTGGGAGGACAC |
| | | | CACAGGTGGACGCCTCAGCTGATCGTCCTCCCTCCCGG |
| | | | GGACCCTGCCCCGAGTCGCCGAGTAGCCGCAGAGTCGC |
| | | | CTCCGTCGCCCCGCCGCCCCTGTGTTTCGGACATGGCCG |
| | | | CACGCCTTCTCCGAGGGTCCCTACGCGTCCTGGGCGGC |
| | | | CACCGTGCGCCGCGCCAGCTGCCCGCCGCGCGATGTTC |
| | | | TCATTCCGGAGGGGAAGAACGTCTAGAAACTCCTTCTG |
| | | | CTAAAAAATTAACAGATATAGGAATTCGAAGAATCTTT |
| | | | TCTCCAGAGCATGACATTTTCCGGAAAAGTGTAAGGAA |
| | | | GTTTTTCCAAGAAGAAGTGATTCCTCATCACTCAGAAT |
| | | | GGGAGAAAGCTGGAGAAGTAAGTAGGGAGGTTTGGGA |
| | | | AAAAGCTGGAAAACAAGGACTGCTTGGTGTCAATATTG |
| | | | CAGAGCATCTTGGTGGAATTGGAGGGGATCTGTACTCC |
| | | | GCAGCTATTGTCTGGGAGGAGCAAGCTTATTCAAATTG |
| | | | TTCAGGCCCAGGTTTTAGTATTCATTCAGGTATTGTCAT |
| | | | GTCCTATATTACAAACCATGGCTCAGAAGAACAGATTA |
| | | | AGCACTTTATTCCCCAGATGACTGCAGGCAAATGTATT |
| | | | GGTGCAATAGCAATGACAGAGCCTGGAGCTGGAAGTG |
| | | | ACTTACAGGGAATAAAAACAAATGCTAAAAAGGATGG |
| | | | AAGTGACTGGATTCTCAATGGAAGCAAGGTGTTCATCA |
| | | | GTAATGGGTCATTAAGTGATGTTGTGATTGTAGTTGCG |
| | | | GTCACAAATCATGAAGCTCCCTCCCCTGCCCATGGTATT |
| | | | AGCCTTTTTCTGGTGGAAAATGGAATGAAAGGATTTAT |
| | | | CAAGGGACGAAAGCTACATAAAATGGGATTAAAAGCC |
| | | | CAGGATACCGCAGAACTATTCTTTGAAGATATACGGTT |
| | | | GCCAGCTAGTGCCCTACTTGGAGAAGAGAATAAAGGCT |
| | | | TCTATTACATCATGAAAGAGCTTCCACAGGAAAGGCTG |
| | | | TTAATTGCTGATGTGGCAATTTCAGCTAGTGAATTCATG |
| | | | TTTGAAGAAACCAGGAACTATGTTAAACAAAGAAAAG |
| | | | CTTTTGGCAAAACAGTTGCTCACCTACAGACAGTGCAA |
| | | | CATAAATTAGCAGAATTAAAAACACATATATGTGTAAC |
| | | | CCGAGCATTTGTGGACAACTGTCTCCAGCTGCATGAAG |
| | | | CGAAACGTTTGGACTCCGCCACTGCTTGCATGGCGAAA |
| | | | TATTGGGCATCTGAGTTACAAAATAGTGTAGCTTACGA |
| | | | CTGTGTACAGCTCCATGGAGGTTGGGGATACATGTGGG |
| | | | AGTACCCAATTGCAAAAGCTTATGTGGATGCCAGAGTT |
| | | | CAGCCAATCTATGGTGGTACAAATGAAATAATGAAGGA |
| | | | GCTGATTGCAAGAGAGATTGTCTTTGACAAGTAGACAT |
| | | | CTGCCCACATCCTGGAGTCCTATTACAGCTAATCTCGTT |
| | | | TTAAATCTGCTCAAGATAAAATGTAACTTGGAAAGCGA |
| | | | GGAAACACTAAACATGTTTTTACCTGCTCTCTCTATAGA |
| | | | GAAGGAAATAAAATATAAATATAAGATTAACACAGTG |
| | | | GAAGGACAAATCTTTGAAGCCAAAATTCTAGTTTTCCA |
| | | | ATATAAGGTTTAACTTACAGTTTTTTATGTAGCCAAAGG |
| | | | TAAACGGTTTTCTGAATCTTGCCTAGGTGTTTCATTTAT |
| | | | CTCTAAAATTCTAAAAAGCATAAATCATTCAAATCTTC |
| | | | AAACCAAGGCAGAAATAATTTTATGTCGCTATAGTATA |
| | | | AAAACATTAATAAGATAGCACATTGACTTTTAAAGGGA |
| | | | AAAGTAAATATAACTTAGCATGTAAACTCATTTCGGCT |
| | | | ACCATTTGCTCCAAATTCCCTAGAACAGTGGTTTTTACC |
| | | | ACTGTACTCCAACCCCGTTTTTAAGCAATGGAACTCTTT |
| | | | CTTCAAACAAAAGCTTATGCAGAACATCTCTGTGAAAC |
| | | | GCTGCTGAGTGAGAACTGCTTTCATTGAAGCTGGAAGC |
| | | | CATCATACCTTACTGCCTTGAAACCCCTAGGACTCAGCT |
| | | | AAGTATTTGCCTAACCCTGACCAGGGAATGCCTTGGTT |
| | | | CTGTCAATTGCTGACATCTGAGAACACAGAATAATCCA |
| | | | TCATTTTTAATTTCAAGATATTGGTACATTTTATAGGTA |
| | | | TCAAAGCAATGGCTTTTCTTTTGCAACAGTTAATGTATT |
| | | | TATTAACTTAATAATTACTTTATGTCTTCTATAAACCAG |
| | | | GCTGTTAATACAATGATGACAAACAAAACTGGCAAGAT |
| | | | CACTAAAAAATAAGTGAATAAACAAATAAGTAGTAAA |
| | | | ATAAGGTAAGAAGTAAATATGTAAAAGAGATAATTTCA |
| | | | AGCATAAGTGCAATGTAAATAATAAAGTAAGCATTTAA |
| | | | AATTCAAAAGTGAGGAAATGACATTTGATTTAAGACTT |
| | | | AAAAGTAATTACAAAAAATAAACCATTAATTTAAAGTA |
| 21 | ACAD9 | NM_014049.5 | ATCAGACGTGTGTGTGTCCCTGCGGCGCTAAGAAGGGG |
| | | | AGACTGAGGCTGAGGCTGGGGAACATCGGGCAGCATG |
| | | | AGCGGCTGCGGGCTCTTCCTGCGCACCACGGCTGCGGC |
| | | | TCGTGCCTGCCGGGGTCTGGTGGTCTCTACCGCGAACC |
| | | | GGCGGCTACTGCGCACCAGCCCGCCTGTACGAGCTTTC |
| | | | GCCAAAGAGCTTTTCCTAGGCAAAATCAAGAAGAAAG |
| | | | AAGTTTTCCCATTTCCAGAAGTTAGCCAAGATGAACTT |
| | | | AATGAAATCAATCAGTTCTTGGGACCCGTGGAAAAATT |
| | | | CTTCACTGAAGAGGTGGACTCCCGAAAAATTGACCAGG |
| | | | AAGGGAAAATCCCAGATGAAACTTTGGAGAAATTGAA |
| | | | GAGCCTAGGGCTTTTTGGGCTGCAAGTCCCAGAAGAAT |
| | | | ATGGTGGCCTGGGCTTCTCCAACACCATGTACTCAAGA |
| | | | CTAGGGGAGATCATCAGCATGGATGGGTCCATCACTGT |
| | | | GACCCTGGCAGCGCACCAGGCTATTGGCCTCAAGGGGA |
| | | | TCATCTTGGCTGGCACTGAGGAGCAGAAAGCCAAATAC |
| | | | TTGCCTAAACTGGCGTCCGGGGAGCACATTGCAGCCTT |
| | | | CTGCCTCACGGAGCCAGCCAGTGGGAGCGATGCAGCCT |
| | | | CAATCCGGAGCAGAGCCACACTAAGTGAAGACAAGAA |
| | | | GCACTACATCCTCAATGGCTCCAAGGTCTGGATTACTA |
| | | | ATGGAGGACTGGCCAATATTTTTACTGTGTTTGCAAAG |
| | | | ACTGAGGTCGTTGATTCTGATGGATCAGTGAAAGACAA |
| | | | AATCACAGCATTCATAGTAGAAAGAGACTTTGGTGGAG |
| | | | TCACTAATGGGAAACCCGAAGATAAATTAGGCATTCGG |
| | | | GGCTCCAACACTTGTGAAGTCCATTTTGAAAACACCAA |
| | | | GATACCTGTGGAAAACATCCTTGGAGAGGTCGGAGATG |
| | | | GGTTTAAGGTGGCCATGAACATCCTCAACAGCGGCCGG |
| | | | TTCAGCATGGGCAGCGTCGTGGCTGGGCTGCTCAAGAG |
| | | | ATTGATTGAAATGACTGCTGAGTACGCCTGCACAAGGA |
| | | | AACAGTTTAACAAGAGGCTCAGTGAATTTGGATTGATT |
| | | | CAGGAGAAATTTGCACTGATGGCTCAGAAGGCTTACGT |
| | | | CATGGAGAGTATGACCTACCTCACAGCAGGGATGCTGG |
| | | | ACCAACCTGGCTTTCCCGACTGCTCCATCGAGGCAGCC |
| | | | ATGGTGAAGGTGTTCAGCTCCGAGGCCGCCTGGCAGTG |
| | | | TGTGAGTGAGGCGCTGCAGATCCTCGGGGGCTTGGGCT |
| | | | ACACAAGGGACTATCCGTACGAGCGCATACTGCGTGAC |
| | | | ACCCGCATCCTCCTCATCTTCGAGGGAACCAATGAGAT |
| | | | TCTCCGGATGTACATCGCCCTGACGGGTCTGCAGCATG |
| | | | CCGGCCGCATCCTGACTACCAGGATCCATGAGCTTAAA |
| | | | CAGGCCAAAGTGAGCACAGTCATGGATACCGTTGGCCG |
| | | | GAGGCTTCGGGACTCCCTGGGCCGAACTGTGGACCTGG |
| | | | GGCTGACAGGCAACCATGGAGTTGTGCACCCCAGTCTT |
| | | | GCGGACAGTGCCAACAAGTTTGAGGAGAACACCTACTG |
| | | | CTTCGGCCGGACCGTGGAGACACTGCTGCTCCGCTTTG |
| | | | GCAAGACCATCATGGAGGAGCAGCTGGTACTGAAGCG |
| | | | GGTGGCCAACATCCTCATCAACCTGTATGGCATGACGG |
| | | | CCGTGCTGTCGCGGGCCAGCCGCTCCATCCGCATTGGG |
| | | | CTCCGCAACCACGACCACGAGGTTCTCTTGGCCAACAC |
| | | | CTTCTGCGTGGAAGCTTACTTGCAGAATCTCTTCAGCCT |
| | | | CTCTCAGCTGGACAAGTATGCTCCAGAAAACCTAGATG |
| | | | AGCAGATTAAGAAAGTGTCCCAGCAGATCCTTGAGAAG |
| | | | CGAGCCTATATCTGTGCCCACCCTCTGGACAGGACATG |
| | | | CTGAGGCAGGGGACAGTGTCCCCTGCTACCGCCCGCCC |
| | | | CTACCCATGGCCCGTTGCTGGATGACTGTTACTCTTTTT |
| | | | TCAGAAGGTGTTGGGATTATCACAGGTTAAGCCTTTTG |
| | | | TTCCCCGTCTGCACCTGAAGGGTTGTCGCCTGGCCTGG |
| | | | GAGAGCCTCTTCCAGGTTTTGACCTGCAGGCAGTGCTC |
| | | | TCTAACAGGACCATCACAGCTTCTGAACTGAGCCGGAG |
| | | | AGAGAGAATGGAATTGCTGACCCCTGGAACTGGCGGGT |
| | | | ATTCTGGTCATTGAGGAGACACCATAGTGGAAACTGGG |
| | | | GCTTATGCTGCTGCCTCCAGGGTGTGAGGTGGGTGGGG |
| | | | ACCTGTGTCAGGTGTGGATAGCCATTTCTGCTCAACCA |
| | | | CACATTCTCTAAGAAACAGCTTGAAAGCTCTGTCTGGG |
| | | | TCATTCATTTAAACTAGAAGCAGAGGCACTTAAAACAT |
| | | | GTACCAGGAACCATTTAACAAAGAATATAAAATGTCAC |
| | | | AATCTGTGTACTGTTA |

TABLE 4
Fatty Acid Oxidation Cycle Proteins

| SEQ | | | |
|---|---|---|---|
| ID | | Accession | |
| NO | Protein | Number | Amino Acid Sequence |
| 22 | Very long | P49748 | MQAARMAASLGRQLLRLGGGSSRLTALLGQPRPGPARRP |
| | chain acyl-CoA | | YAGGAAQLALDKSDSHPSDALTRKKPAKAESKSFAVGMF |
| | dehydrogenase | | KGQLTTDQVFPYPSVLNEEQTQFLKELVEPVSRFFEEVND |
| | (VLCAD) | | PAKNDALEMVEETTWQGLKELGAFGLQVPSELGGVGLC |
| | | | NTQYARLVEIVGMHDLGVGITLGAHQSIGFKGILLFGTKA |
| | | | QKEKYLPKLASGETVAAFCLTEPSSGSDAASIRTSAVPSPC |
| | | | GKYYTLNGSKLWISNGGLADIFTVFAKTPVTDPATGAVK |
| | | | EKITAFVVERGFGGITHGPPEKKMGIKASNTAEVFFDGVR |
| | | | VPSENVLGEVGSGFKVAMHILNNGRFGMAAALAGTMRGI |
| | | | IAKAVDHATNRTQFGEKIHNFGLIQEKLARMVMLQYVTE |
| | | | SMAYMVSANMDQGATDFQIEAAISKIFGSEAAWKVTDEC |
| | | | IQIMGGMGFMKEPGVERVLRDLRIFRIFEGTNDILRLFVAL |
| | | | QGCMDKGKELSGLGSALKNPFGNAGLLLGEAGKQLRRR |
| | | | AGLGSGLSLSGLVHPELSRSGELAVRALEQFATVVEAKLI |
| | | | KHKKGIVNEQFLLQRLADGAIDLYAMVVVLSRASRSLSE |
| | | | GHPTAQHEKMLCDTWCIEAAARIREGMAALQSDPWQQE |
| | | | LYRNFKSISKALVERGGVVTSNPLGF |
| 23 | Medium-chain | P11310 | MAAGFGRCCRVLRSISRFHWRSQHTKANRQREPGLGFSF |
| | acyl-CoA | | EFTEQQKEFQATARKFAREEIIPVAAEYDKTGEYPVPLIRR |
| | dehydrogenase | | AWELGLMNTHIPENCGGLGLGTFDACLISEELAYGCTGV |
| | (MCAD) | | QTAIEGNSLGQMPIIIAGNDQQKKKYLGRMTEEPLMCAYC |
| | | | VTEPGAGSDVAGIKTKAEKKGDEYIINGQKMWITNGGKA |
| | | | NWYFLLARSDPDPKAPANKAFTGFIVEADTPGIQIGRKEL |
| | | | NMGQRCSDTRGIVFEDVKVPKENVLIGDGAGFKVAMGAF |
| | | | DKTRPVVAAGAVGLAQRALDEATKYALERKTFGKLLVE |
| | | | HQAISFMLAEMAMKVELARMSYQRAAWEVDSGRRNTY |
| | | | YASIAKAFAGDIANQLATDAVQILGGNGFNTEYPVEKLM |
| | | | RDAKIYQIYEGTSQIQRLIVAREHIDKYKN |
| 24 | Short-chain | P16219 | MAAALLARASGPARRALCPRAWRQLHTIYQSVELPETHQ |
| | acyl-CoA | | MLLQTCRDFAEKELFPIAAQVDKEHLFPAAQVKKMGGLG |
| | dehydrogenase | | LLAMDVPEELGGAGLDYLAYAIAMEEISRGCASTGVIMS |
| | (SCAD) | | VNNSLYLGPILKFGSKEQKQAWVTPFTSGDKIGCFALSEP |
| | | | GNGSDAGAASTTARAEGDSWVLNGTKAWITNAWEASAA |
| | | | VVFASTDRALQNKGISAFLVPMPTPGLTLGKKEDKLGIRG |
| | | | SSTANLIFEDCRIPKDSILGEPGMGFKIAMQTLDMGRIGIAS |
| | | | QALGIAQTALDCAVNYAENRMAFGAPLTKLQVIQFKLAD |
| | | | MALALESARLLTWRAAMLKDNKKPFIKEAAMAKLAASE |
| | | | AATAISHQAIQILGGMGYVTEMPAERHYRDARITEIYEGT |
| | | | SEIQRLVIAGHLLRSYRS |
| 25 | Mitochondrial | P40939 | MVACRAIGILSRFSAFRILRSRGYICRNFTGSSALLTRTHIN |
| | trifunctional | | YGVKGDVAVVRINSPNSKVNTLSKELHSEFSEVMNEIWA |
| | protein, alpha | | SDQIRSAVLISSKPGCFIAGADINMLAACKTLQEVTQLSQE |
| | subunit | | AQRIVEKLEKSTKPIVAAINGSCLGGGLEVAISCQYRIATK |
| | (MTPα) | | DRKTVLGTPEVLLGALPGAGGTQRLPKMVGVPAALDMM |
| | | | LTGRSIRADRAKKMGLVDQLVEPLGPGLKPPEERTIEYLE |
| | | | EVAITFAKGLADKKISPKRDKGLVEKLTAYAMTIPFVRQQ |
| | | | VYKKVEEKVRKQTKGLYPAPLKIIDVVKTGIEQGSDAGYL |
| | | | CESQKFGELVMTKESKALMGLYHGQVLCKKNKFGAPQK |
| | | | DVKHLAILGAGLMGAGIAQVSVDKGLKTILKDATLTALD |
| | | | RGQQQVFKGLNDKVKKKALTSFERDSIFSNLTGQLDYQG |
| | | | FEKADMVIEAVFEDLSLKHRVLKEVEAVIPDHCIFASNTS |
| | | | ALPISEIAAVSKRPEKVIGMHYFSPVDKMQLLEIITTEKTSK |
| | | | DTSASAVAVGLKQGKVIIVVKDGPGFYTTRCLAPMMSEVI |
| | | | RILQEGVDPKKLDSLTTSFGFPVGAATLVDEVGVDVAKH |
| | | | VAEDLGKVFGERFGGGNPELLTQMVSKGFLGRKSGKGFY |
| | | | IYQEGVKRKDLNSDMDSILASLKLPPKSEVSSDEDIQFRLV |
| | | | TRFVNEAVMCLQEGILATPAEGDIGAVFGLGFPPCLGGPF |
| | | | RFVDLYGAQKIVDRLKKYEAAYGKQFTPCQLLADHANSP |
| | | | NKKFYQ |
| 26 | Mitochondrial | P55084 | MTILTYPFKNLPTASKWALRFSIRPLSCSSQLRAAPAVQTK |
| | trifunctional | | TKKTLAKPNIRNVVVVDGVRTPFLLSGTSYKDLMPHDLA |
| | protein, beta | | RAALTGLLHRTSVPKEVVDYIIFGTVIQEVKTSNVAREAA |
| | subunit | | LGAGFSDKTPAHTVTMACISANQAMTTGVGLIASGQCDV |
| | (MTPβ) | | IVAGGVELMSDVPIRHSRKMRKLMLDLNKAKSMGQRLSL |
| | | | ISKFRFNFLAPELPAVSEFSTSETMGHSADRLAAAFAVSRL |
| | | | EQDEYALRSHSLAKKAQDEGLLSDVVPFKVPGKDTVTKD |
| | | | NGIRPSSLEQMAKLKPAFIKPYGTVTAANSSFLTDGASAM |
| | | | LIMAEEKALAMGYKPKAYLRDFMYVSQDPKDQLLLGPT |
| | | | YATPKVLEKAGLTMNDIDAFEFHEAFSGQILANFKAMDS |
| | | | DWFAENYMGRKTKVGLPPLEKFNNWGGSLSLGHPFGAT |
| | | | GCRLVMAAANRLRKEGGQYGLVAACAAGGQGHAMIVE |
| | | | AYPK |
| 27 | Short-chain | P30084 | MAALRVLLSCVRGPLRPPVRCPAWRPFASGANFEYIIAEK |
| | enoyl-CoA | | RGKNNTVGLIQLNRPKALNALCDGLIDELNQALKTFEEDP |
| | hydratase | | AVGAIVLTGGDKAFAAGADIKEMQNLSFQDCYSSKFLKH |
| | (Crotonase) | | WDHLTQVKKPVIAAVNGYAFGGGCELAMMCDIIYAGEK |
| | | | AQFAQPEILIGTIPGAGGTQRLTRAVGKSLAMEMVLTGDR |
| | | | ISAQDAKQAGLVSKICPVETLVEEAIQCAEKIASNSKIVVA |
| | | | MAKESVNAAFEMTLTEGSKLEKKLFYSTFATDDRKEGMT |
| | | | AFVEKRKANFKDQ |
| 28 | Short-chain | Q16836 | MAFVTRQFMRSVSSSSTASASAKKIIVKHVTVIGGGLMGA |
| | (S)-3- | | GIAQVAAATGHTVVLVDQTEDILAKSKKGIEESLRKVAK |
| | hydroxyacyl- | | KKFAENLKAGDEFVEKTLSTIATSTDAASVVHSTDLVVEA |
| | CoA | | IVENLKVKNELFKRLDKFAAEHTIFASNTSSLQITSIANATT |
| | dehydrogenase | | RQDRFAGLHFFNPVPVMKLVEVIKTPMTSQKTFESLVDFS |
| | (SCHAD) | | KALGKHPVSCKDTPGFIVNRLLVPYLMEAIRLYERGDASK |
| | | | EDIDTAMKLGAGYPMGPFELLDYVGLDTTKFIVDGWHE |
| | | | MDAENPLHQPSPSLNKLVAENKFGKKTGEGFYKYK |
| 29 | Medium-chain | P42765 | MALLRGVFVVAAKRTPFGAYGGLLKDFTATDLSEFAAKA |
| | 3-ketoacyl- | | ALSAGKVSPETVDSVIMGNVLQSSSDAIYLARHVGLRVGI |
| | CoA thiolase | | PKETPALTINRLCGSGFQSIVNGCQEICVKEAEVVLCGGTE |
| | (MCKAT) | | SMSQAPYCVRNVRFGTKLGSDIKLEDSLWVSLTDQHVQL |
| | | | PMAMTAENLAVKHKISREECDKYALQSQQRWKAANDAG |
| | | | YFNDEMAPIEVKTKKGKQTMQVDEHARPQTTLEQLQKLP |
| | | | PVFKKDGTVTAGNASGVADGAGAVIIASEDAVKKHNFTP |
| | | | LARIVGYFVSGCDPSIMGIGPVPAISGALKKAGLSLKDMD |
| | | | LVEVNEAFAPQYLAVERSLDLDISKTNVNGGAIALGHPLG |
| | | | GSGSRITAHLVHELRRRGGKYAVGSACIGGGQGIAVIIQST |
| | | | A |
| 30 | Acetoacetyl- | P24752 | MAVLAALLRSGARSRSPLLRRLVQEIRYVERSYVSKPTLK |
| | CoA thiolase | | EVVIVSATRTPIGSFLGSLSLLPATKLGSIAIQGAIEKAGIPK |
| | (T2) | | EEVKEAYMGNVLQGGEGQAPTRQAVLGAGLPISTPCTTIN |
| | | | KVCASGMKAIMMASQSLMCGHQDVMVAGGMESMSNVP |
| | | | YVMNRGSTPYGGVKLEDLIVKDGLTDVYNKIHMGSCAE |
| | | | NTAKKLNIARNEQDAYAINSYTRSKAAWEAGKFGNEVIP |
| | | | VTVTVKGQPDVVVKEDEEYKRVDFSKVPKLKTVFQKEN |
| | | | GTVTAANASTLNDGAAALVLMTADAAKRLNVTPLARIV |
| | | | AFADAAVEPIDFPIAPVYAASMVLKDVGLKKEDIAMWEV |
| | | | NEAFSLVVLANIKMLEIDPQKVNINGGAVSLGHPIGMSGA |
| | | | RIVGHLTHALKQGEYGLASICNGGGGASAMLIQKL |
| 31 | Long-chain | P28330 | MAARLLRGSLRVLGGHRAPRQLPAARCSHSGGEERLETP |
| | acyl-CoA | | SAKKLTDIGIRRIFSPEHDIFRKSVRKFFQEEVIPHHSEWEK |
| | dehydrogenase | | AGEVSREVWEKAGKQGLLGVNIAEHLGGIGGDLYSAAIV |
| | (LCAD) | | WEEQAYSNCSGPGFSIHSGIVMSYITNHGSEEQIKHFIPQM |
| | | | TAGKCIGAIAMTEPGAGSDLQGIKTNAKKDGSDWILNGS |
| | | | KVFISNGSLSDVVIVVAVTNHEAPSPAHGISLFLVENGMK |
| | | | GFIKGRKLIAKMGLKAQDTAELFFEDIRLPASALLGEENKG |
| | | | FYYIMKELPQERLLIADVAISASEFMFEETRNYVKQRKAF |
| | | | GKTVAHLQTVQHKLAELKTHICVTRAFVDNCLQLHEAKR |
| | | | LDSATACMAKYWASELQNSVAYDCVQLHGGWGYMWE |
| | | | YPIAKAYVDARVQPIYGGTNEIMKELIAREIVFDK |
| 32 | Acyl-CoA | Q9H845 | SGCGLFLRTTAAARACRGLVVSTANRRLLRTSPPVRAFAK |
| | dehydrogenase | | ELFLGKIKKKEVFPFPEVSQDELNEINQFLGPVEKFFTEEV |
| | 9 (ACAD9) | | DSRKIDQEGKIPDETLEKLKSLGLFGLQVPEEYGGLGFSNT |
| | | | MYSRLGEIISMDGSITVTLAAHQAIGLKGIILAGTEEQKAK |
| | | | YLPKLASGEHIAAFCLTEPASGSDAASIRSRATLSEDKKHY |
| | | | ILNGSKVWITNGGLANIFTVFAKTEVVDSDGSVKDKITAFI |
| | | | VERDFGGVTNGKPEDKLGIRGSNTCEVHFENTKIPVENIL |
| | | | GEVGDGFKVAMNILNSGRFSMGSVVAGLLKRLIEMTAEY |
| | | | ACTRKQFNKRLSEFGLIQEKFALMAQKAYVMESMTYLTA |
| | | | GMLDQPGFPDCSIEAAMVKVFSSEAAWQCVSEALQILGG |
| | | | LGYTRDYPYERILRDTRILLIFEGTNEILRMYIALTGLQHA |
| | | | GRILTTRIHELKQAKVSTVMDTVGRRLRDSLGRTVDLGLT |
| | | | GNHGVVHPSLADSANKFEENTYCFGRTVETLLLRFGKTIM |
| | | | EEQLVLKRVANILINLYGMTAVLSRASRSIRIGLRNHDHE |
| | | | VLLANTFCVEAYLQNLFSLSQLDKYAPENLDEQIKKVSQQ |
| | | | ILEKRAYICAHPLDRTC |

TABLE 5
Auxiliary Enzyme Genes

| SEQ | | NCBI | |
|---|---|---|---|
| ID | | Reference | |
| NO | Gene | Number | Nucleotide Sequence |
| 33 | ECI1 | NM_001178029.1 | AGCCCGCGACCTTTATCCCGCGCGTTGCGGTCAAGATGGCG |
| | | | CTGGTGGCTTCTGTGCGAGTCCCGGCGCGCGTTCTGCTCCGC |
| | | | GCGGGGGCCCGGCTCCCGGGCGCGGCCCTCGGGCGGACGG |
| | | | AGCGGGCGGCCGGCGGCGGAGACGGCGCGCGGCGCTTCGG |
| | | | GAGCCAGCGGGTGCTGGTGGAGCCGGACGCGGGCGCAGGG |
| | | | GTCGCTGTGATGAAATTCAAGAACCCCCCAGTGAACAGCCT |
| | | | GAGCCTGGAGTTTCTGACGGAGCTGGTCATCAGCCTGGAGA |
| | | | AGCTGGAGAATGACAAGAGCTTCCGCGGTGTCATTCTGACC |
| | | | TCGGACCGCCCGGGTGTCTTCTCGGCCGGCCTGGACCTGAC |
| | | | GGAGATGTGTGGGAGGAGCCCCGCCCACTACGCTGGGTACT |
| | | | GGAAGGCCGTTCAGGAGCTGTGGCTGCGGTTGTACCAGTCC |
| | | | AACCTGGTGCTGGTCTCCGCCATCAACGGAGCCTGCCCCGC |
| | | | TGGAGGCTGCCTGGTGGCCCTGACCTGTGACTACCGCATCC |
| | | | TGGCGGACAACCCCAGGTTGAAAGACACCCTGGAGAACAC |
| | | | CATCGGGCACCGGGCGGCGGAGCGTGCCCTGCAGCTGGGG |
| | | | CTGCTCTTCCCGCCGGCGGAGGCCCTGCAGGTGGGCATAGT |
| | | | GGACCAGGTGGTCCCGGAGGAGCAGGTGCAGAGCACTGCG |
| | | | CTGTCAGCGATAGCCCAGTGGATGGCCATTCCAGACCATGC |
| | | | TCGACAGCTGACCAAGGCCATGATGCGAAAGGCCACGGCC |
| | | | AGCCGCCTGGTCACGCAGCGCGATGCGGACGTGCAGAACTT |
| | | | CGTCAGCTTCATCTCCAAAGACTCCATCCAGAAGTCCCTGC |
| | | | AGATGTACTTAGAGAGGCTCAAAGAAGAAAAAGGCTAACG |
| | | | ATTGGGCTGCCACAGGCTTACGGCCACACGTGCCCCTGTGG |
| | | | GTCCCAGGGAGGTCTTAAACAAGGTATTTTTCAACTTAAAA |
| | | | GTACTGCCAGCGTTTCATTTTGCAAAAAAAAAAAAAAAAAA |
| 34 | ECI2 | NM_006117.2 | ACCCCCGAGCCCCCGCAGCCCTAGAGCCGCCCAAGGGATGG |
| | | | CGATGGCGTACTTGGCTTGGAGACTGGCGCGGCGTTCGTGT |
| | | | CCGAGGTCACTAGTTTCCCGGTAGTTCAGCTGCACATGAAT |
| | | | AGAACAGCAATGAGAGCCAGTCAGAAGGACTTTGAAAATT |
| | | | CAATGAATCAAGTGAAACTCTTGAAAAAGGATCCAGGAAA |
| | | | CGAAGTGAAGCTAAAACTCTACGCGCTATATAAGCAGGCCA |
| | | | CTGAAGGACCTTGTAACATGCCCAAACCAGGTGTATTTGAC |
| | | | TTGATCAACAAGGCCAAATGGGACGCATGGAATGCCCTTGG |
| | | | CAGCCTGCCCAAGGAAGCTGCCAGGCAGAACTATGTGGATT |
| | | | TGGTGTCCAGTTTGAGTCCTTCATTGGAATCCTCTAGTCAGG |
| | | | TGGAGCCTGGAACAGACAGGAAATCAACTGGGTTTGAAACT |
| | | | CTGGTGGTGACCTCCGAAGATGGCATCACAAAGATCATGTT |
| | | | CAACCGGCCCAAAAAGAAAAATGCCATAAACACTGAGATG |
| | | | TATCATGAAATTATGCGTGCACTTAAAGCTGCCAGCAAGGA |
| | | | TGACTCAATCATCACTGTTTTAACAGGAAATGGTGACTATT |
| | | | ACAGTAGTGGGAATGATCTGACTAACTTCACTGATATTCCC |
| | | | CCTGGTGGAGTAGAGGAGAAAGCTAAAAATAATGCCGTTTT |
| | | | ACTGAGGGAATTTGTGGGCTGTTTTATAGATTTTCCTAAGCC |
| | | | TCTGATTGCAGTGGTCAATGGTCCAGCTGTGGGCATCTCCGT |
| | | | CACCCTCCTTGGGCTATTCGATGCCGTGTATGCATCTGACAG |
| | | | GGCAACATTTCATACACCATTTAGTCACCTAGGCCAAAGTC |
| | | | CGGAAGGATGCTCCTCTTACACTTTTCCGAAGATAATGAGC |
| | | | CCAGCCAAGGCAACAGAGATGCTTATTTTTGGAAAGAAGTT |
| | | | AACAGCGGGAGAGGCATGTGCTCAAGGACTTGTTACTGAAG |
| | | | TTTTCCCTGATAGCACTTTTCAGAAAGAAGTCTGGACCAGG |
| | | | CTGAAGGCATTTGCAAAGCTTCCCCCAAATGCCTTGAGAAT |
| | | | TTCAAAAGAGGTAATCAGGAAAAGAGAGAGAGAAAAACTA |
| | | | CACGCTGTTAATGCTGAAGAATGCAATGTCCTTCAGGGAAG |
| | | | ATGGCTATCAGATGAATGCACAAATGCTGTGGTGAACTTCT |
| | | | TATCCAGAAAATCAAAACTGTGATGACCACTACAGCAGAGT |
| | | | AAAGCATGTCCAAGGAAGGATGTGCTGTTACCTCTGATTTC |
| | | | CAGTACTGGAACTAAATAAGCTTCATTGTGCCTTTTGTAGTG |
| | | | CTAGAATATCAATTACAATGATGATATTTCACTACAGCTCTG |
| | | | ATGAATAAAAAGTTTTGTAAAACAAGCTTAAGAATTCAAAA |
| | | | AAAAAAAAAAAAAA |
| 35 | DECR1 | NM_001359.2 | GTTCTGGAGACTCAACATGAAGCTACCGGCCAGGGTTTTCT |
| | | | TTACTCTGGGGTCCCGGCTGCCCTGTGGCCTCGCTCCTCGGA |
| | | | GGTTTTTCAGTTATGGGACAAAAATATTATATCAAAACACT |
| | | | GAAGCTTTGCAATCTAAATTCTTTTCACCTCTTCAAAAAGCG |
| | | | ATGCTACCACCTAATAGTTTTCAAGGAAAAGTGGCATTCAT |
| | | | TACTGGGGGAGGTACTGGCCTTGGTAAAGGAATGACAACTC |
| | | | TTCTGTCCAGCCTAGGTGCTCAGTGCGTGATAGCCAGCCGG |
| | | | AAGATGGATGTTTTGAAAGCTACCGCAGAACAAATTTCTTC |
| | | | TCAAACTGGAAATAAGGTTCATGCAATTCAGTGTGATGTGA |
| | | | GGGATCCTGATATGGTTCAAAACACTGTGTCAGAACTGATC |
| | | | AAAGTTGCAGGACATCCTAATATTGTGATAAACAATGCAGC |
| | | | AGGGAATTTTATTTCTCCTACTGAAAGACTTTCTCCTAATGC |
| | | | TTGGAAAACCATAACTGACATAGTTCTAAATGGCACAGCCT |
| | | | TCGTGACACTAGAAATTGGAAAACAACTAATTAAAGCACAG |
| | | | AAAGGAGCAGCATTTCTTTCTATTACTACTATCTATGCTGAG |
| | | | ACTGGTTCAGGTTTTGTAGTACCAAGTGCTTCTGCCAAAGC |
| | | | AGGTGTGGAAGCCATGAGCAAGTCTCTTGCAGCTGAATGGG |
| | | | GTAAATATGGAATGCGATTCAATGTGATTCAACCAGGGCCT |
| | | | ATAAAAACCAAAGGTGCCTTTAGCCGTCTGGACCCAACTGG |
| | | | AACATTTGAGAAAGAAATGATTGGCAGAATTCCCTGTGGTC |
| | | | GCCTGGGGACTGTAGAAGAACTCGCAAATCTTGCTGCTTTC |
| | | | CTTTGTAGTGATTATGCTTCTTGGATTAATGGAGCAGTCATT |
| | | | AAATTTGACGGTGGAGAGGAAGTACTTATTTCAGGGGAATT |
| | | | CAACGACCTGAGAAAGGTCACCAAGGAGCAGTGGGACACC |
| | | | ATAGAAGAACTCATCAGGAAGACAAAAGGTTCCTAAGACC |
| | | | ACTTTGGCCTTCATCTTGGTTACAGAAAAGGGAATAGAAAT |
| | | | GAAACAAATTATCTCTCATCTTTTGACTATTTCAAGTCTAAT |
| | | | AAATTCTTAATTAACAAACATTCATTGAATATGTATTATGTG |
| | | | CCAGGCCAGTGATAGCCATTGTATATTCAAAGATAAATAAA |
| | | | ATGAAATATAGTCCTTCAAAACATTAAAAAAAAAAAAAGG |
| | | | AGGCATGGGGAGAGTAGGTAAAGGCTCCTCTTTACCTATTG |
| | | | ATAGAGGTAAAAAGTACTTAGAAGTGCAGAGAGAACAGAT |
| | | | CTTTGTGACTTGGAAAATCAGGAGAAACTCAATGGTGGCGG |
| | | | TAGCATTTGAGTTACATAATATACTATACCTATATTAATAGG |
| | | | GCCTAAAAGAAAGAAATTAGAGGATACACACTAAATATAA |
| | | | TAGACTTTGCCTTTCCAGTATACTTTCTTTTCACTGGACTTGT |
| | | | GAATTATCTTCTTTGGGTAACTCAGTATTAACTCAAACCTTT |
| | | | AATTTTTACTAGGACCTATTTGTAGCCAGGCATTTTATTTAG |
| | | | TACTGAATAAGCTATAGCCGTTGCCCTTTTTAAATTCATTAT |
| | | | CTAGCAAGATAGTCAAACTTATAAATAATTATTTATGATAC |
| | | | ATTGTGATAAGTATTATTCCAGCAGTATTTAAGTGTAGAGG |
| | | | AGGAAGTAATTCATTCTGTCTCCAGAGTTTGGAGAATGTGA |
| | | | TGCCTAAGAGATAGCATGCCATCCCAGCTGTAAAAGAAGAA |
| | | | TAGATTTCTCTGGGTAAAAGAGGTAAAGAAAGCCTATAAAA |
| | | | TATTTTTGTATATCATTTGATTAAATTTCATCTTTGGTTTGAC |
| | | | TAATTTGTCATCCTGAAAATCAAATAATAATGAATCCAAAG |
| | | | TCTCAAGTCTACAGAGCTATACTTTTGAGCCTATATTTTTAA |
| | | | AATGTCCATTTTGCTTTCCCAGGAGTCAGTTACAACATGTTC |
| | | | ACTAGACTGACTATCCCCATTGCCCAAGTTGACACAAGAGG |
| | | | AAACCAGCTTCCATCTTACCTCATCTGAATAAATCTGCCACA |
| | | | AGCCCATGGAAACCCCAATTAACATTGACAGTTAATTGTGT |
| | | | ACATAAATTACATTTATTACATTTAATTGTGTATATATAGGG |
| | | | GATGTTATAGGTTTGGAATAAGTGGCCCAACATTTCCAATT |
| | | | ATACTGACTTTCACTGGGCTTTTTTTTAGGCTGTTGCACTTTT |
| | | | TCTCCACATGCTTGCAATACAATACTCTCAAAATAAAACGC |
| | | | AGACAGGTACCTAGTCTCCATTTTACCTTTAGTACTAATCCT |
| | | | GTGTATTAGTCTGTTCTCATGCTGCTAATAAAGACATAACCC |
| | | | AAACTGGGTAATTTATAAAAGAAAGAGGTTTCATTGACTCA |
| | | | TAGTTCAGCATGGCTAGGGAGGCCTCACAATCATGGCAGAA |
| | | | GGTGAGTGAGGAGCAAAGTCATGTCTTACGTGGTGGCACCC |
| | | | AAGAGAGCTTGTGCAGGGGAACTCCCATTTATAAAACCATC |
| | | | AGATCTCGTGAGACTTATTCACTATCACACTATTGTGTTGAT |
| | | | ATTGTGTTCACACACCAATAATGATGGTTTATCACTCACTCC |
| | | | ATTTCCAAACCCACCTTCCCACCCACCTCTCACCAAACACAC |
| | | | AAAGACACACTCTTTCCCTCCACTGATTCCACCAGTATAGCC |
| | | | ATATTTCTCTTTCTGGTTAAATTTATACTAAATGTTTACATTT |
| | | | ATATAACTTAATAAATATTATTTTTTTCCA |
| 36 | ECH1 | NM_001398.3 | GCAGTAGACGAAGGCGGCGGCGATGGCGGCGGGGATAGTG |
| | | | GCTTCTCGCAGACTCCGCGACCTACTGACCCGGCGACTGAC |
| | | | AGGCTCCAACTACCCGGGACTCAGTATTAGCCTTCGCCTCA |
| | | | CTGGCTCCTCTGCACAAGAGGAGGCTTCCGGAGTAGCCCTC |
| | | | GGTGAAGCCCCAGACCACAGCTATGAGTCCCTTCGTGTGAC |
| | | | GTCTGCGCAGAAACATGTTCTGCATGTCCAGCTCAACCGGC |
| | | | CCAACAAGAGGAATGCCATGAACAAGGTCTTCTGGAGAGA |
| | | | GATGGTAGAGTGCTTCAACAAGATTTCGAGAGACGCTGACT |
| | | | GTCGGGCGGTGGTGATCTCTGGTGCAGGAAAAATGTTCACT |
| | | | GCAGGTATTGACCTGATGGACATGGCTTCGGACATCCTGCA |
| | | | GCCCAAAGGAGATGATGTGGCCCGGATCAGCTGGTACCTCC |
| | | | GTGACATCATCACTCGATACCAGGAGACCTTCAACGTCATC |
| | | | GAGAGGTGCCCCAAGCCCGTGATTGCTGCCGTCCATGGGGG |
| | | | CTGCATTGGCGGAGGTGTGGACCTTGTCACCGCCTGTGACA |
| | | | TCCGGTACTGTGCCCAGGATGCTTTCTTCCAGGTGAAGGAG |
| | | | GTGGACGTGGGTTTGGCTGCCGATGTAGGAACACTGCAGCG |
| | | | CCTGCCCAAGGTCATCGGGAACCAGAGCCTGGTCAACGAGC |
| | | | TGGCCTTCACCGCCCGCAAGATGATGGCTGACGAGGCCCTG |
| | | | GGCAGTGGGCTGGTCAGCCGGGTGTTCCCAGACAAAGAGGT |
| | | | CATGCTGGATGCTGCCTTAGCGCTGGCGGCCGAGATTTCCA |
| | | | GCAAGAGCCCCGTGGCGGTGCAGAGCACCAAGGTCAACCT |
| | | | GCTGTATTCCCGCGACCATTCGGTGGCCGAGAGCCTCAACT |
| | | | ACGTGGCGTCCTGGAACATGAGCATGCTGCAGACCCAAGAC |
| | | | CTCGTGAAGTCGGTCCAGGCCACGACTGAGAACAAGGAACT |
| | | | GAAAACCGTCACCTTCTCCAAGCTCTGAGAGCCCTCGCGTC |
| | | | CCAGGCCCCAGCCAGGGGGCCGGCCTTGTCCCGCCTCATCC |
| | | | ACAGAAAGGGAGGATGGGCGATGACAGTTGTTTCTATGCCT |
| | | | TCTGACCCAGTTTCCCAGTTTATAACTTTATGACAATGAGTT |
| | | | TCTCAAGCCCAAGGCCTTATCTTCACCCCACAAACAATAAA |
| | | | GCAAAGTAAAGAA |

TABLE 6
Auxiliary Enzymes

| SEQ | | | |
|---|---|---|---|
| ID | | Accession | |
| NO | Protein | Number | Amino Acid Sequence |
| 37 | Δ3, Δ2- | P42126 | MALVASVRVPARVLLRAGARLPGAALGRTERAAGGGDGAR |
| | Enoyl-CoA | | RFGSQRVLVEPDAGAGVAVMKFKNPPVNSLSLEFLTELVISLE |
| | isomerase | | KLENDKSFRGVILTSDRPGVFSAGLDLTEMCGRSPAHYAGYW |
| | 1 (DCI) | | KAVQELWLRLYQSNLVLVSAINGACPAGGCLVALTCDYRILA |
| | | | DNPRYCIGLNETQLGIIAPFWLKDTLENTIGHRAAERALQLGL |
| | | | LFPPAEALQVGIVDQVVPEEQVQSTALSAIAQWMAIPDHARQ |
| | | | LTKAMMRKATASRLVTQRDADVQNFVSFISKDSIQKSLQMYL |
| | | | ERLKEEKG |
| 38 | Δ3, Δ2- | O75521 | MAMAYLAWRLARRSCPSSLQVTSFPVVQLHMNRTAMRASQ |
| | Enoyl-CoA | | KDFENSMNQVKLLKKDPGNEVKLKLYALYKQATEGPCNMP |
| | isomerase | | KPGVFDLINKAKWDAWNALGSLPKEAARQNYVDLVSSLSPS |
| | 2 (PECI) | | LESSSQVEPGTDRKSTGFETLVVTSEDGITKIMFNRPKKKNAIN |
| | | | TEMYHEIMRALKAASKDDSIITVLTGNGDYYSSGNDLTNFTDI |
| | | | PPGGVEEKAKNNAVLLREFVGCFIDFPKPLIAVVNGPAVGISV |
| | | | TLLGLFDAVYASDRATFHTPFSHLGQSPEGCSSYTFPKIMSPA |
| | | | KATEMLIFGKKLTAGEACAQGLVTEVFPDSTFQKEVWTRLKA |
| | | | FAKLPPNALRISKEVIRKREREKLHAVNAEECNVLQGRWLSD |
| | | | ECTNAVVNFLSRKSKL |
| 39 | 2,4- | Q16698 | MKLPARVFFTLGSRLPCGLAPRRFFSYGTKILYQNTEALQSKF |
| | Dienoyl- | | FSPLQKAMLPPNSFQGKVAFITGGGTGLGKGMTTLLSSLGAQ |
| | CoA | | CVIASRKMDVLKATAEQISSQTGNKVHAIQCDVRDPDMVQN |
| | reductase | | TVSELIKVAGHPNIVINNAAGNFISPTERLSPNAWKTITDIVLN |
| | (DECR) | | GTAFVTLEIGKQLIKAQKGAAFLSITTIYAETGSGFVVPSASAK |
| | | | AGVEAMSKSLAAEWGKYGMRFNVIQPGPIKTKGAFSRLDPT |
| | | | GTFEKEMIGRIPCGRLGTVEELANLAAFLCSDYASWINGAVIK |
| | | | FDGGEEVLISGEFNDLRKVTKEQWDTIEELIRKTKGS |
| 40 | Δ3,5- | Q13011 | MAAGIVASRRLRDLLTRRLTGSNYPGLSISLRLTGSSAQEEAS |
| | Δ2,4- | | GVALGEAPDHSYESLRVTSAQKHVLHVQLNRPNKRNAMNK |
| | Dienoyl- | | VFWREMVECFNKISRDADCRAVVISGAGKMFTAGIDLMDMA |
| | CoA | | SDILQPKGDDVARISWYLRDIITRYQETFNVIERCPKPVIAAVH |
| | isomerase | | GGCIGGGVDLVTACDIRYCAQDAFFQVKEVDVGLAADVGTL |
| | (ECH1) | | QRLPKVIGNQSLVNELAFTARKMMADEALGSGLVSRVFPDKE |
| | | | VMLDAALALAAEISSKSPVAVQSTKVNLLYSRDHSVAESLNY |
| | | | VASWNMSMLQTQDLVKSVQATTENKELKTVTFSKL |

What is claimed is:

1. A method for treating a fatty acid oxidation disorder (FAOD) in a mammal comprising administering to the mammal with a FAOD a peroxisome proliferator-activated receptor delta (PPARδ) agonist compound.

2. The method of claim 1, wherein:
treating FAOD comprises improving whole-body fatty acid oxidation (FAO) in the mammal, improving the mammal's exercise tolerance, decreasing pain, decreasing fatigue, or a combination thereof.

3. The method of claim 2, wherein:
improving whole-body fatty acid oxidation in the mammal comprises increasing fatty acid oxidation (FAO) in the mammal.

4. The method of claim 2 or claim 3, wherein:
administration of the PPARδ agonist compound to the mammal normalizes FAO capacities in the mammal, up-regulates gene expression of any one of the enzymes or proteins involved in FAO, increases the activity of an enzyme or protein involved in FAO, or a combination thereof.

5. The method of any one of claims 1-4, wherein:
the fatty acid oxidation disorder comprises one or more defects in one or more of the enzymes or proteins involved in the entry of long-chain fatty acids into mitochondria, intramitochondrial β-oxidation defects of long-chain fatty acids affecting membrane bound enzymes, β-oxidation defects of short- and medium-chain fatty acids affecting enzymes of the mitochondrial matrix, defects in the enzymes or proteins involved with electron transfer to the respiratory chain from mitochondrial β-oxidation, or a combination thereof.

6. The method of any one of claims 1-5, wherein:
the fatty acid oxidation disorder (FAOD) comprises carnitine transporter deficiency, carnitine/acylcarnitine translocase deficiency, carnitine palmitoyl transferase deficiency Type 1, carnitine palmitoyl transferase deficiency Type 2, glutaric acidemia Type 2, long-chain 3-hydroxyacyl CoA dehydrogenase deficiency, medium-chain acyl CoA dehydrogenase deficiency, short-chain acyl CoA dehydrogenase deficiency, short-chain 3-hydroxyacyl CoA dehydrogenase deficiency, trifunctional protein deficiency, or very long-chain acyl CoA dehydrogenase deficiency, or a combination thereof.

7. The method of any one of claims 1-5, wherein:
the fatty acid oxidation disorder comprises carnitine palmitoyltransferase II (CPT2) deficiency, very long-chain Acyl-CoA dehydrogenase (VLCAD) deficiency, long-chain 3-hydroxyacyl-CoA dehydrogenase (LCHAD) deficiency, Trifunctional Protein (TFP) Deficiency; or a combination thereof.

8. The method of any one of claims 1-4, wherein:
the mammal has one or more mutations in one or more of the enzymes or proteins of the mitochondrial fatty acid beta-oxidation pathway.

9. The method of claim 8, wherein:
the enzyme or protein of the mitochondrial fatty acid beta-oxidation pathway is short-chain acyl-CoA dehydrogenase (SCAD), medium-chain acyl-CoA dehydrogenase (MCAD), long-chain 3-hydroxyacyl-CoA dehydrogenase (LCHAD), very long-chain acyl-CoA dehydrogenase (VLCAD), mitochondrial trifunctional protein (TFP), carnitine transporter (CT), Carnitine palmitoyltransferase I (CPT I), carnitine-acylcarnitine translocase (CACT), carnitine palmitoyltransferase II (CPT II), isolated long-chain L3-hydroxyl-CoA dehydrogenase, medium-chain L3-hydroxyl-CoA dehydrogenase, short-chain L3-hydroxyl-CoA dehydrogenase, medium-chain 3-ketoacylCoA thiolase, or long-chain 3-ketoacylCoA thiolase (LCKAT).

10. The method of claim 9, wherein the mutation is:
K304E of MCAD;
L540P, V174M, E609K, or combination thereof, of VLCAD;
E510Q of TFP-alpha subunit (HADHA);
R247C of TFP-beta subunit (HADHB);
or combinations thereof.

11. The method of claim 9, wherein the mutation is a nucleotide mutation in the gene encoding VLCAD.

12. The method of claim 11, wherein the mutation is:
842C>A, 848T>C, 865G>A, 869G>A, 881G>A, 897G>T, 898A>G, 950T>C, 956C>A, 1054A>G, 1096C>T, 1097G>A, 1117A>T, 1001T>G, 1066A>G, 1076C>T, 1153C>T, 1213G>C, 1146G>C, 1310T>C, 1322G>A, 1358G>A, 1360G>A, 1372T>C, 1258A>C, 1388G>A, 1405C>T, 1406G>A, 1430G>A, 1349G>A, 1505T>C, 1396G>T, 1613G>C, 1600G>A, 1367G>A, 1375C>T, 1376G>A, 1532G>A, 1619T>C, 1804C>A, 1844G>A, 1825G>A, 1844G>A, 1837C>G, or a combination thereof.

13. The method of any one of claims 1-12, wherein:
the mammal has elevated creatine kinase (CPK) levels, hepatic dysfunction, cardiomyopathy, hypoglycemia, rhabdomyolysis, acidosis, decreased muscle tone (hypotonia), muscle weakness, exercise intolerance, or combinations thereof.

14. The method of any one of claims 1-13, wherein:
the PPARδ agonist compound binds to and activates the cellular PPARδ and does not substantially activate the cellular peroxisome proliferator activated receptors-alpha (PPARα) and -gamma (PPARγ).

15. The method of any one of claims 1-14, wherein:
the PPARδ agonist compound is a phenoxyalkylcarboxylic acid compound; or a pharmaceutically acceptable salt thereof.

16. The method of any one of claim 15, wherein:
the PPARδ agonist compound is a phenoxyethanoic acid compound, phenoxypropanoic acid compound, phenoxybutanoic acid compound, phenoxypentanoic acid compound, phenoxyhexanoic acid compound, phenoxyoctanoic acid compound, phenoxynonanoic acid compound, or phenoxydecanoic acid compound; or a pharmaceutically acceptable salt thereof.

17. The method of any one of claim 15, wherein:
the PPARδ agonist compound is a phenoxyethanoic acid compound or a phenoxyhexanoic acid compound; or a pharmaceutically acceptable salt thereof.

18. The method of claim 15, wherein:
the PPARδ agonist compound is an allyloxyphenoxyethanoic acid acid compound; or a pharmaceutically acceptable salt thereof.

19. The method of any one of claims 1-18, wherein the PPARδ agonist compound is:
(E)-[4-[3-(4-Fluorophenyl)-3-[4-[3-(morpholin-4-yl)propynyl]phenyl]allyloxy]-2-methyl-phenoxy]acetic acid;
(Z)-[2-Methyl-4-[3-(4-methylphenyl)-3-[4-[3-(morpholin-4-yl)propynyl]phenyl]allyloxy]-phenoxy]acetic acid;
(E)-[2-Methyl-4-[3-[4-[3-(pyrazol-1-yl)prop-1-ynyl]phenyl]-3-(4-trifluoromethylphenyl)-allyloxy]phenoxy]acetic acid;
(E)-[2-Methyl-4-[3-[4-[3-(morpholin-4-yl)propynyl]phenyl]-3-(4-trifluoromethylphenyl)allyloxy]-phenoxy]acetic acid;
(E)-[4-[3-(4-Chlorophenyl)-3-[4-[3-(morpholin-4-yl)propynyl]phenyl]allyloxy]-2-methyl-phenoxy]acetic acid;
(E)-[4-[3-(4-Chlorophenyl)-3-[4-[3-(morpholin-4-yl)propynyl]phenyl]allyloxy]-2-methylphenyl]-propionic acid;
{4-[3-Isobutoxy-5-(3-morpholin-4-yl-prop-1-ynyl)-benzylsulfanyl]-2-methyl-phenoxy}-acetic acid;
{4-[3-Isobutoxy-5-(3-morpholin-4-yl-prop-1-ynyl)-phenylsulfanyl]-2-methyl-phenoxy}-acetic acid; or
{4-[3,3-Bis-(4-bromo-phenyl)-allyloxy]-2-methyl-phenoxy}-acetic acid;
or a pharmaceutically acceptable salt thereof.

20. The method of any one of claims 1-13, wherein the PPARδ agonist is:
(E)-[4-[3-(4-Fluorophenyl)-3-[4-[3-(morpholin-4-yl)propynyl]phenyl]allyloxy]-2-methyl-phenoxy]acetic acid;
(Z)-[2-Methyl-4-[3-(4-methylphenyl)-3-[4-[3-(morpholin-4-yl)propynyl]phenyl]allyloxy]-phenoxy]acetic acid;
(E)-[2-Methyl-4-[3-[4-[3-(pyrazol-1-yl)prop-1-ynyl]phenyl]-3-(4-trifluoromethylphenyl)-allyloxy]phenoxy]acetic acid;
(E)-[2-Methyl-4-[3-[4-[3-(morpholin-4-yl)propynyl]phenyl]-3-(4-trifluoromethylphenyl)allyloxy]-phenoxy]acetic acid;
(E)-[4-[3-(4-Chlorophenyl)-3-[4-[3-(morpholin-4-yl)propynyl]phenyl]allyloxy]-2-methyl-phenoxy]acetic acid;
(E)-[4-[3-(4-Chlorophenyl)-3-[4-[3-(morpholin-4-yl)propynyl]phenyl]allyloxy]-2-methylphenyl]-propionic acid;
{4-[3-Isobutoxy-5-(3-morpholin-4-yl-prop-1-ynyl)-benzylsulfanyl]-2-methyl-phenoxy}-acetic acid;
{4-[3-Isobutoxy-5-(3-morpholin-4-yl-prop-1-ynyl)-phenylsulfanyl]-2-methyl-phenoxy}-acetic acid;
{4-[3,3-Bis-(4-bromo-phenyl)-allyloxy]-2-methyl-phenoxy}-acetic acid;
(R)-3-methyl-6-(2-((5-methyl-2-(4-(trifluoromethyl)phenyl)-1H-imidazol-1-yl)methyl)phenoxy)hexanoic acid;
(R)-3-methyl-6-(2-((5-methyl-2-(6-(trifluoromethyl)pyridin-3-yl)-1H-imidazol-1-yl)methyl)phenoxy)hexanoic acid;
2-{4-[({2-[2-Fluoro-4-(trifluoromethyl)phenyl]-4-methyl-1,3-thiazol-5-yl}methyl)sulfanyl]-2-methylphenoxy}-2-methylpropanoic acid (sodelglitazar; GW677954);
2-[2-methyl-4-[[3-methyl-4-[[4-(trifluoromethyl)phenyl]methoxy]phenyl]thio]phenoxy]-acetic acid;
2-[2-methyl-4-[[[4-methyl-2-[4-(trifluoromethyl)phenyl]-5-thiazolyl]methyl]thio]phenoxy]-acetic acid (GW-501516);
[4-[[[2-[3-Fluoro-4-(trifluoromethyl)phenyl]-4-methyl-5-thiazolyl]methyl]thio]-2-methylphenoxy]acetic acid (GW0742 also known as GW610742);
2-[2,6 dimethyl-4-[3-[4-(methylthio)phenyl]-3-oxo-1(E)-propenyl]phenoxyl]-2-methylpropanoic acid (elafibranor; GFT-505);
{2-methyl-4-[5-methyl-2-(4-trifluoromethyl-phenyl)-2H-[1,2,3]triazol-4-ylmethylsulfanyl]-phenoxy}-acetic acid;
[4-({(2R)-2-Ethoxy-3-[4-(trifluoromethyl)phenoxy]propyl}sulfanyl)-2-methylphenoxy]acetic acid (seladelpar; MBX-8025);
(S)-4-[cis-2,6-dimethyl-4-(4-trifluoromethoxy-phenyl)piperazine-1-sulfonyl]-indan-2-carboxylic acid or a tosylate salt thereof (KD-3010);
(2s)-2-{4-butoxy-3-[({[2-Fluoro-4-(Trifluoromethyl)phenyl]carbonyl}amino)methyl]benzyl}butanoic acid (TIPP-204);
[4-[3-(4-Acetyl-3-hydroxy-2-propylphenoxy)propoxy]phenoxy]acetic acid (L-165,0411);
2-(4-{2-[(4-Chlorobenzoyl)amino]ethyl}phenoxy)-2-methylpropanoic acid (bezafibrate);
2-(2-methyl-4-(((2-(4-(trifluoromethyl)phenyl)-2H-1,2,3-triazol-4-yl)methyl)thio)phenoxy)acetic acid; or
(R)-2-(4-((2-ethoxy-3-(4-(trifluoromethyl)phenoxy)propyl)thio)phenoxy)acetic acid;
or a pharmaceutically acceptable salt thereof.

21. The method of any one of claims 1-20, wherein:
the PPARδ agonist compound is (E)-[4-[3-(4-fluorophenyl)-3-[4-[3-(morpholin-4-yl)propynyl]phenyl]allyloxy]-2-methyl-phenoxy]acetic acid, or a pharmaceutically acceptable salt thereof.

22. The method of any one of claims 1-20, wherein:
the PPARδ agonist compound is (E)-[4-[3-(4-fluorophenyl)-3-[4-[3-(morpholin-4-yl)propynyl]phenyl]allyloxy]-2-methyl-phenoxy]acetic acid, or a pharmaceutically acceptable salt thereof, and is administered to the mammal at a dose of about 10 mg to about 500 mg.

23. The method of any one of claims 1-20, wherein:
the PPARδ agonist compound is (E)-[4-[3-(4-fluorophenyl)-3-[4-[3-(morpholin-4-yl)propynyl]phenyl]allyloxy]-2-methyl-phenoxy]acetic acid or a pharmaceutically acceptable salt thereof, and is administered to the mammal at a dose of about 50 mg to about 200 mg.

24. The method of any one of claims 1-20, wherein:
the PPARδ agonist compound is (E)-[4-[3-(4-fluorophenyl)-3-[4-[3-(morpholin-4-yl)propynyl]phenyl]allyloxy]-2-methyl-phenoxy]acetic acid or a pharmaceutically acceptable salt thereof, and is administered to the mammal at a dose of about 75 mg to about 125 mg.

25. The method of any one of claims 1-20, wherein:
the PPARδ agonist compound is (E)-[4-[3-(4-fluorophenyl)-3-[4-[3-(morpholin-4-yl)propynyl]phenyl]allyloxy]-2-methyl-phenoxy]acetic acid or a pharmaceutically acceptable salt thereof, and is administered to the mammal at a dose of about 50 mg.

26. The method of any one of claims 1-20, wherein:
the PPARδ agonist compound is (E)-[4-[3-(4-fluorophenyl)-3-[4-[3-(morpholin-4-yl)propynyl]phenyl]allyloxy]-2-methyl-phenoxy]acetic acid or a pharmaceutically acceptable salt thereof, and is administered to the mammal at a dose of about 100 mg.

27. The method of any one of claims 1-26, wherein:
the PPARδ agonist compound is systemically administered to the mammal.

28. The method of claim 27, wherein:
the PPARδ agonist compound is administered to the mammal in the form of an oral solution, oral suspension, powder, pill, tablet or capsule.

29. The method of any one of claims 1-28, wherein:
the PPARδ agonist compound is administered to the mammal daily.

30. The method of any one of claims 1-28, wherein:
the PPARδ agonist compound is administered to the mammal once daily.

31. The method of any one of claims 1-30, further comprising:
administering at least one additional therapeutic to the mammal.

32. The method of claim 31, wherein:
the at least one additional therapeutic is ubiquinol, ubiquinone, niacin, riboflavin, creatine, L-carnitine, acetyl-L-carnitine, biotin, thiamine, pantothenic acid, pyridoxine, alpha-lipoic acid, n-heptanoic acid, CoQ10, vitamin E, vitamin C, methylcobalamin, folinic acid, resveratrol, N-acetyl-L-cysteine (NAC), zinc, folinic acid/leucovorin calcium, or a combination thereof.

33. The method of claim 31, wherein:
the at least one additional therapeutic is an odd-chain fatty acid, odd-chain fatty ketone, L-carnitine, or combinations thereof.

34. The method of claim 31, wherein:
the at least one additional therapeutic is triheptanoin, n-heptanoic acid, a triglyceride, or a salt or thereof, or combinations thereof.

35. The method of claim 31, wherein:
the at least one additional therapeutic is an antioxidant.

36. The method of claim 31, wherein:
the at least one additional therapeutic is an additional PPAR agonist.

37. The method of claim 36, wherein:
the additional PPAR agonist is a PPARα agonist, PPARγ agonist, or a pan-PPAR agonist.

38. The method of claim 36, wherein:
the additional PPAR agonist is bezafibrate.

39. The method of any one of claims 1-38, wherein the mammal is a human.

40. A method for treating a fatty acid oxidation disorder (FAOD) in a mammal comprising administering to the mammal with a FAOD a peroxisome proliferator-activated receptor delta (PPARδ) agonist compound, wherein the PPARδ agonist compound is (E)-[4-[3-(4-fluorophenyl)-3-[4-[3-(morpholin-4-yl)propynyl]phenyl]allyloxy]-2-methyl-phenoxy]acetic acid, or a pharmaceutically acceptable salt thereof.

41. The method of claim 40, wherein:
treating FAOD comprises improving whole-body fatty acid oxidation (FAO) in the mammal, improving the mammal's exercise tolerance, decreasing pain, decreasing fatigue, or a combination thereof.

42. The method of claim 41, wherein:
administration of the PPARδ agonist compound to the mammal increases FAO capacities in the mammal, normalizes FAO capacities in the mammal, up-regulates gene expression of any one of the enzymes or proteins involved in FAO, increases the activity of an enzyme or protein involved in FAO, or a combination thereof.

43. The method of any one of claims 40-42, wherein:
the fatty acid oxidation disorder comprises one or more defects in one or more of the enzymes or proteins involved in the entry of long-chain fatty acids into mitochondria, intramitochondrial β-oxidation defects of long-chain fatty acids affecting membrane bound enzymes, β-oxidation defects of short- and medium-chain fatty acids affecting enzymes of the mitochondrial matrix, defects in the enzymes or proteins involved with electron transfer to the respiratory chain from mitochondrial β-oxidation, or a combination thereof.

44. The method of any one of claims 40-43, wherein:
the fatty acid oxidation disorder (FAOD) comprises carnitine transporter deficiency, carnitine/acylcarnitine translocase deficiency, carnitine palmitoyl transferase deficiency Type 1, carnitine palmitoyl transferase deficiency Type 2, glutaric acidemia Type 2, long-chain 3-hydroxyacyl CoA dehydrogenase deficiency, medium-chain acyl CoA dehydrogenase deficiency, short-chain acyl CoA dehydrogenase deficiency, short-chain 3-hydroxyacyl CoA dehydrogenase deficiency, trifunctional protein deficiency, or very long-chain acyl CoA dehydrogenase deficiency, or a combination thereof.

45. The method of any one of claims 40-44, wherein:
the fatty acid oxidation disorder comprises carnitine palmitoyltransferase II (CPT2) deficiency, very long-chain Acyl-CoA dehydrogenase (VLCAD) deficiency, long-chain 3-hydroxyacyl-CoA dehydrogenase (LCHAD) deficiency, Trifunctional Protein (TFP) Deficiency; or a combination thereof.

46. The method of any one of claims 40-43, wherein:
the mammal has one or more mutations in one or more of the enzymes or proteins of the mitochondrial fatty acid beta-oxidation pathway.

47. The method of claim 46, wherein:
the one or more enzymes or proteins of the mitochondrial fatty acid beta-oxidation pathway are selected from the group consisting of short-chain acyl-CoA dehydrogenase (SCAD), medium-chain acyl-CoA dehydrogenase (MCAD), long-chain 3-hydroxyacyl-CoA dehydrogenase (LCHAD), very long-chain acyl-CoA dehydrogenase (VLCAD), mitochondrial trifunctional protein (TFP), carnitine transporter (CT), Carnitine palmitoyltransferase I (CPT I), carnitine-acylcarnitine translocase (CACT), carnitine palmitoyltransferase II (CPT II), isolated long-chain L3-hydroxyl-CoA dehydrogenase, medium-chain L3-hydroxyl-CoA dehydrogenase, short-chain L3-hydroxyl-CoA dehydrogenase, medium-chain 3-ketoacylCoA thiolase, and long-chain 3-ketoacylCoA thiolase (LCKAT).

48. The method of any one of claims 40-47, wherein:
the mammal has elevated creatine kinase (CPK) levels, hepatic dysfunction, cardiomyopathy, hypoglycemia, rhabdomyolysis, acidosis, decreased muscle tone (hypotonia), muscle weakness, exercise intolerance, or combinations thereof.

49. The method of any one of claims 40-48, wherein:
the PPARδ agonist compound is (E)-[4-[3-(4-fluorophenyl)-3-[4-[3-(morpholin-4-yl)propynyl]phenyl]allyloxy]-2-methyl-phenoxy]acetic acid, or a pharmaceutically acceptable salt thereof, and is administered to the mammal at a dose of about 10 mg to about 500 mg.

50. The method of any one of claims 40-48, wherein:
the PPARδ agonist compound is (E)-[4-[3-(4-Fluorophenyl)-3-[4-[3-(morpholin-4-yl)propynyl]phenyl]allyloxy]-2-methyl-phenoxy]acetic acid or a pharmaceutically acceptable salt thereof, and is administered to the mammal at a dose of about 50 mg to about 200 mg.

51. The method of any one of claims 40-48, wherein:
the PPARδ agonist compound is (E)-[4-[3-(4-Fluorophenyl)-3-[4-[3-(morpholin-4-yl)propynyl]phenyl]allyloxy]-2-methyl-phenoxy]acetic acid or a pharmaceutically acceptable salt thereof, and is administered to the mammal at a dose of about 75 mg to about 125 mg.

52. The method of any one of claims 40-48, wherein:
the PPARδ agonist compound is (E)-[4-[3-(4-Fluorophenyl)-3-[4-[3-(morpholin-4-yl)propynyl]phenyl]allyloxy]-2-methyl-phenoxy]acetic acid or a pharmaceutically acceptable salt thereof, and is administered to the mammal at a dose of about 50 mg.

53. The method of any one of claims 40-48, wherein:
the PPARδ agonist compound is (E)-[4-[3-(4-Fluorophenyl)-3-[4-[3-(morpholin-4-yl)propynyl]phenyl]allyloxy]-2-methyl-phenoxy]acetic acid or a pharmaceutically acceptable salt thereof, and is administered to the mammal at a dose of about 100 mg.

54. The method of any one of claims 40-53, wherein:
the PPARδ agonist compound is systemically administered to the mammal in the form of an oral solution, oral suspension, powder, pill, tablet or capsule.

55. The method of claim 54, wherein:
the PPARδ agonist compound is administered to the mammal daily.

56. The method of claim 54, wherein:
the PPARδ agonist compound is administered to the mammal once daily.

57. The method of any one of claims 40-56, further comprising:
administering at least one additional therapeutic to the mammal.

58. The method of claim 57, wherein:
the at least one additional therapeutic is ubiquinol, ubiquinone, niacin, riboflavin, creatine, L-carnitine, acetyl-L-carnitine, biotin, thiamine, pantothenic acid, pyridoxine, alpha-lipoic acid, n-heptanoic acid, CoQ10, vitamin E, vitamin C, methylcobalamin, folinic acid, resveratrol, N-acetyl-L-cysteine (NAC), zinc, folinic acid/leucovorin calcium, or a combination thereof.

59. The method of claim 57, wherein:
the at least one additional therapeutic is an odd-chain fatty acid, odd-chain fatty ketone, L-carnitine, or combinations thereof.

60. The method of claim 57, wherein:
the at least one additional therapeutic is triheptanoin, n-heptanoic acid, a triglyceride, or a salt or thereof, or combinations thereof.

61. The method of claim 57, wherein:
the at least one additional therapeutic is an antioxidant.

62. The method of claim 57, wherein:
the at least one additional therapeutic is bezafibrate.

63. The method of any one of claims 40-62, wherein the mammal is a human.

64. A method for measuring whole-body fatty acid oxidation in a human with a fatty acid oxidation disorder (FAOD) comprising: feeding the human with a fatty acid oxidation disorder (FAOD) a meal comprising ¹³C-enriched fatty acids and measuring the amount of exhaled ¹³CO₂ from the human, wherein the human with a fatty acid oxidation disorder (FAOD) is undergoing treatment with a PPARδ agonist compound.

65. A method for measuring changes in whole-body fatty acid oxidation in a human with a fatty acid oxidation disorder (FAOD) comprising the steps of:
1) providing a meal enriched with a ¹³C labeled fatty acid;
2) administering to the human a PPARδ agonist compound, or a pharmaceutically acceptable salt thereof; and
3) collecting breath samples from the human at regular intervals and measuring for the content of ¹³CO₂ in the breath samples.

66. The method of claim 64 or claim 65, wherein:
the PPARδ agonist binds to and activates the cellular PPARδ and does not substantially activate the cellular peroxisome proliferator activated receptors-alpha (PPARα) and -gamma (PPARγ).

67. The method of any one of claims 64-66, wherein:
the PPARδ agonist compound is a phenoxyalkylcarboxylic acid compound; or a pharmaceutically acceptable salt thereof.

68. The method of any one of claims 64-67, wherein the PPARδ agonist compound is:
(E)-[4-[3-(4-fluorophenyl)-3-[4-[3-(morpholin-4-yl)propynyl]phenyl]allyloxy]-2-methyl-phenoxy]acetic acid; or a pharmaceutically acceptable salt thereof.
